(19) United States
(12) Patent Application Publication
(10) Pub. No.: US 2006/0247250 A1
(43) Pub. Date: Nov. 2, 2006

(54) Pyrimidine inhibitors of kinases

(75) Inventors: Jianguo Cao, San Diego, CA (US); Richard M. Soll, San Diego, CA (US); Glenn Noronha, Oceanside, CA (US); Kathy Barrett, San Diego, CA (US); Colleen Gritzen, Vista, CA (US); John D. Hood, San Diego, CA (US); Chi Ching Mak, San Diego, CA (US); Andrew McPherson, San Diego, CA (US); Ved Pathak, San Diego, CA (US); Joel Renick, San Diego, CA (US); Ute Splittgerber, South San Francisco, CA (US); Binqi Zeng, San Diego, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: TargeGen, Inc., San Diego, CA (US)

(21) Appl. No.: 11/377,234

(22) Filed: Mar. 15, 2006

Related U.S. Application Data
(60) Provisional application No. 60/662,947, filed on Mar. 16, 2005.

(51) Int. Cl. A61K 31/506; C07D 403/14; C07D 403/02

(52) U.S. Cl. 514/252.14; 544/329; 544/295; 514/275

(57) ABSTRACT

The invention provides pyrimidine compounds having formula A. The pyrimidine compounds of the invention are capable of inhibiting kinases, such as members of the Src kinase family, and various other specific receptor and non-receptor kinases.

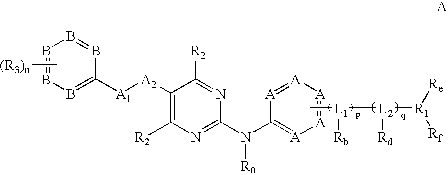

### CROSS REFERENCE TO RELATED APPLICATIONS

This application claims the benefit of priority under 35 U.S.C. § 119(e) to the U.S. patent application Ser. No. 60/662,947 filed Mar. 16, 2005, the entire content of which is incorporated herein by reference.

### FIELD OF THE INVENTION

The present invention relates generally to the use of compounds to treat a variety of disorders, diseases and pathologic conditions and more specifically to the use of pyrimidine compounds to treat various disorders.

### BACKGROUND

Protein kinases are families of enzymes that catalyze the phosphorylation of specific residues in proteins, and may be broadly classified into tyrosine or serine/threonine kinases based on the amino acids phosphorylated. This covalent post-translational modification is a pivotal component of normal cellular communication and maintenance of homeostasis. Tyrosine kinase signaling pathways normally prevent deregulated proliferation or contribute to sensitivity towards apoptotic stimuli. These signaling pathways are often genetically or epigenetically altered in cancer cells to impart a selection advantage to the cancer cells. Understandably therefore, aberrant enhanced signaling emanating from tyrosine kinase endows these enzymes a dominating oncoprotein status, resulting in the malfunctioning of the signaling network. Inappropriate kinase activity arising from mutation, over-expression, or inappropriate regulation, dys-regulation, mis-regulation or de-regulation, as well as over- or under-production of growth factors or cytokines has been implicated in many diseases, including but not limited too cancer, cardiovascular diseases, allergies, asthma and other respiratory diseases, autoimmune diseases, inflammatory diseases, bone diseases, metabolic disorders, and neurological and neurodegenerative disorders such as Alzheimer's disease. Inappropriate kinase activity triggers a variety of biological cellular responses relating to cell growth, cell differentiation, survival, apoptosis, mitogenesis, cell cycle control, and cell mobility implicated in the aforementioned diseases. Current evidence indicates that several distinct families of tyrosine kinases function in each of these responses and that additional complexity results from extensive cross-talk between different receptor pathways. One family of cytoplasmic tyrosine kinases capable of communicating with a large number of different receptors is the Src protein tyrosine kinase family. The c-Src proto-oncogene plays a major role in the development, growth, progression, and metastasis of a wide variety of human cancers. Src over-activation, in the form of elevated kinase activity and protein expression levels, has been demonstrated in several major cancer types, including colon, breast, pancreatic, lung, and brain carcinomas. Src kinase modulates signal transduction through multiple oncogenic pathways, including EGFR, Her2/neu, PDGFR, FGFR, and VEGFR. The prototype member of the Src family of protein tyrosine kinases was first identified as the transforming protein (v-Src) of the oncogenic retrovirus, Rous sarcoma virus. v-Src is a mutant variant of a cellular protein ubiquitously expressed and highly conserved through evolution. The structural and functional interactions between the Src family kinases and cellular receptors, and from Src family kinases on receptor-induced biological activities regulated by these kinases is quite profound.

Thus, it is anticipated that blocking signaling through the inhibition of the kinase activity of Src will be an effective means of modulating aberrant pathways. Gene knockout experiments suggest that inhibition of some members of the Src family might have potential therapeutic benefit.

c-Src is one of three members of the Src family expressed ubiquitously. c-Src is expressed at low levels in most cell types and, in the absence of the appropriate extra-cellular stimuli, maintained in an inactive conformation through phosphorylation of a regulatory tyrosine domain at Tyr530. Activation of c-Src occurs through de-phosphorylation of the Tyr530 site and phosphorylation of a second tyrosine, Tyr419, present in the kinase domain of the enzyme.

There exists a body of evidence of de-regulated or misregulated increased kinase activity of c-Src in several human tumor types, most notably colon and breast tumors. Misregulated c-Src TK activity has also been associated with adhesion and cytoskeletal changes both in tumor cells and otherwise, ultimately resulting in an invasive phenotype that may be motile. c-Src TK activity has been shown to be an important component in the epithelial to mesenchymal transition that occurs in the early stages of invasion of carcinoma cells. c-Src activity is also known to be essential in the turnover of local adhesions, a critical cell-motility component. In in vivo models of metastases, c-Src inhibition markedly reduces the rate of lymph and liver metastases. Clinical data supports the link between misregulated Src activity and the increased invasive potential of tumor cells. In colon tumors, increased c-Src TK activity has been shown to correlate to tumor progression, with the highest activity found in metastatic tissue. Increased Src activity in colon tumors might be an indicator of poor prognosis. In breast and ovarian cancers, enhancement of Src kinase activity has been reported, and in transitional cell carcinoma of the bladder, c-Src activity peaked as superficial tumors became muscle invasive.

Biochemically, cellular stimuli that lead to Src activation result in increased association between Src and the cytoskeleton. As a result, Src mediates the phosphorylation of many intracellular substrates such as EGFR, FAK, PYK2, paxillin, Stat3, and cyclin D. The biological effects of these interactions affect cell motility, adhesion, cell cycle progression, and apoptosis and might have some connection to the disease related effects stated above. Thus, Src plays a role in responses to regional hypoxia, limited nutrients, and internal cellular effects to self-destruct.

Increased c-Src TK activity results in breakdown of the E-cadherin-mediated epithelial cell-cell adhesion, which can be restored by Src inhibition. Intimate connections between increased VEGF activity, Src activity, and cellular barrier function related to vascular leak have been also demonstrated. Inhibition of Src results in decrease in vascular leak when exogenous VEGF is administered in in vivo studies. Examples where excessive vascular permeability leads to particularly deleterious effects include pulmonary edema, cerebral edema, and cardiac edema.

The cascade of events leading to loss of endothelial barrier function is complex and incompletely understood. Data support some role for kinases in this process. For example, VEGF-mediated edema has been shown to involve intracellular signaling by Src family kinases, protein kinase C, and Akt kinase. Rho-associated kinases have been linked to thrombin-mediated vascular leakage, and protein kinase C to TNF-induced leakage. Kinases are believed to mediate the phosphorylation of junctional proteins such as beta-catenin and vascular endothelial VE-cadherin, leading to the dissolution of adherens junctions and the dissociation of cadherin-catenin complexes from their cytoskeletal anchors. Proteins which regulate the intercellular contractile machinery such as myosin light chain kinase (MLCK) and myosin light chain (MLC) are also activated, resulting in cellular contraction, and therefore an opening of intercellular junctions.

A general approach to the inhibition of vascular leakage can be to interfere with any of the underlying mechanistic pathways, whether by inhibition of kinase signaling or the intercellular contractile apparatus or other cellular processes. This can then lead to potential treatments for edema and its associated pathologies. For example, inhibiting edema formation should be beneficial to overall patient outcome in situations such as inflammation, allergic diseases, cancer, cerebral stroke, myocardial infarction, pulmonary and cardiac insufficiency, renal failure, and retinopathies, to name a few. Furthermore, as edema is a general consequence of tissue hypoxia, it can also be concluded that inhibition of vascular leakage represents a potential approach to the treatment of tissue hypoxia. For example, interruption of blood flow by pathologic conditions (such as thrombus formation) or medical intervention (such as cardioplegia, organ transplantation, and angioplasty) could be treated both acutely and prophylactically using inhibitors of vascular leakage, especially as in the case of Src inhibitors.

Since the activation and perhaps over-expression of Src has been implicated in cancer, osteoporosis, stroke, myocardial infarction, and vascular leak, among others, a small molecule inhibitor of c-Src can be beneficial for the treatment of several disease states.

### SUMMARY

The present invention provides methods of use for certain chemical compounds such as kinase inhibitors for treatment of various diseases, disorders, and pathologies, for example, cancer, and vascular disorders, such as myocardial infarction (MI), stroke, or ischemia.

The pyrimidine compounds described in this invention may be beneficial for treatment of the diseases where disorders affect cell motility, adhesion, and cell cycle progression, and in addition, diseases with related hypoxic conditions, osteoporosis and conditions, which result from or are related to increases in vascular permeability, inflammation or respiratory distress, tumor growth, invasion, angiogenesis, metastases and apoptosis.

According to the embodiments of the invention, some examples of kinase inhibitors that can be used to bring about beneficial therapeutic results include inhibitors of Src kinase.

According to one embodiment of the invention, compounds having structure (A) are provided.

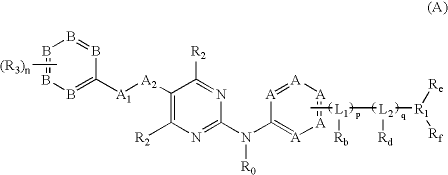

In structure (A), each of A can be, independently, one of CH, N, NH, O, S, or a part of a ring fusion to form a second ring, wherein the second ring can be an aromatic, a heteroaromatic, a bicyclic aromatic, or a bicyclic aromatic heterocyclic ring;

each of B can be, independently CH, or a part of a ring fusion to form a second ring, wherein the second ring can be an aromatic, a bicyclic aromatic, or a bicyclic with only the first ring being aromatic;

A₁ can be one of NRₐ, C(O), S(O), S(O)₂, P(O)₂, O, S, or CRₐ, where R can be one of H, lower alkyl, branched alkyl, hydroxyalkyl, aminoalkyl, thioalkyl, alkylhydroxyl, alklythiol, or alkylamino, and wherein a=1, if A₁ is NRₐ, and a=2, if A₁ is Ca;

A₂ can be one of NR, C(O), S(O), S(O)₂, P(O)₂, O, or S, with the proviso that the connectivity between A₁ and A₂ is chemically correct;

R₀ can be one of H, lower alkyl, or branched alkyl;

L₁ can be one of a bond, O, S, C(O), S(O), S(O)₂, NRₐ, C₁-C₆ alkyl; L₂ can be one of a bond, O, S, C(O), S(O), S(O)₂, C₁-C₆, NRₐ; or L₁ and L₂ taken together can be a bond;

each of R_{b}, R_{d}, Rₑ, R_{f} either is absent or is independently one of H, C₁-C₆ alkyl, cycloalkyl, branched alkyl, hydroxy alkyl, aminoalkyl, thioalkyl, alkylhydroxyl, alkklythiol, or alkylamino;

each of p, q, m, r is independently an integer having value from 0 to 6;

R_{b} and R_{d} taken together can be one of (CH₂)ₘ, (CH₂)ᵣ—S—(CH₂)ₘ, (CH₂)ᵣ—SO—(CH₂)ₘ, CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ;

or R_{d} and R_{f} taken together can be one of (CH₂)ₘ, (CH₂)ᵣ—S—(CH₂)ₘ, (CH₂)ᵣ—SO—(CH₂)ₘ, (CH₂)ᵣ, SO₂—(CH₂)ₘ, (CH₂)ᵣ—NRₐ—(CH₂)ₘ, or (CH₂)ᵣ—O—(CH₂)ₘ; or

R_{b} and R_{f} taken together can be one of (CH₂)ₘ, (CH₂)ᵣ—S—(CH₂)ₘ, (CH₂)ᵣ—SO—(CH₂)ₘ (CH₂)ᵣ—SO₂—(CH₂)ₘ, (CH₂)ᵣ—NRₐ—(CH₂)ₘ, or (CH₂)ᵣ—O—(CH₂)ₘ; or

R_{d} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ;

R₁ can be one of (CRₐ)ₘ, O, N, S, C(O)(O)R′, C(O)N(R′)₂, SO₃R′, OSO₂R′, SO₂R′, SOR′, PO₄R′, OPO₂R′, PO₃R′, PO₂R′, or a 3-6 membered heterocycle with one or more heterocyclic atoms, wherein R′ can be one of hydrogen, lower alkyl, alkyl-hydroxyl, or can form a closed 3-6 membered heterocycle with one or more heterocyclic atoms, branched alkyl, branched alkyl hydroxyl, where each R′ is independent in case there is more than one R′;

R₂ can be one of hydrogen, alkyl, branched alkyl, phenyl, substituted phenyl, halogen, alkylamino, alkyloxo, CF₃, sulfonamido, substituted sulfonamido, alkyoxy, thioalkyl, sulfonate, sulfonate ester, phosphate, phosphate ester, phosphonate, phosphonate ester, carboxo, amido, ureido, substituted carboxo, substituted amido, substituted ureido, or 3-6 membered heterocycle with one or more hetrocyclic atoms, with the further proviso that either one or two substituents R₂ can be present in the ring, and if more than one substituent R₂ are present, each of the substituents can be the same or different;

R₃ can be one of hydrogen, alkyl, branched alkyl, alkoxy, halogen, CF₃, cyano, substituted alkyl, hydroxyl, alklylhydroxyl, thiol, alkylthiol, thioalkyl, amino, or aminoalkyl; and

n is an integer that can have value between 1 and 5, with the further proviso that if n≧2, then each group R₃ is independent of the other groups R₃.

In another embodiment, there are provided pharmaceutical compositions including at least one compound of structure (A) and a pharmaceutically acceptable carrier therefore.

In yet another embodiment, there are provided articles of manufacture including packaging material and a pharmaceutical composition contained within the packaging material, wherein the packaging material includes a label which indicates that the pharmaceutical composition can be used for treatment of disorders associated with compromised vasculostasis and wherein the pharmaceutical composition includes at least one compound of structure (A).

In another embodiment, there are provided articles of manufacture including packaging material and a pharmaceutical composition contained within the packaging material, wherein the packaging material includes a label which indicates that the pharmaceutical composition can be used for treatment of disorders associated with vascular permeability leakage or compromised vasculostasis selected from myocardial infarction, stroke, congestive heart failure, an ischemia or reperfusion injury, cancer, arthritis or other arthropathy, retinopathy or another ophthalmological disease, e.g., macular degeneration, autoimmune disease, vascular leakage syndrome, inflammatory disease, edema, transplant rejection, burn, or acute or adult respiratory distress syndrome (ARDS) and wherein the pharmaceutical composition includes at least one compound of structure (A).

In another embodiment, there are provided methods of treating a disorder associated with compromised vasculostasis, including the administration of a therapeutically effective amount of at least one compound of structure 1 or pharmaceutically acceptable salts, hydrates, solvates, crystal forms and individual diastereomers thereof, to a subject in need of such treatment.

In yet another embodiment, there are provided methods of treating a disorder associated with compromised vasculostasis including the administration of a therapeutically effective amount of at least one compound of structure (A), or pharmaceutically acceptable salts, hydrates, solvates, crystal forms and individual diastereomers thereof, in combination with an anti-inflammatory, chemotherapeutic agent, immunomodulatory agent, therapeutic antibody or a protein kinase inhibitor, to a subject in need of such treatment.

In another embodiment, there are provided methods of treating a subject having or at risk of having myocardial infarction including administering to the subject a therapeutically effective amount of at least one compound of structure (A), thereby treating the subject.

In another embodiment, there are provided methods of treating a subject having or at risk of having vascular leakage syndrome (VLS) including administering to the subject a therapeutically effective amount of at least one compound of structure (A), thereby treating the subject.

In another embodiment, there are provided methods of treating a subject having or at risk of having cancer including administering to the subject a therapeutically effective amount of at least one compound of structure (A), thereby treating the subject.

In another embodiment, there are provided methods of treating a subject having or at risk of having stroke including administering to the subject a therapeutically effective amount of at least one compound of structure (A), thereby treating the subject.

In another embodiment, there are provided methods of treating a subject having or at risk of having ARDS including administering to the subject a therapeutically effective amount of at least one compound of structure (A), thereby treating the subject.

In another embodiment, there are provided methods of treating a subject having or at risk of having burns including administering to the subject a therapeutically effective amount of at least one compound of structure (A), thereby treating the subject.

In another embodiment, there are provided methods of treating a subject having or at risk of having arthritis including administering to the subject a therapeutically effective amount of at least one compound of structure (A), thereby treating the subject.

In another embodiment, there are provided methods of treating a subject having or at risk of having edema including administering to the subject a therapeutically effective amount of at least one compound of structure (A), thereby treating the subject.

In another embodiment, there are provided methods of treating a subject having or at risk of having vascular leakage syndrome (VLS) including administering to the subject a therapeutically effective amount of at least one compound of structure (A), thereby treating the subject.

In another embodiment, there are provided methods of treating a subject having or at risk of having retinopathy or another ophthalmological disease including administering to the subject a therapeutically effective amount of at least one compound of structure (A), thereby treating the subject.

In another embodiment, there are provided methods of treating a subject having or at risk of having ischemic or reperfusion related tissue injury or damage, including administering to the subject a therapeutically effective amount of at least one compound of structure (A), thereby treating the subject.

In another embodiment, there are provided methods of treating a subject having or at risk of having an autoimmune disease, including administering to the subject a therapeutically effective amount of at least one compound of structure (A), thereby treating the subject.

In another embodiment, there are provided methods of treating a subject having or at risk of having transplant rejection, including administering to the subject a therapeutically effective amount of at least one compound of structure (A), thereby treating the subject.

In another embodiment, there are provided methods of treating a subject having or at risk of having inflammatory disease, including administering to the subject a therapeutically effective amount of at least one compound of structure (A), thereby treating the subject.

In another embodiment, there are provided processes for making a pharmaceutical composition including combining a combination of at least one compound of structure (A) or its pharmaceutically acceptable salts, hydrates, solvates, crystal forms salts and individual diastereomers thereof and a pharmaceutically acceptable carrier.

### DETAILED DESCRIPTION

A. Terms and Definitions.

The following terminology and definitions apply as used in the present application, generally in conformity with the terminology recommended by the International Union of Pure and Applied Chemistry (IUPAC):

The term “heteroatom” refers to any atom other than carbon, for example, N, O, or S.

The term “aromatic” refers to a cyclically conjugated molecular entity with a stability, due to delocalization, significantly greater than that of a hypothetical localized structure, such as the Kekule structure.

The term “heterocyclic,” when used to describe an aromatic ring, refers to the aromatic rings containing at least one heteroatom, as defined above.

The term “heterocyclic,” when not used to describe an aromatic ring, refers to cyclic (i.e., ring-containing) groups other than aromatic groups, the cyclic group being formed by between 3 and about 14 carbon atoms and at least one heteroatom described above.

The term “substituted heterocyclic” refers, for both aromatic and non-aromatic structures, to heterocyclic groups further bearing one or more substituents described below.

The term “alkyl” refers to a monovalent straight or branched chain hydrocarbon group having from one to about 12 carbon atoms, for example, methyl, ethyl, n-propyl, iso-propyl, n-butyl, iso-butyl, tert-butyl, n-pentyl (also known as n-amyl), n-hexyl, and the like. The term “lower alkyl” refers to alkyl groups having from 1 to about 6 carbon atoms.

The term “substituted alkyl” refers to alkyl groups further bearing one or more substituents such as hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, aldehyde, acyl, oxyacyl, carboxyl, sulfonyl, sulfonamide, sulfuryl, and the like.

The term “alkenyl” refers to straight-chained or branched hydrocarbyl groups having at least one carbon-carbon double bond, and having between about 2 and about 12 carbon atoms, and the term “substituted alkenyl” refers to alkenyl groups further bearing one or more substituents described above.

The term “alkynyl” refers to straight-chained or branched hydrocarbyl groups having at least one carbon-carbon triple bond, and having between about 2 and about 12 carbon atoms, and the term “substituted alkynyl” refers to alkynyl groups further bearing one or more substituents described above.

The term “aryl” refers to aromatic groups having between about 5 and about 14 carbon atoms and the term “substituted aryl” refers to aryl groups further bearing one or more substituents described above.

The term “heteroaryl” refers to aromatic rings, where the ring structure is formed by between 3 and about 14 carbon atoms and by at least one heteroatom described above, and the term “substituted heteroaryl” refers to heteroaryl groups further bearing one or more substituents described above.

The term “alkoxy” refers to the moiety —O-alkyl, wherein alkyl is as defined above, and the term “substituted alkoxy” refers to alkoxy groups further bearing one or more substituents described above.

The term “cycloalkyl” refers to alkyl groups having between 3 and about 8 carbon atoms arranged as a ring, and the term “substituted cycloalkyl” refers to cycloalkyl groups further bearing one or more substituents described above.

The term “alkylaryl” refers to alkyl-substituted aryl groups and the term “substituted alkylaryl” refers to alkylaryl groups further bearing one or more substituents described above.

The term “arylalkyl” refers to aryl-substituted alkyl groups and the term “substituted arylalkyl” refers to arylalkyl groups further bearing one or more substituents described above.

The term “arylalkenyl” refers to aryl-substituted alkenyl groups and the term “substituted arylalkenyl” refers to arylalkenyl groups further bearing one or more substituents described above.

The term “arylalkynyl” refers to aryl-substituted alkynyl groups and the term “substituted arylalkynyl” refers to arylalkynyl groups further bearing one or more substituents described above.

The term “arylene” refers to divalent aromatic groups having between 5 and about 14 carbon atoms and the term “substituted arylene” refers to arylene groups further bearing one or more substituents described above.

The term “kinase” refers to any enzyme that catalyzes the addition of phosphate groups to a protein residue; for example, serine and threonine kinases catalyze the addition of phosphate groups to serine and threonine residues.

The terms “Src kinase,” “Src kinase family,” and “Src family” refer to the related homologs or analogs belonging to the mammalian family of Src kinases, including, for example, c-Src, Fyn, Yes and Lyn kinases and the hematopoietic-restricted kinases Hck, Fgr, Lck and Blk.

The terms “Src kinase signaling pathway,” and “Src cascade” refer to both the upstream and downstream components of the Src signaling cascade.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

The term “antibody” refers to intact molecules of polyclonal or monoclonal antibodies, as well as fragments thereof, such as Fab and F(ab′)₂, Fv and SCA fragments which are capable of binding an epitopic determinant.

The term “vasculostasis” refers to the maintenance of the homeostatic vascular functioning leading to the normal physiologic functioning.

The term “vasculostatic agents” refers to agents that seek to address conditions in which vasculostasis is compromised by preventing the loss of or restoring or maintaining vasculostasis.

### B. Embodiments of the Invention

According to an embodiment of the invention, compounds having the structure (A) are provided for treatment of various diseases, disorders, and pathologies.

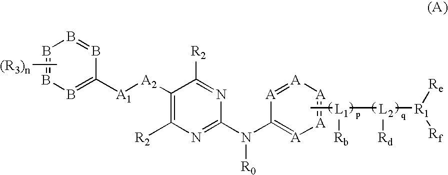

In structure (A), each of A can be, independently, one of CH, N, NH, O, S, or a part of a ring fusion to form a second ring, wherein the second ring can be an aromatic, a heteroaromatic, a bicyclic aromatic, or a bicyclic aromatic heterocyclic ring.

In structure (A), each of B can be, independently CH, or a part of a ring fusion to form a second ring, wherein the second ring can be an aromatic, a bicyclic aromatic, or a bicyclic with only the first ring being aromatic.

In structure (A), A₁ can be one of NRₐ, C(O), S(O), S(O)₂, P(O)₂, O, S, or CRₐ, where R can be one of H, lower alkyl, branched alkyl, hydroxyalkyl, aminoalkyl, thioalkyl, alkylhydroxyl, alklythiol, or alkylamino, and wherein a=1, if A₁ is NRₐ, and a=2, if A₁ is CRₐ.

In structure (A), A₂ can be one of NR, C(O), S(O), S(O)₂, P(O)₂, O, or S, with the proviso that the connectivity between A₁ and A₂ is chemically correct.

In structure (A), R₀ can be one of H or lower alkyl.

In structure (A), L₁ can be one of a bond, O, S, C(O), S(O), S(O)₂, NRₐ, C₁-C₆ alkyl; L₂ can be one of a bond, O, S, C(O), S(O), S(O)₂, C₁-C₆, NRₐ; or L₁ and L₂ taken together can be a bond.

In structure (A), each of R_{b}, R_{d}, Rₑ, R_{f} either is absent or is independently one of H, C₁-C₆ alkyl, cycloalkyl, branched alkyl, hydroxy alkyl, aminoalkyl, thioalkyl, alkylhydroxyl, alkklythiol, or alkylamino. In structure (A), each of p, q, m, r is independently an integer having value from 0 to 6.

In structure (A), Rb and Rd taken together can be one of (CH₂)ₘ, (CH₂)ᵣ—S—(CH₂)ₘ, (CH₂)ᵣ—SO—(CH₂)ₘ, CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ, NRₐ—(CH₂)ₘ, or (CH₂)ᵣ—O—(CH₂)ₘ;

or R_{d} and R_{f} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and R_{f} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{d} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—Nₐ—(CH₂)ₘ, and (CH₂)ᵣ—O—(CH₂)ₘ.

In structure (A), R₁ can be one of (CRₐ)ₘ, O, N, S, C(O)(O)R′, C(O)N(R′)₂, SO₃R′, OSO₂R′, SO₂R′, SOR′, PO₄R′, OPO₂R′, PO₃R′, PO₂R′, or a 3-6 membered heterocycle with one or more heterocyclic atoms, wherein R′ can be one of hydrogen, lower alkyl, alkyl-hydroxyl, or can form a closed 3-6 membered heterocycle with one or more heterocyclic atoms, branched alkyl, branched alkyl hydroxyl, where each R′ is independent in case there is more than one R′.

In structure (A), R₂ can be one of hydrogen, alkyl, branched alkyl, phenyl, substituted phenyl, halogen, alkylamino, alkyloxo, CF₃, sulfonamido, substituted sulfonamido, alkyoxy, thioalkyl, sulfonate, sulfonate ester, phosphate, phosphate ester, phosphonate, phosphonate ester, carboxo, amido, ureido, substituted carboxo, substituted amido, substituted ureido, or 3-6 membered heterocycle with one or more hetrocyclic atoms, with the further proviso that either one or two substituents R₂ can be present in the ring, and if more than one substituent R₂ are present, each of the substituents can be the same or different.

In structure (A), R₃ can be one of hydrogen, alkyl, branched alkyl, alkoxy, halogen, CF₃, cyano, substituted alkyl, hydroxyl, alklylhydroxyl, thiol, alkylthiol, thioalkyl, amino, or aminoalkyl.

In structure (A), n is an integer that can have value between 1 and 5, with the further proviso that if n≧2, then each group R₃ is independent of the other groups R₃.

One class of exemplary compounds described by structure (A) that can be used includes compounds I through LX shown below:

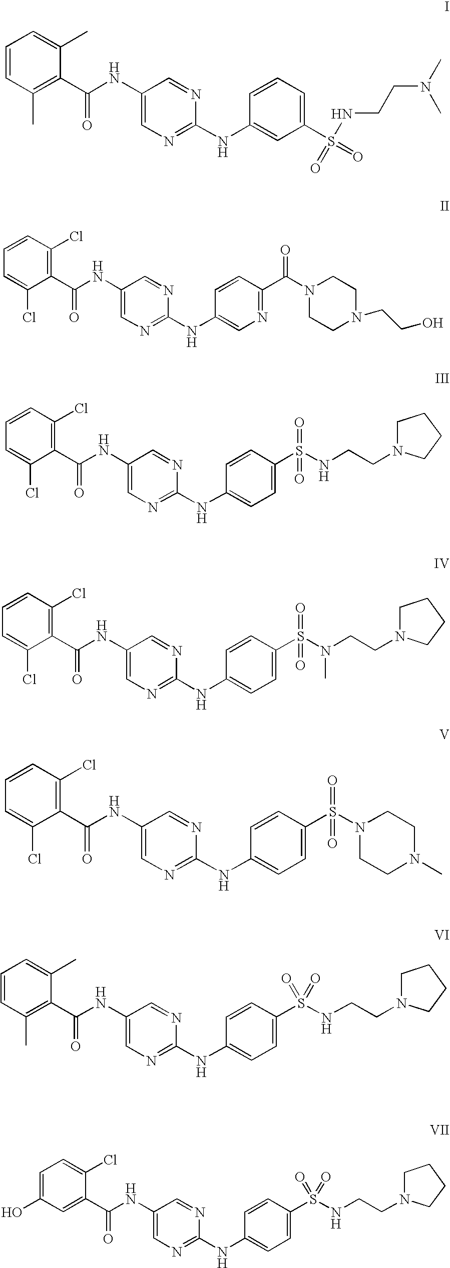

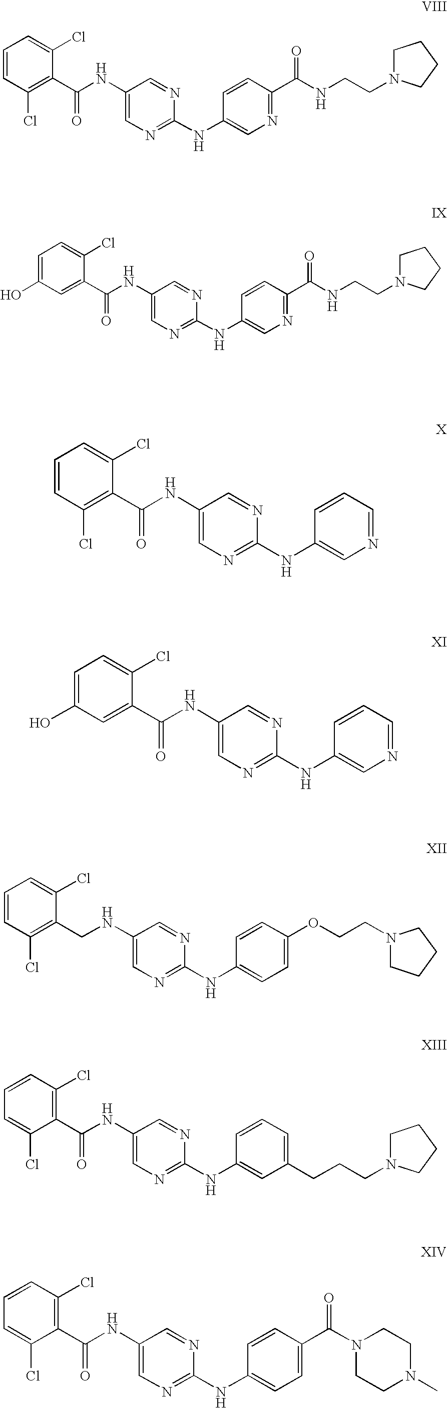

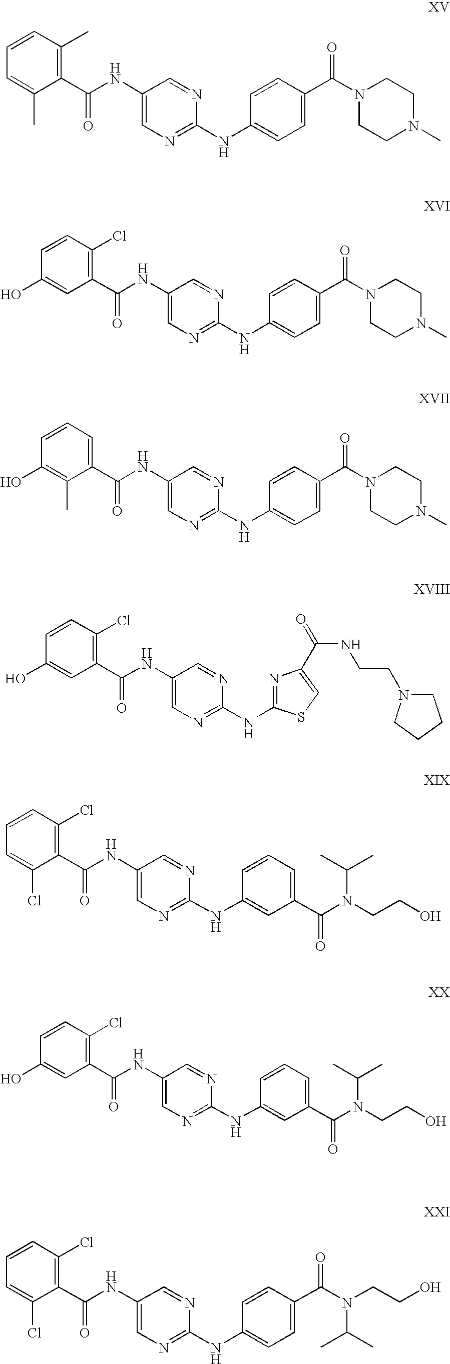

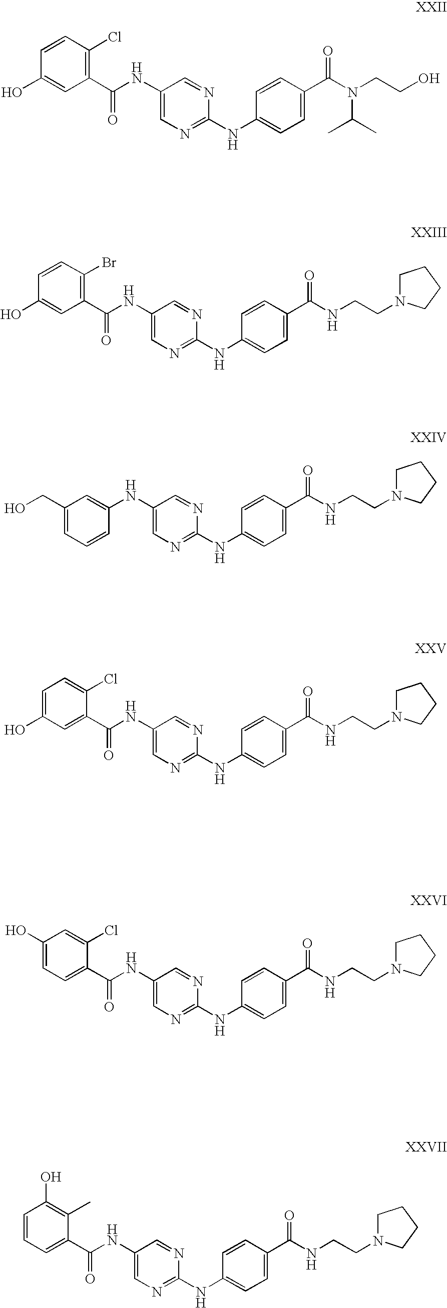

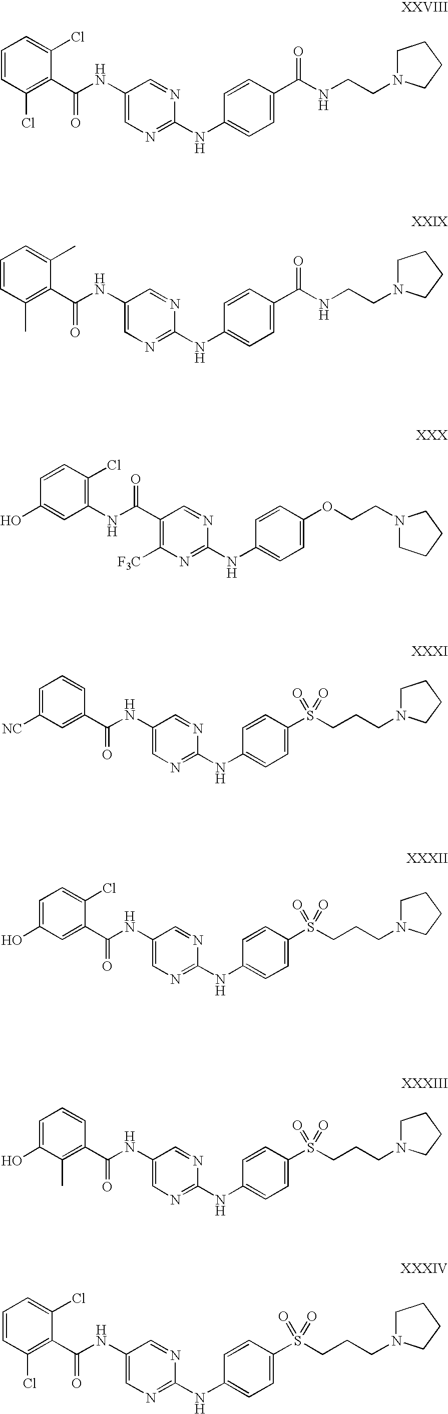

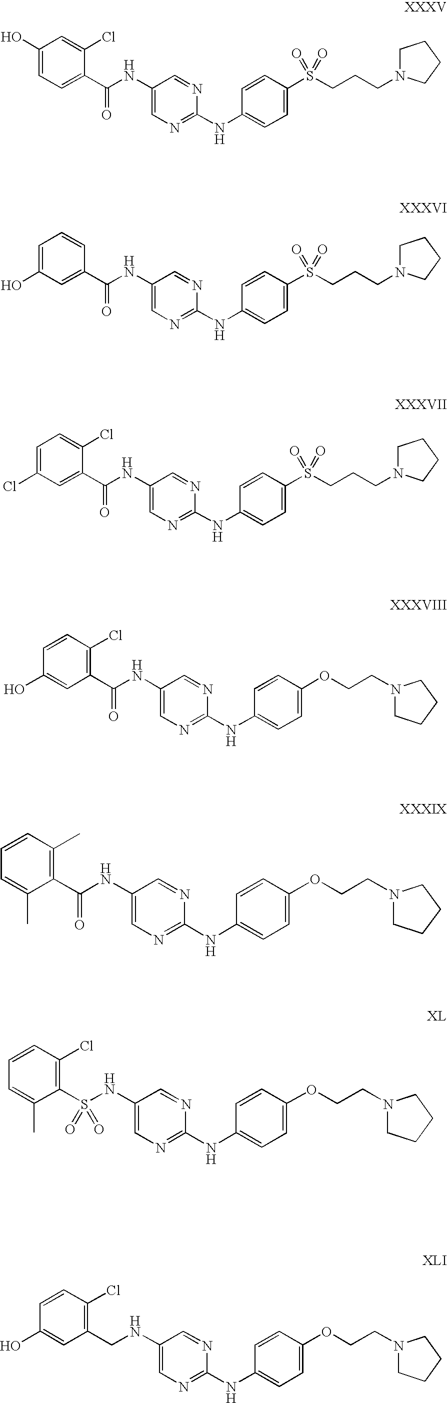

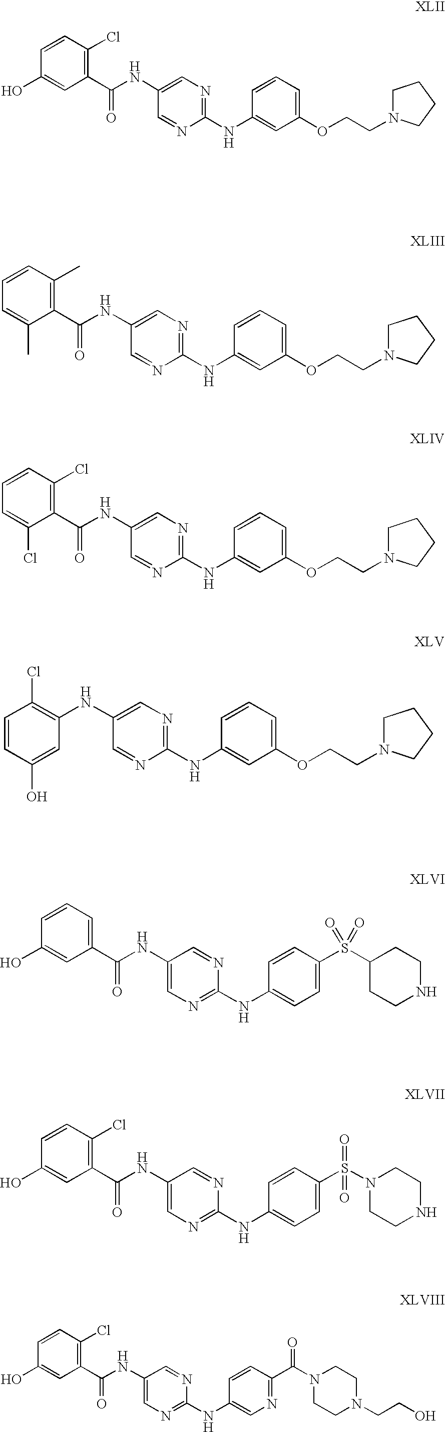

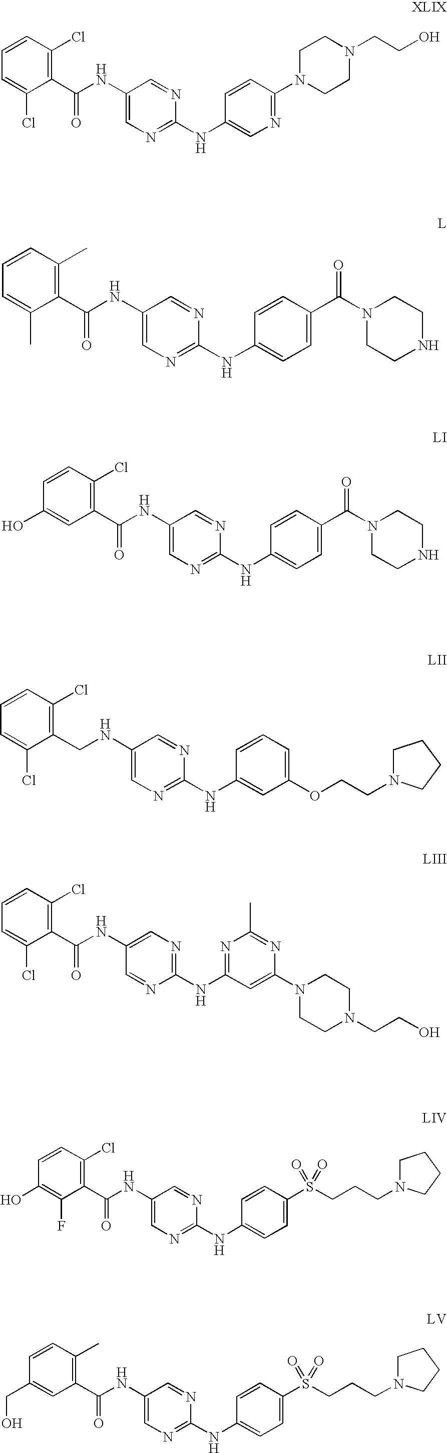

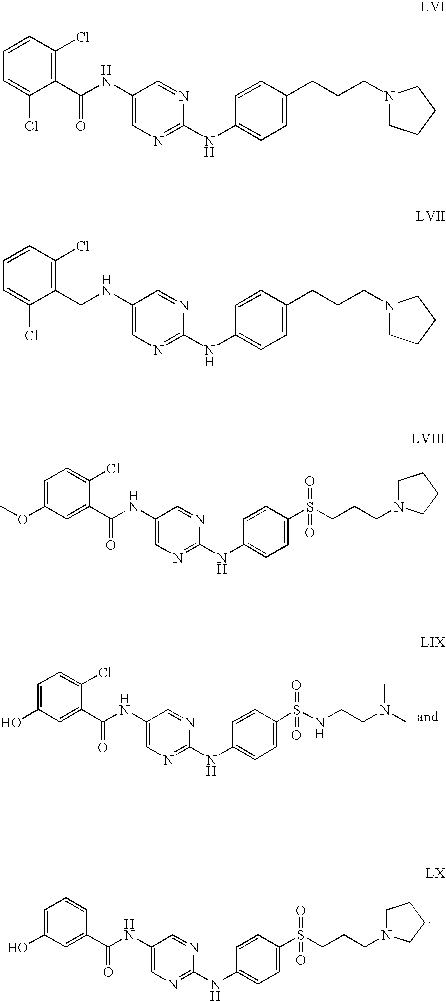

The methods, compounds, and compositions of the present invention, either when administered alone or in combination with other agents (e.g., chemotherapeutic agents or protein therapeutic agents described below) are useful in treating a variety of disorders associated with compromised vasculostasis and other disorders, including but not limited to: stroke, cardiovascular disease, myocardial infarction, congestive heart failure, cardiomyopathy, myocarditis, ischemic heart disease, coronary artery disease, cardiogenic shock, vascular shock, pulmonary hypertension, pulmonary edema (including cardiogenic pulmonary edema), cancer, pleural effusions, rheumatoid arthritis, diabetic retinopathy, retinitis pigmentosa, and retinopathies, including diabetic retinopathy (DR) and retinopathy of prematurity, inflammatory diseases, restenosis, edema (including edema associated with pathologic situations such as cancers, or edema induced by medical interventions such as chemotherapy, or diabetic macular edema (DME)), asthma, acute or adult respiratory distress syndrome (ARDS), lupus, vascular leakage, transplant (such as organ transplant, acute transplant or heterograft or homograft (such as is employed in burn treatment)) rejection; protection from ischemic or reperfusion injury such as ischemic or reperfusion injury incurred during organ transplantation, transplantation tolerance induction; ischemic or reperfusion injury following angioplasty; arthritis (such as rheumatoid arthritis, psoriatic arthritis or osteoarthritis); multiple sclerosis; inflammatory bowel disease, including ulcerative colitis and Crohn's disease; lupus (systemic lupus crythematosis); graft vs. host diseases; T-cell mediated hypersensitivity diseases, including contact hypersensitivity, delayed-type hypersensitivity, and gluten-sensitive enteropathy (Celiac disease); Type 1 diabetes; psoriasis; contact dermatitis (including that due to poison ivy); Hashimoto's thyroiditis; Sjogren's syndrome; Autoimmune Hyperthyroidism, such as Graves'. disease; Addison's disease (autoimmune disease of the adrenal glands); autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; cancers, including those where kinases such as Src-family kinases are activated or overexpressed, such as colon carcinoma and thymoma, or cancers where kinase activity facilitates tumor growth or survival; glomerulonephritis, serum sickness; uticaria; allergic diseases such as respiratory allergies (asthma, hayfever, allergic rhinitis) or skin allergies; mycosis fungoides; acute inflammatory responses (such as acute or adult respiratory distress syndrome and ischemia/reperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic schlerosis; morphea; peripheral limb ischemia and ischemic limb disease; bone disease such as osteoporosis, osteomalacia, hyperparathyroidism, Paget's disease, and renal osteodystrophy; vascular leak syndromes, including vascular leak syndromes induced by chemotherapies or immunomodulators such as IL-2; spinal cord and brain injury or trauma; glaucoma; retinal diseases, vitroretinal diseases, including macular degeneration such as age-related nmacular degeneration (AMD), including dry AMD, or another ophthalmological disease; pancreatitis; vasculatides, including vasculitis, Kawasaki disease, thromboangiitis obliterans, Wegener's granulomatosis, and Behcet's disease; scleroderma; preeclampsia; thalassemia; Kaposi's sarcoma; von Hippel Lindau disease; and the like. The compounds, compositions, and methods of the present invention may be useful in reducing the risk of progression of the ophthalmological disease.

The compounds, compositions, and methods of the present invention may be useful in inhibiting the Fc gamma induced respiratory burst response in neutrophils, and may also be useful in inhibiting the Fc gamma dependent production of TNF alpha. The ability to inhibit Fc gamma receptor dependent neutrophil, monocyte and macrophage responses may result in additional anti-inflammatory activity for the compounds employed in invention methods. This activity may be used, for example, in the treatment of inflammatory diseases, such as arthritis or inflammatory bowel disease. The compounds, compositions and methods of the present invention may also be useful in the treatment of autoimmune glomerulonephritis and other instances of glomerulonephritis induced by deposition of immune complexes in the kidney that trigger Fc gamma receptor responses and which can lead to kidney damage.

The compounds, compositions, and methods of the present invention may be also used to inhibit the Fc epsilon induced degranulation responses. The ability to inhibit Fc epsilon receptor dependent mast cell and basophil responses may result in additional anti-inflammatory activity for the present compounds beyond their effect on T cells.

The present invention also provides articles of manufacture comprising packaging material and a pharmaceutical composition contained within the packaging material, wherein the packaging material comprises a label which indicates that the pharmaceutical composition can be used for treatment of disorders and wherein the pharmaceutical composition comprises a compound according to the present invention. Thus, in one aspect, the invention provides a pharmaceutical composition including a therapeutic agent and a compound of the invention, wherein the compound is present in a concentration effective to reduce vascular leakage associated with indications or therapeutic agents which have vascular leak as a side effect. For example, administration of a compound of the invention can be in conjunction with IL-2, immunotoxins, antibodies or chemotherapeutics. In these cases, IL-2, immunotoxin, antibody or chemotherapeutic concentration can be determined by one having ordinary skill in the art according to standard treatment regimen or, for example, as determined by an in vivo animal assay.

The present invention also provides pharmaceutical compositions comprising IL-2, immunotoxin, antibody or chemotherapeutic and at least one invention compound in an amount effective for inhibiting vascular permeability, and a pharmaceutically acceptable vehicle or diluent. The compositions of the present invention may contain other therapeutic agents, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques known in the art of pharmaceutical formulation.

The compounds of the invention may be formulated into therapeutic compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the invention include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference. In addition, polymorphs of the invention compounds are included in the present invention.

Pharmaceutical compositions of the invention may be administered by any suitable means, for example, orally, such as in the form of tablets, capsules, granules or powders; sublingually; buccally; parenterally, such as by subcutaneous, intravenous, intramuscular, intrathecal, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The pharmaceutical compositions for the administration of the compounds of this embodiment either alone or in combination with IL-2, immunotoxin, antibody or chemotherapeutic may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gel capsules, such as soft gelatin capsules, wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Also useful as a solubilizer is polyethylene glycol, for example. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a parenterally-acceptable diluent or solvent or cosolvent or complexing agent or dispersing agent or excipient or combination thereof, for example 1,3-butanediol, polyethylene glycols, polypropylene glycols, ethanol or other alcohols, povidones, various brands of TWEEN surfactant, sodium dodecyl sulfate, sodium deoxycholate, dimethylacetamide, polysorbates, poloxamers, cyclodextrins, lipids, and excipients such as inorganic salts (e.g., sodium chloride), buffering agents (e.g., sodium citrate, sodium phosphate), and sugars (e.g., saccharose and dextrose). Among the acceptable vehicles and solvents that may be employed are water, dextrose solutions, Ringer's solutions and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. For opthalmological applications, the pharmaceutical compositions can be administered to the back of the eye, intravitreally, or periocularly.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. For opthalmological applications, the pharmaceutical compositions can be formulated and administered in the form of eye-drops.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In one aspect, the invention compounds are administered in combination with an anti-inflammatory agent, antihistamines, chemotherapeutic agent, immunomodulator, therapeutic antibody or a protein kinase inhibitor, e.g., a tyrosine kinase inhibitor, to a subject in need of such treatment. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase, and hydroxyurea, etc.; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-.alpha., etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

The pharmaceutical compositions and methods of the present invention may further comprise other therapeutically active compounds as noted herein which are usually applied in the treatment of the above mentioned pathological conditions. Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-a inhibitors such as tenidap, anti-TNF antibodies or soluble TNF receptor, and rapamycin (sirolimus or Rapamune) or derivatives thereof.

Other agents that may be administered in combination with invention compounds include protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

When other therapeutic agents are employed in combination with the compounds of the present invention they may be used for example in amounts as noted in the Physician Desk Reference (PDR) or as otherwise determined by one having ordinary skill in the art.

In the treatment or prevention of conditions which involve compromised vasculostasis an appropriate dosage level can generally be between about 0.01 and about 500 mg per 1 kg of patient body weight per day which can be administered in single or multiple doses. For example, the dosage level can be between about 0.01 and about 250 mg/kg per day; more narrowly, between about 0.5 and about 100 mg/kg per day. A suitable dosage level can be between about 0.01 and about 250 mg/kg per day, between about 0.05 and about 100 mg/kg per day, or between about 0.1 and about 50 mg/kg per day, or about 1.0 mg/kg per day. For example, within this range the dosage can be between about 0.05 and about 0.5 mg/kg per day, or between about 0.5 and about 5 mg/kg per day, or between about 5 and about 50 mg/kg per day. For oral administration, the compositions can be provided in the form of tablets containing between about 1.0 and about 1,000 mg of the active ingredient, for example, about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1,000.0 mg of the active ingredient for the symptomatic adjustment of the dosage to the patient to be treated. The compounds can be administered on a regimen of 1 to 4 times per day, such as once or twice per day. There may be a period of no administration followed by another regimen of administration. Preferably, administration of the compound is closely associated with the schedule of IL-2 administration. For example, administration can be prior to, simultaneously with or immediately following IL-2 administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Compounds of the present invention can be used, alone or in combination with an effective amount of a therapeutic antibody (or therapeutic fragment thereof), a chemotherapeutic or an immunotoxic agent, for treatment of tumors. While doxorubicin, docetaxel, or taxol are described in the present application as illustrative examples of chemotherapeutic agents, it should be understood that the invention includes combination therapy including a compound of the invention, including but not limited to vasculostatic agents, such as tyrosine, serine or threonine kinase inhibitors, for example, Src-family inhibitors, and any chemotherapeutic agent or therapeutic antibody.

### C. EXAMPLES

The following examples are provided to further illustrate the advantages and features of the present invention, but are not intended to limit the scope of the invention.

### Example 1

### General Methods

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated. Analytical thin layer chromatography (TLC) was carried out on Merck Kieselgel 60 F₂₅₄ plates with visualization by ultraviolet and/or anisaldehyde, potassium permanganate or phosphomolybdic acid dips. Reverse-phase HPLC chromatography was carried out on Gilson 215 liquid handler equipped with Waters SymmetryShield™ RP18 7 μm (40×100 mm) Prep-Pak cartridge. Mobile phase consisted of standard acetonitrile (ACN) and DI Water, each with 0.1% TFA added. Purification was carried out at a flow rate of 40 mL/min. NMR spectra: ¹H Nuclear magnetic resonance spectra were recorded at 500 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken directly from the spectra and are uncorrected. Low resolution mass spectra: Electrospray (ES+) ionization was used. The protonated parent ion (M+H) or fragment of highest mass is quoted. Analytical gradient consisted of 10% ACN in water ramping up to 100% ACN over 5 minutes unless otherwise stated.

### Example 2

### N-(2-Dimethylamino-ethyl)-3-(5-nitro-pyrimidin-2-ylamino)-benzenesulfonamide (1)

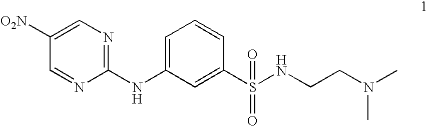

5-nitro-pyrimidin-2-ylamine (1.11 mmol, 1.0 equiv), Pd₂(dba)₃ (0.111 mmol, 0.1 equiv), Cs₂CO₃ (3.33 mmol, 3.0 equiv), Xantphos (0.222 mmol, 0.2 equiv), and 3-bromo-N-(2-dimethylamino-ethyl)-benzenesulfonamide (1.67 mmol, 1.5 equiv) were dissolved in 6 mL dioxane and purged of air using vacuum. The reaction mixture was placed under an argon atmosphere and refluxed at 100° C. for 18 h. Palladium and Cs₂CO₃ were filtered through Celite, and then extracted using EtOAc, saturated NaHCO₃ and brine. The organic phase was dried (MgSO₄) and concentrated under reduced pressure. The residue was precipitated with EtOAc/Hexanes (1:5 v/v) to afford the title compound as a tan solid (216 mg, 22%).

### Example 3

### 3-(5-Amino-pyrimidin-2-ylamino)-N-(2-dimethylamino-ethyl)-benzenesulfonamide (2)

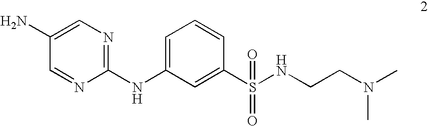

Compound 1 (0.464 mmol, 1.0 equiv) was dissolved in 6 mL of MeOH. Sample evacuated of air was then placed under an argon blanket; Pd/C (10% by wt) added to reaction mixture and sample evacuated of argon was then blanketed with hydrogen. The reaction mixture was stirred at room temperature for 4 h. Product was filtered through Celite to remove palladium and then concentrated under reduced pressure. The residue was purified by flash chromatography on a 5 cm×40 cm column using DCM/MeOH 50:50 as eluent. Pure product was precipitated using MeOH/Et₂O (1:5 v/v) to afford the title compound as a pale yellow solid (43 mg, 28%). MS (ES+): m/z 337 (M+H)⁺ LC retention time: 1.26 min.

### Example 4

### N-{2-[3-(2-Dimethylamino-ethylsulfamoyl)-phenylamino]-Pyrimidin-5-yl}-2,6-dimethyl-benzamide (1)

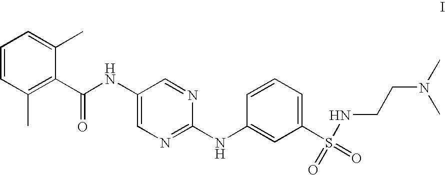

Compound 2 (0.055 mmol, 2.0 equiv) described in Example 3, 2, 6-dimethylbenzoyl chloride (0.030 mmol, 1.0 equiv) and TEA (0.12 mmol, 4.0 equiv) were dissolved in 5 mL toluene. The reaction mixture was refluxed at 111° C. for 18 h under an argon atmosphere. After cooling to room temperature, the reaction was dissolved in DCM and washed with saturated NaHCO₃ and brine. The organic phase was dried (MgSO₄) and concentrated under reduced pressure. Prep HPLC run using a 10-50-75 acetonitrile and water mobile phase to give the title compound as a white solid (6.7 mg, 48%).

R_{f}=0.14 (DCM/MeOH 9:1). ¹H NMR (DMSO-d₆): δ 2.08 (bs, 3H), 2.29 (s, 6H), 2.88 (bs, 3H), 3.32 (smear under water, 6H), 7.13 (d, J=7.7 Hz, 1H), 7.24 (t, J=7.6 Hz, 2H), 7.34 (d, J=8.2 Hz, 1H), 7.47 (t, J=8.0 Hz, 2H), 7.66 (d, J=8.6 Hz, 2H), 7.87 (t, J=8.5 Hz, 2H), 8.36 (s, 1H), 8.84 (s, 2H), 10.02 (s, 1H), 10.51 (s, 1H). MS (ES+): m/z=469 (M+H)⁺. LC retention time: 2.02 min.

### Example 5

### (5-Bromo-pyridin-2-yl)-[4-(2-hydroxy-ethyl)-piperazin-1-yl]-methanone

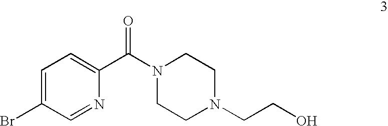

To a solution of 2-piperazin-1-yl-ethanol (1.0 g, 7.7 mmol) and 5-bromo-pyridine-2-carboxylic acid (1.0 g, 5.0 mmol) in dry DMF (0.05-0.2 M) was added HBTU (1.5 mol equiv) and HOBt (1.3 mol equiv) followed by DIEA (3.0 mol equiv). The reaction mixture was stirred at room temperature for 16 h and then diluted with EtOAc. The organic layer was washed with water and brine, dried (MgSO₄). The filtrate was concentrated under reduced pressure and triturated in Hexane/Et₂O (5:1 v/v) to give the title compound as a white solid (1.0 g, 65%).

### Example 6

### [4-(2-Hydroxy-ethyl)-piperazin-1-yl]-[5-(5-nitro-pyrimidin-2-ylamino)-Pyridin-2-yl]methanone (4)

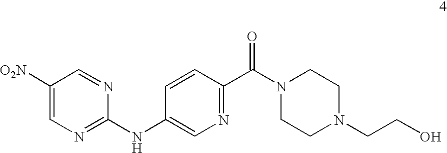

A mixture of 5-nitro-pyrimidin-2-ylamine (0.85 g, 6.1 mmol), compound 3 described in Example 5 (2.5 g, 8.0 mmol), Pd(OAc)₂ (0.4 g, 0.44 mmol), Xantphos (0.5 g, 0.86 mmol) and Cs₂CO₃ (4.0 g, 12 mmol) was suspended in 30 mL of dioxane and refluxed at 100° C. under an argon atmosphere for 18 h. The mixture was allowed to cool to room temperature, filtered and washed with DCM. The filtrate was concentrated and the crude product purified by flash chromatography on silica gel (5% MeOH/DCM to 15% MeOH/DCM) to afford the title compound as a yellow solid (0.9 g, 40%). MS (ES+): m/z=374 (M+H)⁺.

### Example 7

### [5-(5-Amino-pyrimidin-2-ylamino)-pyridin-2-yl]-[4-(2-hydroxy-ethyl)-piperazin-1-yl]-methanone (5)

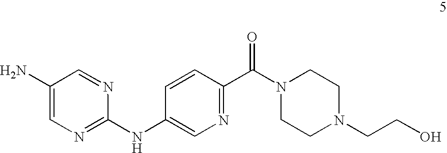

Compound 4 (0.7 g, 1.9 mmol) described in Example 6, dissolved in MeOH (0.05-1.0 M), was evacuated of air and placed under an argon blanket; Pd/C (10% by wt) added. The mixture was evacuated and then refilled with hydrogen and stirred at room temperature for 4 h. The product was filtered through Celite, washed with MeOH and concentrated under reduced pressure to afford the title compound as a white solid. The crude amino-compound was used in the next step without purification. MS (ES+): m/z=344 (M+H)⁺.

### Example 8

### 2,6-Dichloro-N-(2-{6-[4-(2-hydroxy-ethyl)-piperazine-1-carbonyl]-pyridin-3-ylamino}-pyrimidin-5-yl)-benzamide (II)

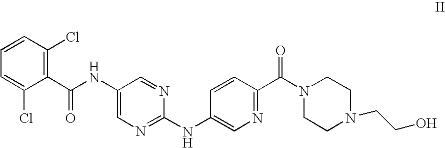

Compound 5 (0.292 mmol, 1.0 equiv) described in Example 7, and 2,6-dichlorobenzoyl chloride (0.437 mmol, 1.5 equiv) were dissolved in 8 mL of THF. TEA (0.584 mmol, 2.0 equiv) was combined via syringe and refluxed at 70° C. for 18 h under an argon atmosphere. Solvent concentrated under reduced pressure and residue was suspended in EtOAc and washed with saturated NaHCO₃ and brine. The organic phase was dried (MgSO₄) and concentrated under reduced pressure. The residue was precipitated out using MeOH/Hexanes (1:5 v/v) to afford the title compound as a pale yellow solid (89.0 mg, 60%).

¹H NMR (DMSO-d₆): δ 1.17 (t, J=7.2 Hz, 2H), 2.42 (m, 4H), 3.51 (q, J=11.6 Hz, J=6.0 Hz, 4H), 3.62 (bs, 2H), 7.57 (m, smeared together), 8.32 (dd, J=8.6 Hz, J=2.6 Hz, 1H), 8.84 (s, 2H), 8.88 (d, J=2.6 Hz, 1H), 10.19 (s, 11H), 10.97 (s, 1H) MS (ES+): m/z=516 (M+H)⁺. LC retention time: 1.78 min.

### Example 9

### 4-Bromo-N-(2-pyrrolidin-1-yl-ethyl)-benzenesulfonamide (6)

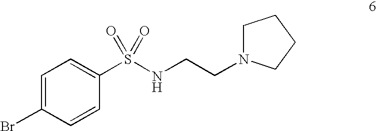

4-Bromo-benzenesulfonyl chloride (3.36 g, 13.1 mmol, 1 equiv) was dissolved in 50 mL DCM and treated with TEA (9.16 mL, 65.7 mmol, 5 equiv). To this, while stirring the solution, was added 2-pyrrolidin-1-yl-ethylamine (3 g, 26.3 mmol, 2 equiv). After 3 hours, reaction was poured onto DCM/water mixture and washed once. The aqueous phase was back extracted once with fresh DCM. Organic phases were combined, washed once with brine and dried over sodium sulfate. Filtration followed by rotary evaporation provided desired product. White needles (3.92 g, 90%). R_{f}=0.35, 10% MeOH/DCM.

### Example 10

### 4-(5-Nitro-pyrimidin-2-ylamino)-N-(2-pyrrolidin-1-yl-ethyl)-benzenesulfonamide (7)

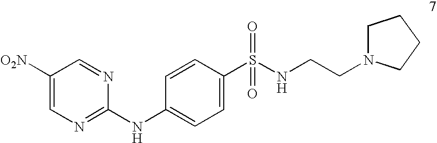

A mixture of 2-amino-5-nitropyrimdin (7.14 mmol, 1.0 equiv), compound 6 (10.71 mmol, 1.5 equiv) described in Example 9, Pd(OAc)₂ (0.357 mmol, 0.05 equiv), Xantphos (0.714 mmol, 0.1 equiv) and potassium-t-butoxide (14.28 mmol, 2.0 equiv) were suspended in 40 mL of dioxane and refluxed at 100° C. under an argon atmosphere for 18 h. The mixture was allowed to cool to room temperature, filtered and washed with DCM. The filtrate was concentrated and the crude product precipitated out using EtOAc/Hexanes (1:5 v/v) to afford the title compound as a yellow solid (1.67 g, 60%). MS (ES+): m/z=393 (M+H)⁺. LC retention time: 1.79 min.

### Example 11

### 4-(5-Amino-pyrimidin-2-ylamino)-N-(2-pyrrolidin-1-yl-ethyl)-benzenesulfonamide (8)

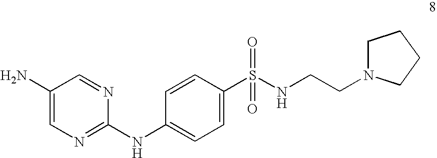

Compound 7 described in Example 10 (4.26 mmol, 1.0 equiv) dissolved in MeOH (0.05-1.0 M) was evacuated of air and placed under an argon blanket; Pd/C (10% by wt) added. The mixture was evacuated and then refilled with hydrogen and stirred at room temperature for 4 h. Filtration through Celite with a MeOH wash, followed by concentration under reduced pressure afford the title compound as a white solid (100 mg, 7%). The crude amino-compound was used in the next step without purification. MS (ES+): m/z=363 (M+H)⁺. LC retention time: 1.34 min.

### Example 12

### 2,6-Dichloro-N-{2-[4-(2-pyrrolidin-1-yl-ethylsulfamoyl)-phenylamino]-pyrimidin-5-yl}-benzamide (III)

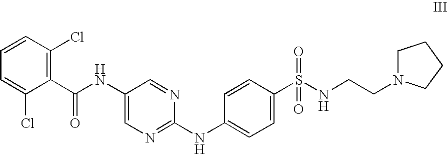

Compound 8 described in Example 11 (0.276 mmol, 1.0 equiv) and 2,6-dichlorobenzoyl chloride (0.414 mmol, 1.5 equiv) were dissolved in 8 mL of THF. TEA (0.552 mmol, 2.0 equiv) was combined via syringe and refluxed at 70° C. for 18 h under an argon atmosphere. Solvent was removed under reduced pressure and residue was suspended in EtOAc and washed with saturated NaHCO₃ and brine. The organic phase was dried (MgSO₄) and concentrated under reduced pressure. The residue was precipitated using MeOH/Et₂O (1:5 v/v) to afford the title compound as a pale yellow solid (26.4 mg, 20%). ¹H NMR (DMSO-d₆): δ 1.71 (bs, 4H), 2.61 (m, 4H), 2.88 (bs, 2H), 3.38 (m, smeared under water), 7.54 (dd, J=7.3 Hz, J=8.9 Hz, 1H), 7.61 (d, J=7.6 Hz, 2H), 7.71 (q, J=7.1 Hz, 2H), 7.94 (m, J=7.2 Hz, 2H), 8.84 (s, 2H), 10.24 (s, 1H), 11.01 (s, 1H). MS (ES+): m/z=535 (M+H)⁺. LC retention time: 2.07 min.

### Example 13

### N-Methyl-4-(5-nitro-pyrimidin-2-ylamino)-N-(2-pyrrolidin-1-yl-ethyl)-benzenesulfonamide (9)

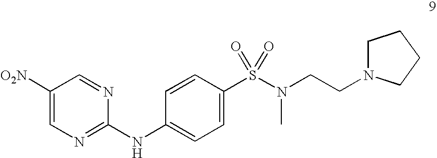

5-Nitro-pyrimidin-2-ylamine (0.964 mmol, 1.0 equiv), 4-bromo-N-methyl-N-(2-pyrrolidin-1-yl-ethyl)-benzenesulfonamide (1.45 mmol, 1.5 equiv), Pd₂(dba)₃ (0.096 mmol, 0.1 equiv), Cs₂CO₃ (2.89 mmol, 3.0 equiv), and Xantphos (0.193 mmol, 0.2 equiv) were dissolved in 25 mL dioxane and purged of air using vacuum. Reaction mixture was placed under an argon atmosphere and refluxed at 100° C. for 18 h. Solvent was filtered through Celite to remove excess palladium and Cs₂CO₃, then extracted using EtOAc, saturated NaHCO₃ and brine. Organic phase dried (MgSO₄) and concentrated under reduced pressure. The residue was dissolved in MeOH and purified using a silica plug (5%-20% MeOH/DCM) to give the title compound as a tan solid (41.6 mg, 11%). MS (ES+): m/z=409 (M+H)⁺. LC retention time: 1.95 min.

### Example 14

### 4-(5-Amino-pyrimidin-2-ylamino)-N-methyl-N-(2-pyrrolidin-1-yl-ethyl)-benzenesulfonamide (10)

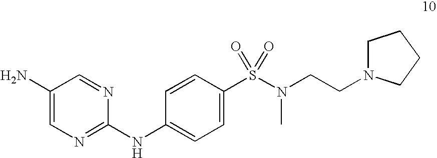

Compound 9 described in Example 13 (0.099 mmol, 1.0 equiv) dissolved in MeOH (0.05-1.0 M) was evacuated of air and placed under an argon blanket; Pd/C (10% by wt) added. The mixture was evacuated and then refilled with hydrogen and stirred at room temperature for 4 h. Product filtered through Celite, washed with MeOH and concentrated under reduced pressure afforded the title compound as a cream solid, which was used in the next step without purification (16 mg, 43%). MS (ES+): m/z=377 (M+H)⁺. LC retention time: 1.5 min.

### Example 15

### 2,6-Dichloro-N-(2-[4-[methyl-(2-pyrrolidin-1-yl-ethyl)-sulfamoyl]-phenylamino]-pyrimidin-5-yl)-benzamide (IV)

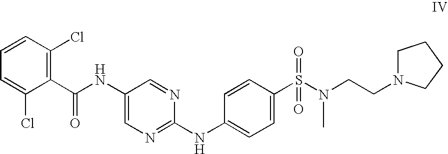

Compound 10 described in Example 14 (0.043 mmol, 1.0 equiv) and 2,6-dichlorobenzoyl chloride (0.064 mmol, 1.5 equiv) were dissolved in 8 mL of THF. TEA (0.086 mmol, 2.0 equiv) was combined via syringe and refluxed at 70° C. for 18 h under an argon atmosphere. Solvent was removed under reduced pressure and residue was suspended in EtOAc and washed with saturated NaHCO₃ and brine. The organic phase was dried (MgSO₄) and concentrated under reduced pressure. The residue was run on prep HPLC using a 10-50-75 gradient of acetonitrile and water with a flow rate of 40 mL/min to afford the TFA salt of the title compound as a yellow oil (1.25 mg, 11% yield). ¹H NMR (DMSO-d₆): δ 1.25 (s, 4H), 1.73 (t, J=7.0 Hz, 2H), 1.98 (s, 3H), 3.16 (s, 4H), 4.02 (q, J=7.2 Hz, 2H), 7.39 (t, J=7.4 Hz, 11H), 7.48 (d, J=7.6 Hz, 2H), 7.61 (t, J=7.5 Hz, 2H), 7.73 (dd, J=8.9 Hz, J=3.0 Hz, 2H), 7.99 (dd, J=22.4 Hz, J=8.9 Hz, 2H), 8.85 (d, 11H). MS (ES+): m/z=549 (M+H)⁺. LC retention time: 2.17 min.

### Example 16

### [4-(4-Methyl-piperazine-1-sulfonyl)-phenyl]-(5-nitro-pyrimidin-2-yl)-amine (11)

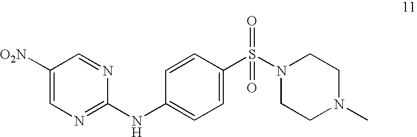

5-Nitro-pyrimidin-2-ylamine (1.78 mmol, 1.0 equiv), 1-(4-bromo-benzenesulfonyl)-4-methyl-piperazine (2.68 mmol, 1.5 equiv), Pd(OAc)₂ (0.089 mmol, 0.05eq), Xantphos (0.178 mmol, 0.1 equiv) and potassium-t-butoxide (3.56 mmol, 2.0 equiv) were suspended in 15 mL of dioxane and refluxed at 100° C. under an argon atmosphere for 18 h. The mixture was allowed to cool to room temperature, filtered and washed with DCM. The filtrate was concentrated under reduced pressure and a silica plug was run to purify material (5% MeOH/DCM) to afford the title compound as a pale yellow solid (758 mg, 95%). MS (ES+): m/z=379 (M+H)⁺. LC retention time: 1.76 min.

### Example 17

### N-[4-(4-Methyl-piperazine-1-sulfonyl)-phenyl]-pyrimidine-2,5-diamine (12)

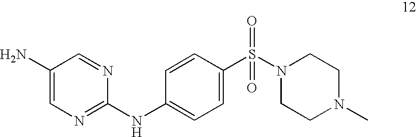

Compound 11 described in Example 16 (2.005 mmol, 1.0 equiv) dissolved in MeOH (0.05-1.0 M) was evacuated of air and placed under an argon blanket; Pd/C (10% by wt) added to reaction, evacuated and then refilled with hydrogen and stirred at room temperature for 4 h. Filtration through Celite, washed with MeOH and concentrated under reduced pressure afforded the title compound as a white solid (413 mg, 59%). The crude amino-compound was used in the next step without purification. MS (ES+): m/z=349 (M+H)⁺. LC retention time: 1.39 min.

### Example 18

### 2,6-Dichloro-N-{2-[4-(4-methyl-piperazine-1-sulfonyl)-phenylamino]-pyrimidin-5-yl}-benzamide (V)

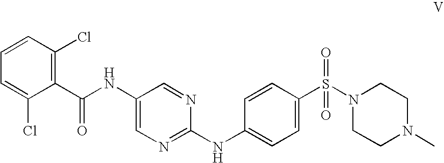

Compound 12 described in Example 17 (1.186 mmol, 1.0 equiv) and 2, 6-dichlorobenzoyl chloride (1.78 mmol, 1.5 equiv) were dissolved in 8 mL of THF. TEA (2.372 mmol, 2.0 equiv) was combined via syringe and refluxed at 70° C. for 18 h under an argon atmosphere. Solvent removed under reduced pressure, residue was suspended in EtOAc and washed with saturated NaHCO₃ and brine. The organic phase was dried (MgSO₄) and concentrated under reduced pressure. The residue was precipitated with EtOAc/DCM (1:5 v/v) to give the title compound as a cream solid (4.86 mg, 1%).

¹H NMR (DMSO-d₆): δ 2.14 (s, 3H), 2.36 (s, 4H), 2.86 (s, 4H), 7.54 (dd, J=9.3 Hz, J=7.4 Hz, 1H), 7.61 (d, J=8.7 Hz, 2H), 7.63 (d, J=9.0 Hz, 2H), 7.99 (d, J=7.0 Hz, 2H), 8.86 (s, 2H), 10.31 (s, 1H), 10.99 (s, 1H). MS (ES+): m/z=520 (M+H)⁺. LC retention time: 2.07 min.

### Example 19

### 2,6-Dimethyl-N-{2-[4-(2-pyrrolidin-1-yl-ethylsulfamoyl)-phenylamino]-pyrimidin-5-yl}-benzamide (VI)

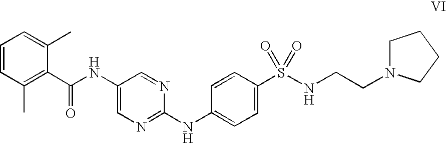

Compound 8 described in Example 11 (0.05 g, 0.14 mmol, 1 equiv) was diluted with 4 mL DCM and treated with DIEA (53 μL, 0.30 mmol, 2.2 equiv) and 2,6-dimethyl-benzoyl chloride (0.023 g, 0.14 mmol, 1 equiv). After 18 h, additional 1.0 equiv of 2,6-dimethyl-benzoyl chloride and 4 mL toluene were added. This was then heated to reflux for 2 hours. Reaction was then cooled to ambient temperature and evaporated to brown residue. HPLC purification provided the title compound as a white solid (0.01 g, 15%).

¹H NMR (DMSO-d₆): δ 1.84-1.88 (m, 2H), 1.98-2.02 (m, 2H), 2.29 (s, 6H), 2.97-3.05 (m, 4H), 3.19-3.25 (m, 2H), 3.48 (bs, 1H), 3.52-3.60 (m, 2H), 7.13 (d, J=7.6 Hz, 2H), 7.26 (t, J=7.6 Hz, 1H), 7.73 (d, J=9.0 Hz, 2H), 7.76 (t, J=6.2 Hz, 1H), 7.97 (d, J=8.9 Hz, 2H), 8.88 (s, 2H), 9.55 (bs, 1H), 10.22 (s, 1H) 10.55 (s, 1H). MS (ES+): m/z=496 (M+H)⁺. LC retention time: 2.06 min.

### Example 20

### 2-Chloro-5-methoxy-N-{2-[4-(2-pyrrolidin-1-yl-ethylsulfamoyl)-phenylamino]-pyrimidin-5-yl}-benzamide (13)

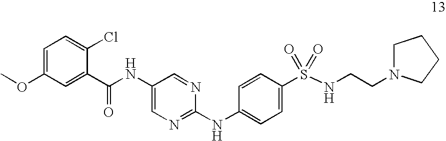

2-Chloro-5-methoxy-benzoic acid (0.051 g, 0.27 mmol, 1 equiv) was combined with 2-chloro-4,6-dimethoxy-1,3,5-triazine (CDMT) (0.058 g, 0.329 mmol, 1.2 equiv) and diluted with DCM (4 mL). This was immediately treated with 4-methyl morpholine (60 μL, 0.55 mmol, 2 equiv) and stirred at ambient temperature for 1 hour. Compound 8 described in Example 11 (0.1 g, 0.27 mmol, 1 equiv) was then added in one portion. Stirring was continued overnight. Reaction was diluted with chloroform (50 mL) and washed once with water. Aqueous phase was back extracted once with fresh chloroform. Organic phases were combined, washed once with brine and dried over sodium sulfate. Filtration followed by rotary evaporation provided crude product as yellow oil. Silica gel chromatography (6:1 DCM/MeOH) provided desired amide product as a white solid (0.065 g, 44%). MS (ES+): m/z=532 (M+H)⁺. LC retention time: 2.07 min.

### Example 21

### 2-Chloro-5-hydroxy-N-{2-[4-(2-pyrrolidin-1-yl-ethylsulfamoyl)-phenylamino]-pyrimidin-5-yl}-benzamide (VII)

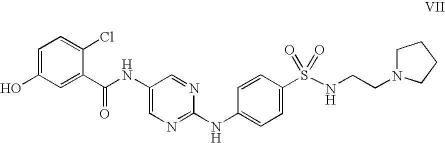

Compound 13 described in Example 20 (0.065 g, 0.12 mmol, 1 equiv) was diluted with 5 mL DCM and chilled to 0° C. using an ice bath. A 1.0 M solution of BBr₃ in DCM (1 mL, 0.99 mmol, 8 equiv) was then added in several portions resulting in dark reaction mixture. Once addition was complete, reaction was allowed to come to ambient temperature and stir for 5 hours. Reaction was then quenched by carefully pouring onto a saturated solution of sodium bicarbonate followed by sonication for 3-5 minutes. Resulting solids were filtered off. HPLC purification provided the title compound as a white solid (0.042 g, 66%).

¹H NMR (DMSO-d₆): δ 1.84-1.88 (m, 2H), 1.97-2.02 (m, 2H), 2.99-3.05 (m, 4H), 3.18-3.25 (m, 2H), 3.50-3.58 (m, 2H), 6.92 (dd, J=8.7 Hz, J=2.9 Hz, 1H), 6.96 (d, J=2.9 Hz, 1H), 7.35 (d, J=8.8 Hz, 1H), 7.73 (d, J=9.0 Hz, 2H), 7.77 (t, J=6.2 Hz, 1H), 7.98 (d, J=9.0 Hz, 2H), 8.86 (s, 2H), 9.58, (bs, 1H), 10.09 (bs, 1H), 10.22 (s, 1H), 10.62 (s, 1H). MS (ES+): m/z=519 (M+H)⁺. LC retention time: 1.85 min.

### Example 22

### 5-Bromo-pyridine-2-carboxylic acid (2-pyrrolidin-1-yl-ethyl)-amide (14)

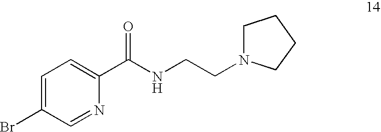

5-Bromo-pyridine-2-carboxylic acid (0.81 g, 4 mmol, 1 equiv) was combined with 2-chloro-4,6-dimethoxy-1,3,5-triazine (CDMT) (0.85 g, 4.8 mmol, 1.2 equiv) and diluted with DCM (20 mL). This was immediately treated with 4-methyl morpholine (0.81 g, 8 mmol, 2 equiv) and stirred at ambient temperature for 1 hour. 2-pyrrolidin-1-yl-ethylamine (0.46 g, 4 mmol, 1 equiv) was then added in one portion. Stirring was continued overnight. Reaction solvents were removed and residue was taken up in ethyl acetate and washed once with water. Aqueous phase was back extracted once with fresh ethyl acetate. Organic phases were combined, washed once with brine and dried over sodium sulfate. Filtration followed by rotary evaporation provided product as a yellow oil, which solidified upon standing and became yellowish solids (0.5 g, 42%).

### Example 23

### 5-(5-Nitro-pyrimidin-2-ylamino)-pyridine-2-carboxylic acid (2-pyrrolidin-1-yl-ethyl)-amide (15)

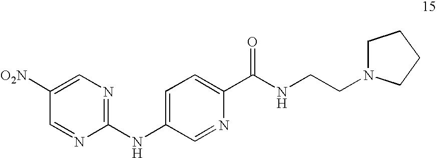

In a dry 50 mL round bottom flask, 5-nitro-pyrimidin-2-ylamine (0.2 g, 1.36 mmol, 1 equiv), compound 14 described in Example 22 (0.61 g, 2.04 mmol, 1.5 equiv), cesium carbonate (1.33 g, 4.08 mmol, 3 equiv), 4,5-bis(diphenylphosphino)-9,9-dimethyl xanthene (0.157 g, 0.272 mmol, 0.2 equiv) and tris(dibenzylideneacetone) dipalladium (0.124 g, 0.136 mmol, 0.1 equiv) were combined. Reactants were flushed with argon, diluted with dioxane (8 mL) and outfitted with reflux condenser. Reaction was heated to reflux for 18 hours. Reaction was then filtered hot and solvents were evaporated to provide dark solids. Silica gel chromatography (6:1 DCM/MeOH) provided the desired product as a yellow powder (0.17 g, 33%). R_{f}=0.23 (10% MeOH/DCM).

### Example 24

### 5-(5-Amino-pyrimidin-2-ylamino)-pyridine-2-carboxylic acid (2-Pyrrolidin-1-yl-ethyl)-amide (16)

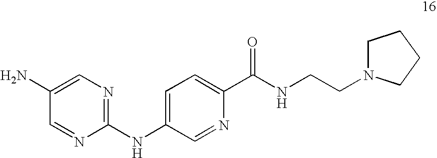

Compound 15 described in Example 23 (0.17 g, 0.476 mmol, 1 equiv) was combined with 10% palladium on carbon (0.14 g) and flushed with argon. Reactants were then diluted with methanol (15 mL) and reaction atmosphere was evacuated and replaced with hydrogen. Hydrogen balloon was affixed and reaction was allowed to stir for 2.5 hours. Argon was then bubbled through reaction mixture and contents were filtered though a pad of Celite™. Solvents were evaporated to provide crude product. Trituration with heptane followed by filtration provided the desired amine as a beige solid (0.14 g, 90%). MS (ES+): m/z=328 (M+H)⁺. LC retention time: 1.12 min.

### Example 25

### 5-[5-(2,6-Dichloro-benzoylamino)-pyrimidin-2-ylamino]-pyridine-2-carboxylic acid (2-pyrrolidin-1-yl-ethyl)-amide (VIII)

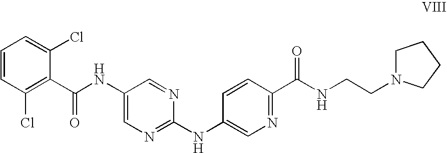

Compound 16 described in Example 24 (0.06 g, 0.183 mmol, 1.0 equiv) was dissolved in 10 mL THF and treated with 2,6-dichloro-benzoyl chloride (0.046 g, 0.22 mmol, 1.2 equiv) and stirred at ambient temperature for 5 hours. Solvents were then removed and resulting residue chromatographed. HPLC purification provided the title compound as a beige solid (0.012 g, 13%).

¹H NMR (DMSO-d₆): δ 1.85-1.89 (m, 2H), 1.97-2.03 (m, 2H), 3.01-3.09 (m, 2H), 3.31-3.38 (m, 2H), 3.59-3.67 (m, 5H), 4.20 (bs, 1H), 7.52-7.56 (m, 1H), 7.62 (d, J=8.5 Hz, 2H), 8.00 (d, J=8.7 Hz, 1H), 8.42 (dd, J=8.6 Hz, J=2.5 Hz, 1H), 8.86 (s, 2H), 8.94 (t, 6.0 Hz, 1H), 8.99 (d, J=2.6 Hz, 1H), 9.38 (bs, 1H), 10.31 (s, 1H), 11.0 (s, 1H). MS (ES+): m/z=502 (M+H)⁺. LC retention time: 1.95 min.

### Example 26

### 5-[5-(2-Chloro-5-methoxy-benzoylamino)-pyrimidin-2-ylamino]-pyridine-2-carboxylic acid (2-pyrrolidin-1-yl-ethyl)-amide (17)

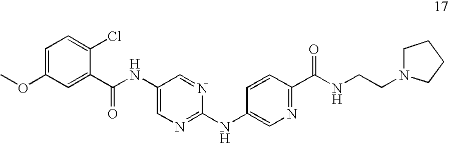

2-Chloro-5-methoxy-benzoic acid (0.046 g, 0.24 mmol, 1 equiv) was combined with 2-chloro-4,6-dimethoxy-1,3,5-triazine (CDMT) (0.052 g, 0.29 mmol, 1.2 equiv) and diluted with DCM (4 mL). This was immediately treated with 4-methyl morpholine (53 μL, 0.49 mmol, 2 equiv) and stirred at ambient temperature for 1 hour. Compound 16 described in Example 24 (0.08 g, 0.22 mmol, 1 equiv) was then added in one portion. 1 mL DMF was added to improve solubility and stirring was continued overnight. Reaction was diluted with ethyl acetate (50 mL) and washed once with water. Aqueous phase was back extracted once with fresh ethyl acetate. Organic phases were combined, washed once with brine and dried over sodium sulfate. Filtration followed by rotary evaporation provided the product as a slightly sticky white solid (0.1 g, 83%). MS (ES+): m/z=497 (M+H)⁺. LC retention time: 1.98 min.

### Example 27

### 5-[5-(2-Chloro-5-hydroxy-benzoylamino)-pyrimidin-2-ylamino]-Pyridine-2-carboxylic acid (2-pyrrolidin-1-yl-ethyl)-amide (IX)

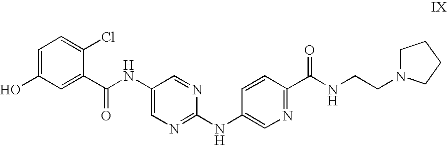

Compound 17 described in Example 26 (0.08 g, 0.1612 mmol, 1 equiv) was diluted with 10 mL DCM and chilled to 0° C. using an ice bath. A 1.0 M solution of BBr₃ in DCM (1.6 mL, 1.6 mmol, 8 equiv) was then added in several portions resulting in dark reaction mixture. Once addition was complete, reaction was allowed to come to ambient temperature and was stirred for 5 hours. Reaction was then quenched by carefully pouring onto a saturated solution of sodium bicarbonate followed by sonication for 3-5 minutes. Aqueous layer was decanted and organic phase was evaporated to brownish residue. HPLC purification provided the title compound as a white solid (0.04 g, 51%).

¹H NMR (DMSO-d₆): δ 1.84-1.87 (m, 2H), 1.99-2.02 (m, 2H), 3.00-3.09 (m, 2H), 3.33 (bs, 2H), 3.60-3.65 (m, 4H), 6.91 (dd, J=8.7 Hz, J=2.9 Hz, 1H), 6.96 (d, J=2.9 Hz, 1H), 7.35 (d, J=8.7 Hz, 1H), 8.00 (d, J=8.6 Hz, 1H), 8.43 (dd, J=8.6 Hz, J=2.5 Hz, 1H), 8.87 (s, 2H), 8.94 (t, J=6.2 Hz, 1H), 8.97 (d, J=2.4 Hz, 1H), 9.38 (bs, 1H), 10.08 (s, 1H), 10.26 (s, 1H), 10.63 (s, 1H). MS (ES+): m/z=483 (M+H)⁺. LC retention time: 1.76 min.

### Example 28

### (5-Nitropyrimidin-2-yl)-pyridin-3-yl-amine (18)

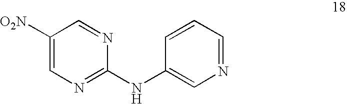

In a dry 50 mL round bottom flask 5-nitro-pyrimidin-2-ylamine (0.63 g, 4.5 mmol, 1 equiv), 3-bromo-pyridine (1.07 g, 6.8 mmol, 1.5 equiv), cesium carbonate (4.4 g, 13.5 mmol, 3 equiv), 4,5-bis(diphenylphosphino)-9,9-dimethyl xanthene (0.523 g, 9.03 mmol, 0.2 equiv) and tris(dibenzylideneacetone) dipalladium (0.42 g, 0.45 mmol, 0.1 equiv) were combined. Reactants were flushed with argon, diluted with dioxane (15 mL) and outfitted with reflux condenser. Reaction was heated to reflux for 18 hours. Reaction was then filtered hot and solvents were evaporated to provide dark solids. Silica gel chromatography (6:1 DCM/MeOH) provided the desired product as a yellow powder (0.36 g, 37%).

### Example 29

### N-Pyridin-3-yl-pyrimidine-2,5-diamine (19)

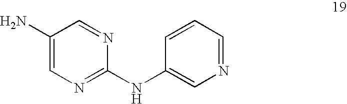

Compound 18 described in Example 28 (0.36 g, 0.476 mmol, 1 equiv) was combined with 10% palladium on carbon (0.3 g) and flushed with argon. Reactants were then diluted with methanol (15 mL) and reaction atmosphere was evacuated and replaced with hydrogen. Hydrogen balloon was affixed and reaction was allowed to stir for 2.5 hours. Argon was then bubbled through reaction mixture and contents were filtered though a pad of Celite™. Solvents were evaporated to provide crude product. Trituration with heptane followed by filtration provided the desired amine as a white solid (0.28 g, 90%).

### Example 30

### 2,6-Dichloro-N-[2-(pyridin-3-ylamino)-pyrimidin-5-yl]-benzamide (X)

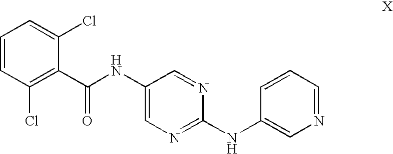

Compound 19 described in Example 29 (0.077 g, 0.41 mmol, 1.0 equiv) was dissolved in 10 mL THF, treated with 2,6-dichloro-benzoyl chloride (0.103 g, 0.494 mmol, 1.2 equiv) and stirred at ambient temperature for 4 hours. Solvents were then removed and resulting residue chromatographed to afford the title compound as a beige solid (0.026 g, 18%).

¹H NMR (DMSO-d₆): δ 3.73 (bs, 1H), 7.54-7.57 (m, 1H), 7.62 (d, J=8.7 Hz, 2H), 7.73-7.76 (m, 1H), 8.37 (bs, 1H), 8.49 (d, J=8.7 Hz, 1H), 8.88 (s, 2H), 9.20 (bs, 1H), 10.45 (s, 1H), 11.03 (s, 1H). MS (ES+): m/z=360 (M+H)⁺. LC retention time: 1.84 min.

### Example 31

### 2-Chloro-5-methoxy-N-[2-(pyridin-3-ylamino)-pyrimidin-5-yl]-benzamide (20)

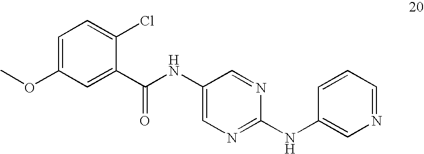

2-Chloro-5-methoxy-benzoic acid (0.073 g, 0.392 mmol, 1 equiv) was combined with 2-chloro-4,6-dimethoxy-1,3,5-triazine (CDMT) (0.0828 g, 0.47 mmol, 1.2 equiv) and diluted with DCM (10 mL). This was immediately treated with 4-methyl morpholine (0.086 mL, 0.785 mmol, 2 equiv) and allowed to stir at ambient temperature for 1 hour. Compound 19 described in Example 29 (0.073 g, 0.392 mmol, 1 equiv) was then added in one portion. After 2 h, 1 mL DMF was added to improve solubility. Stirring was continued overnight. Reaction solvents were removed and residue was taken up in DCM and loaded onto silica gel column. Chromatography (100% EtOAc) provided the desired product as a white powder (0.13 g, 95%).

### Example 32

### Chloro-5-hydroxy-N-[2-(pyridin-3-ylamino)-pyrimidin-5-yl]-benzamide

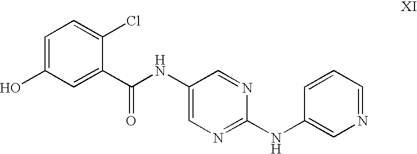

Compound 20 described in Example 31 (0.092 g, 0.26 mmol, 1 equiv) was diluted with 10 mL DCM and chilled to 0° C. using an ice bath. A 1.0 M solution of BBr₃ in DCM (2.0 mL, 2.07 mmol, 8 equiv) was then added in several portions resulting in dark reaction mixture. Once addition was complete, reaction was allowed to come to ambient temperature and was stirred for 5 hours. Reaction was then quenched by carefully pouring onto a saturated solution of sodium bicarbonate followed by sonication for 3-5 minutes. Aqueous layer was decanted and organic phase was evaporated to brownish residue. HPLC purification provided the title compound as a white solid (0.03 g, 34%).

¹H NMR (DMSO-d₆): δ 3.80 (bs, 1H), 6.91 (dd, J=2.9 Hz, J=8.7 Hz, 1H), 6.97 (d, J=2.9 Hz, 1H), 7.35 (d, J=8.6 Hz, 1H), 7.73-7.77 (m, 1H), 8.36 (bs, 1H), 8.49 (d, J=8.6 Hz, 1H), 8.89 (s, 2H), 9.20 (bs, 1H), 10.09 (bs, 1H), 10.41 (s, 1H), 10.67 (s, 1H). MS (ES+): m/z=343 (M+H)⁺. Retention time: 1.64 min.

### Example 33

### (5-Nitro-pyrimidin-2-yl)-[4-(2-pyrrolidin-1-yl-ethoxy)-phenyl]-amine (21)

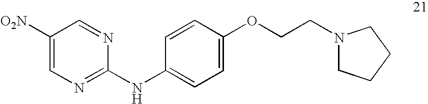

In a dry 100 mL round bottom flask 5-nitro-pyrimidin-2-ylamine (2 g, 14.3 mmol, 1 equiv), 1-[2-(4-bromo-phenoxy)-ethyl]-pyrrolidine (4.45 mL, 21.4 mmol, 1.5 equiv), cesium carbonate (14 g, 42.9 mmol, 3 equiv), 4,5-bis(diphenylphosphino)-9,9-dimethyl xanthene (1.65 g, 1.43 mmol, 0.2 equiv) and tris(dibenzylideneacetone) dipalladium (1.3 g, 0.714 mmol, 0.1 equiv) were combined. Reactants were flushed with argon, diluted with dioxane (50 mL) and outfitted with reflux condenser. Reaction was heated to reflux for 18 hours. Reaction was cooled to room temperature and filtered. Silica gel chromatography provided the desired nitro product as a yellow powder (1.5 g, 32%).

### Example 34

### N-[4-(2-Pyrrolidin-1-yl-ethoxy)-phenyl]-pyrimidine-2,5-diamine (22)

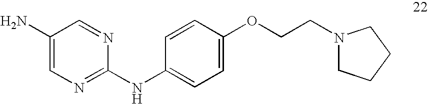

A methanolic solution of compound 21 described in Example 33 (1.5 g, 6.48 mmol) was purged with argon for several minutes then treated with 10% Palladium on carbon (0.85 g). Reaction atmosphere was evacuated and replaced with hydrogen added via hydrogen-filled balloon. After 2 hours, hydrogen balloon removed and reaction solvents purged with argon. Celite was added to reaction solvent and resulting slurry was filtered through pad of celite. Solvents were then removed providing the desired amine as a yellow solid (0.36 g, 26%).

### Example 35

### N′-(2,6-Dichloro-benzyl)-N-[4-(2-pyrrolidin-1-yl-ethoxy)-phenyl]-pyrimidine-2,5-diamine (XII)

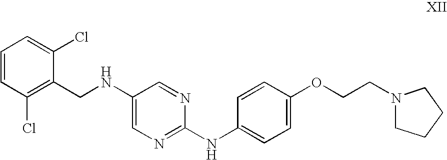

2-Bromomethyl-1,3-dichloro-benzene (0.45 g, 1.87 mmol, 1.4 equiv) was combined with compound 22 described in Example 34 (0.4 g, 1.34 mmol, 1 equiv), cesium carbonate (1.09 g, 3.34 mmol, 2.5 equiv) and diluted with dioxane (25 mL). This was heated to 100° C. and stirred overnight. Reaction solvents were then removed and resulting crude solids were purified via silica gel chromatography. Product isolated as yellow oil (0.20 g) which was then diluted with DCM (10 mL) and treated with 0.33 mL 4M HCl/Ether. Solvents then removed yielding HCl salt of the desired product as a pale yellow solid (0.20 g, 33%).

¹H NMR (DMSO-d₆): δ 1.85-1.9 (m, 2H), 1.96-2.05 (m, 2H), 3.05-3.1 (m, 2H), 3.55-3.61 (m, 8H), 4.27 (t, J=4.8 Hz, 2H), 4.39 (s, 2H), 6.9 (d, J=9.15 Hz, 2H), 7.38 (t, J=7.65 Hz, 1H), 7.52 (d, J=8.05 Hz, 2H), 7.6 (d, J=9.1 Hz, 2H), 8.08 (s, 2H), 8.97 (bs, 1H), 10.61 (bs, 1H).

### Example 36

### 3-(3-Bromo-phenyl)-propan-1-ol (23)

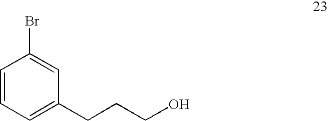

3-(3-Bromo-phenyl)-propionic acid (3.88 g, 16.9 mmol, 1 equiv) was diluted with THF (50 mL) and chilled to 0° C. 1M LAH solution added slowly so as to not allow internal reaction temperature to climb above 10-15° C. Once LAH addition was complete, reaction allowed to come to room temperature and stir for 3 h. Reaction then quenched with sequential addition of water (0.5 mL), 15% NaOH (0.5 mL) and water again (1.5 mL). This was then filtered and solvents evaporated to provide the product as a pale oil (2.75 g, 98%). R_{f}=0.42 (30% EtOAc/hexanes).

### Example 37

### 1-Bromo-3-(3-bromo-propyl)-benzene (24)

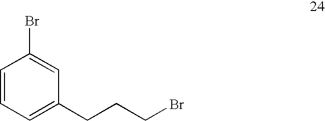

Alcohol 23 described in Example 36 (4 g, 18.6 mmol, 1 equiv) was diluted with THF (100 mL) and treated with CBr₄ (9.27 g, 27.9 mmol, 1.5 equiv), triphenyl phosphine (7.31 g, 27.9 mmol, 1.5 equiv). and subsequently stirred for 16 h. Reaction was then diluted with EtOAc (125 mL) and washed with brine (2×75 mL). Organic phase was cut from aqueous phase, dried over sodium sulfate, filtered and evaporated to provide the desired bromide as clear oil (4 g, 78%).

### Example 38

### 1-[3-(3-Bromo-phenyl)-propyl]-pyrrolidine (25)

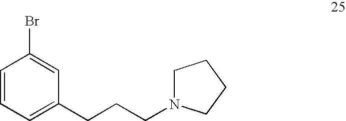

Bromide 24 described in Example 37 (1 g, 3.68 mmol, 1 equiv) was diluted with dioxane (30 mL), treated with pyrrolidine (0.61 mL, 7.35 mmol, 2 equiv), cesium carbonate (2.4 g, 7.35 mmol, 2 equiv) and stirred for 18 h. Reaction was then diluted with water (125 mL) and extracted with EtOAc (2×100 mL). Organic phase was cut from aqueous phase, dried over sodium sulfate, filtered and evaporated to provide the desired product as clear oil (0.6 g, 61%).

### Example 39

### (5-Nitro-pyrimidin-2-yl)-[3-(3-pyrrolidin-1-yl-propyl)-phenyl]-amine (26)

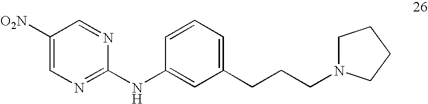

In a dry 50 mL round bottom flask 5-nitro-pyrimidin-2-ylamine (0.35 g, 2.5 mmol, 1 equiv), compound 25 described in Example 38 (0.8 g, 3 mmol, 1.2 equiv), cesium carbonate (2.43 g, 7.5 mmol, 3 equiv), 4,5-bis(diphenylphosphino)-9,9-dimethyl xanthene (0.288 g, 0.5 mmol, 0.2 equiv) and tris(dibenzylideneacetone) dipalladium (0.228 g, 0.25 mmol, 0.1 equiv) were combined. Reactants were flushed with argon, diluted with dioxane (20 mL) and outfitted with reflux condenser. Reaction was heated to reflux for 18 hours. Reaction was then cooled to room temperature and filtered. Silica gel chromatography provided the desired nitro product as a yellow powder (0.5 g, 61%).

### Example 40

### N-[3-(3-Pyrrolidin-1-yl-propyl)-phenyl]-pyrimidine-2,5-diamine (27)

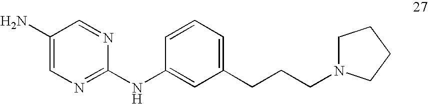

Compound 26 described in Example 39 (0.15 g, 0.459 mmol, 1 equiv) was combined with 10% palladium on carbon (0.10 g) and flushed with argon. Reactants were then diluted with methanol (25 mL) and reaction atmosphere was evacuated and replaced with hydrogen. Hydrogen balloon was affixed and reaction was allowed to stir for 2.5 hours. Argon was then bubbled through reaction mixture and contents were filtered though a pad of Celite™. Solvents were evaporated to provide crude product. Trituration with heptane followed by filtration provided the desired amine as a yellow solid (0.10 g, 74%).

### Example 41

### 2,6-Dichloro-N-{2-[3-(3-pyrrolidin-1-yl-propyl)-phenylamino]-Pyrimidin-5-yl}-benzamide (XIII)

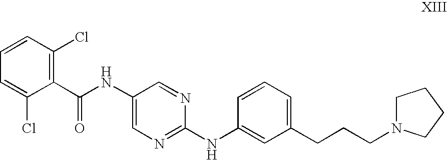

Amine 27 described in Example 40 (0.138 g, 0.47 mmol, 1 equiv) was diluted with THF (15 mL), treated with 2,6-dichloro-benzoyl chloride (0.116 mL, 0.56 mmol, 1.2 equiv) and stirred for 18 h. Reaction solvents were then removed and resulting crude solids were purified via HPLC to afford the title compound as a white solid (0.140 g, 64%).

¹H NMR (DMSO-d₆): δ 1.8-1.88 (m, 2H), 1.9-2.05 (m, 4H), 2.61 (t, J=7.65 Hz, 2H), 2.95-3.04 (m, 2H), 3.1-3.18 (m, 2H), 3.5-3.59 (m, 2H), 6.81-6.82 (d, J=7.5 Hz, 1H), 7.22 (t, J=7.85 Hz, 1H), 7.5-7.53 (m, 1H), 7.58-7.62 (m, 4H), 8.75 (s, 2H), 9.59 (bs, 1H), 9.69 (s, 1H), 10.87 (s, 1H).

### Example 42

### (4-(5-Nitropyrimidin-2-ylamino)phenyl)(4-methylpiperazin-1-yl)methanone (28)

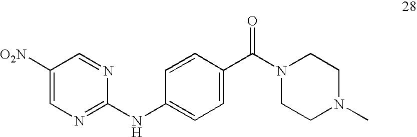

A mixture of 5-nitro-pyrimidin-2-ylamine (504 mg, 3.6 mmol), (4-bromo-phenyl)-(4-methyl-piperazin-1-yl)-methanone (1.1 g, 3.9 mmol), Cs₂CO₃ (4.6 g, 14.2 mmol), Xantphos (420 mg, 0.7 mmol), Pd₂(dba)₃ (330 mg, 0.4 mmol), and 3 Å mol sieves in dioxane (70 mL) was purged with argon for 5 min, and was heated to reflux for 18 h. under argon. Dioxane was removed in vacuo and the resulting mixture was partitioned between EtOAc and water (200 mL each). The layers were separated and the aqueous layer was extracted twice more with EtOAc (200 mL). The organic layers were combined and concentrated in vacuo. The crude product was purified by flash column chromatography (0.5% NH₄OH/10% MeOH/89.5% dichloromethane) to afford an off-white solid (657 mg, 53%).

R_{f} 0.07 (0.5% NH₄OH, 10% MeOH in CHCl₃). ¹H NMR (DMSO-d₆) δ 2.19 (s, 3H), 2.31 (bs, 4H), 3.48 (bs, 4H), 7.38 (d, J=8.6 Hz, 2H), 7.69 (d, J=8.4 Hz, 2H), 9.16 (s, 2H), 11.01 (bs, 1H). MS (ES+): m/z=343 (M+H)⁺. LC retention time: 1.62 min.

### Example 43

### (4-(5-Aminopynrimidin-2-ylamino)phenyl)(4-methylpiperazin-1-yl)methanone (29)

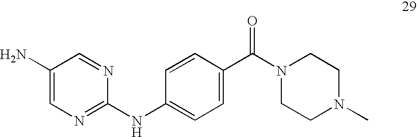

To intermediate 28 described in Example 42 (656 mg, 1.9 mmol) in THF (30 mL), was added 10% Pd/C (655 mg, 1.9 mmol) after flushing with argon. The suspension was bubbled with hydrogen for 10 min. and was stirred under H₂ atmosphere for 2 h. The suspension was purged with argon and filtered through celite using methanol to thoroughly wash the filter cake. The organic solution was concentrated in vacuo and taken up in toluene and concentrated again in vacuo to afford an off-white solid (608 mg, quant). MS (ES+): m/z=313 (M+H)⁺. LC retention time: 0.72 min

### Example 44

### 2-(5-Nitropyrimidin-2-ylamino)-N-(2-(pyrrolidin-1-yl)ethyl)thiazole-4-carboxamide (30)

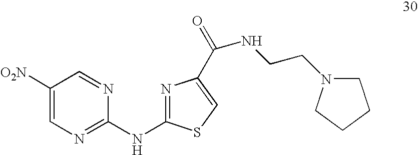

A mixture of 5-nitro-pyrimidin-2-ylamine (251 mg, 1.8 mmol), 2-bromo-thiazole-4-carboxylic acid (2-pyrrolidin-1-yl-ethyl)-amide (540 mg, 1.8 mmol), Cs₂CO₃ (2.3 g, 7.1 mmol), Xantphos (211 mg, 0.4 mmol), and Pd₂(dba)₃ (161 mg, 1.2 mmol) in dioxane (36 mL) was purged with argon for 5 min. The reaction slurry was heated to reflux for 18 h. under argon. Dioxane was removed in vacuo and the resulting crude mixture was adsorbed on silica gel and purified using an Isco flash chromatography system (0% to 30% Methanol with 1% NH₄OH in DCM) to afford a white solid (270 mg, 42%). R_{f} 0.31 (0.5% NH₄OH, 10% MeOH in CHCl₃). MS (ES+): m/z=364 (M+H)⁺. LC retention time: 1.74 min

### Example 45

### 2-(5-Aminopyrimidin-2-ylamino)-N-(2-(pyrrolidin-1-yl)ethyl)thiazole-4-carboxamide (31)

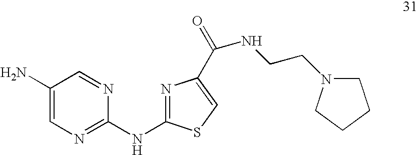

To intermediate 30 described in Example 44 (270 mg, 0.7 mmol) in MeOH (3 mL) and THF (60 mL) was added 10% Pd/C (270 mg, 0.07 mmol) after flushing with argon. The suspension was bubbled with hydrogen for 10 min. and was stirred under H₂ atmosphere for 2 h. The suspension was purged with argon and filtered through celite using methanol to thoroughly wash the filter cake. The organic solution was concentrated in vacuo and taken up MeOH and DCM. The product was precipitated with ether and hexanes to afford an off-white solid (crude: 192 mg, 79%; after recrystallization: 136.7 mg, 56%).

¹H NMR (DMSO-d₆) δ 1.71 (bs, 4H), 2.53 (bs, 4H), 2.61 (bs, 2H), 3.38-3.41 (m, 2H), 5.09 (s, 2H), 7.52 (s, 1H), 7.70 (bs, 1H) 8.06 (s, 2H), 11.22 (bs, 1H). MS (ES+): m/z=334 (M+H)⁺. LC retention time: 1.30 min.

### Example 46

### 3-Bromo-N-(2-hydroxyethyl)-N-isopropylbenzamide (32)

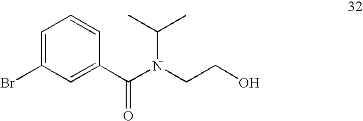

To a mixture of 2-isopropylamino-ethanol (1.5 mL, 9.1 mmol, 70% pure) and TEA (2.5 mL, 18 mmol) in DCM (40 mL) was added 3-bromo-benzoyl chloride (1 mL, 7.6 mmol) in a single portion. The reaction mixture was stirred for 30 min and was successively washed with 10% NaHCO₃ (20 mL) and brine (20 mL), dried (Na₂SO₄), and concentrated in vacuo. Recrystallization in Acetone/EtOAc/Hexanes mixture afforded the title compound as a white solid (1.78 g, 82%). R_{f} 0.33 (EtOAc). MS (ES+): m/z=286/288 (M+H)⁺. LC retention time: 2.32 min.

### Example 47

### 3-(5-Nitropyrimidin-2-ylamino)-N-(2-hydroxyethyl)-N-isopropylbenzamide (33)

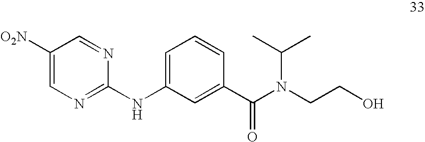

A mixture of 5-nitro-pyrimidin-2-ylamine (141 mg, 1.0 mmol), bromide intermediate 32 described in Example 46 (301 mg, 1.1 mmol), Cs₂CO₃ (1.3 g, 4.0 mmol), Xantphos (117 mg, 0.2 mmol), and Pd₂(dba)₃ (92 mg, 0.1 mmol) in dioxane (20 mL) was purged with argon for 5 min, and the suspension was heated to reflux for 16 h. under argon. Dioxane was removed in vacuo, and the crude mixture was adsorbed onto silica gel and purified using an Isco flash chromatography system (0% to 30% Methanol with 1% NH₄OH in DCM) to afford a tan solid (142 mg, 41%).

¹H NMR (DMSO-d₆) δ 1.11 (bs, 6H), 3.33-3.36 (m, 2H), 3.56 (bs, 2H), 3.87 (bs, 1H), 4.76 (bs, 1H), 7.08 (d, J=7.6 Hz, 1H), 7.43 (t, J=7.9 Hz, 1H), 7.78 (bs, 1H), 7.79 (dd, J=8.0, 1.5 Hz, 1H), 9.25 (s, 2H), 10.94 (s, 1H). MS (ES+): m/z=346 (M+H)⁺. LC retention time: 2.21 min.

### Example 48

### 3-(5-Aminopyrimidin-2-ylamino)-N-(2-hydroxyethyl)-N-isopropylbenzamide (34)

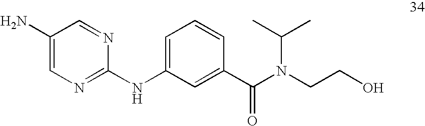

To intermediate 33 described in Example 47 (140 mg, 0.4 mmol) in 1:1 THF/MeOH (10 mL) was added 10% Pd/C (143 mg, 0.04 mmol) after flushing with argon, and the resulting suspension was bubbled with hydrogen for 10 min. The reaction was then allowed to stir under H₂ atmosphere for 90 min. The suspension was purged with argon and filtered through celite using methanol to thoroughly wash the filter cake. The organic solution was concentrated in vacuo to afford a tan solid (90 mg, 71%). MS (ES+): m/z=316 (M+H)⁺. LC retention time: 1.55 min.

### Example 49

### 4-Bromobenzoyl chloride (35)

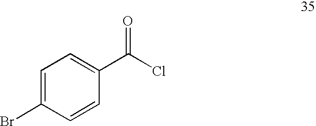

To acid 4-bromo-benzoic acid (5 g, 24.9 mmol) in DCM (40 mL) was added oxalyl chloride (3.4 mL, 39.6 mmol) followed by DMF (0.25 mL, 3.2 mmol). On addition of DMF, vigorous bubbling ensued. Bubbling stopped after ca. 45 min. The reaction was stirred for 15 min more for a total of 1 hour. The reaction mixture was carefully concentrated in vacuo to afford a crude yellow-brown solid which was used as is without further purification (5.6 g, quant).

### Example 50

### 4-Bromo-N-(2-hydroxyethyl)-N-isopropylbenzamide (36)

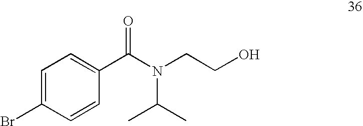

To a mixture of acid chloride intermediate 35 described in Example 49 (3.26 g, 14.9 mmol), and TEA (9.2 mL, 66.2 mmol) in DCM (140 mL), was added 2-isopropylamino-ethanol (2.5 mL, 15.2 mmol) in a single portion and was to stirred for 30 min. The reaction was concentrated in vacuo, adsorbed onto silica gel and purified using an Isco flash chromatography system (100% EtOAC) to afford a white solid (2.62 g, 62%).

R_{f} 0.29(0.5% NH₄OH, 10% MeOH in CHCl₃). ¹H NMR (DMSO-d₆) δ 1.07 (bs, 6H), 3.30 (bs, 2H), 3.54 (bs, 2H), 3.73 (bs, 1H), 4.74 (bs, 1H), 7.30 (dt, J=8.7, 2.1 Hz, 2H), 7.63 (bd, J=8.0 Hz, 2H). MS (ES+): m/z=286/288 (M+H)⁺. LC retention time: 2.35 min.

### Example 51

### 4-(5-Nitropyrimidin-2-ylamino)-N-(2-hydroxyethyl)-N-isopropylbenzamide (37)

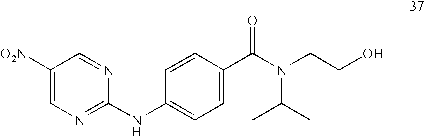

A mixture of 5-nitro-pyrimidin-2-ylamine (142 mg, 1.0 mmol), bromide intermediate 36 described in Example 50 (285 mg, 1.0 mmol), Cs₂CO₃ (1.4 g, 4.3 mmol), Xantphos (114 mg, 0.2 mmol), and Pd₂(dba)₃ (91 mg, 0.1 mmol) in dioxane (20 mL) was purged with argon for 5 min, and the ssuspension was heated to reflux for 2.5 hours under argon. Dioxane was removed in vacuo, and the crude mixture was adsorbed onto silica gel and purified using an Isco flash chromatography system (0% to 10% Methanol with 1% NH₄OH in DCM) to afford a crude tan solid (357 mg, quant.).

¹H NMR (DMSO-d₆) δ 1.10 (bs, 6H), 3.54 (bs, 2H), 4.74 (bs, 1H), 7.36 (d, J=8.5 Hz, 2H), 7.82 (d, J=8.5 Hz, 2H), 9.26 (s, 2H), 10.99 (s, 1H). MS (ES+): m/z=346 (M+H)⁺. LC retention time: 2.18 min.

### Example 52

### 4-(5-Aminopyrimidin-2-ylamino)-N-(2-hydroxyethyl)-N-isopropylbenzamide (38)

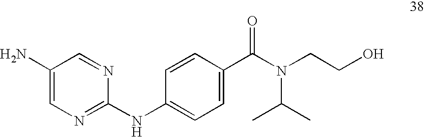

To intermediate 37 described in Example 51 (357 mg, 1.0 mmol) in 1:1 THF/MeOH (26 mL) was added 10% Pd/C (360 mg, 0.01 mmol) after flushing with argon, and the resulting suspension was bubbled with hydrogen for 10 min. The reaction was then allowed to stir under H₂ atmosphere for 18 h. The suspension was purged with argon and filtered through celite using methanol to thoroughly wash the filter cake. The organic solution was concentrated in vacuo and triturated with ether to afford a tan solid that was collected by filtration (252 mg, 78%). MS (ES+): m/z=316 (M+H)⁺. LC retention time: 1.51 min.

### Example 53

### 2-Chloro-5-methoxybenzoic acid (39)

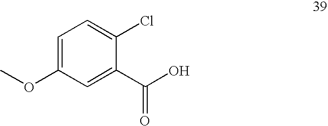

A solution of 2-chloro-5-methoxy-bromobenzene (5 g, 22.6 mmol) in THF (55 mL) was cooled to −78° C. and 2.5 M nBuLi in hexanes (10.8 mL) was added dropwise over 15 min keeping the temperature of the reaction below −60° C. When the reaction had cooled fully back to −78° C., 13 pieces of solid CO₂ (2-4 cm by 1 cm cylinders) were rubbed free of ice and added slowly to the reaction. The cold bath was removed and the reaction allowed to slowly warm to room temperature (ca. 1.5 hours). The reaction was diluted with 85 mL EtOAc and 100 mL NaHCO₃ (sat.). The pH was adjusted to 10-12 with 30% NaOH. The layers were separated and the aqueous layer was acidified with HCl (conc.) to precipitate the title compound as an off-white solid which was collected by filtration and rinsed with cold water. Traces of solvents were removed in vacuo (2.2 g, 52%).

¹H NMR (DMSO-d₆) δ 3.33 (s, 3H), 7.10 (dd, J=8.9, 3.3 Hz, 1H), 7.28 (d, J=3.2 Hz, 1H), 7.43 (d, J=8.9 Hz, 1H). MS (ES+): m/z=169 (M+H)⁺. LC retention time: 2.20 min.

### Example 54

### 2,6-Dichloro-N-{2-[4-(4-methyl-piperazine-1-carbonyl)-phenylamino]-pyrimidin-5-yl}-benzamide (XIV)

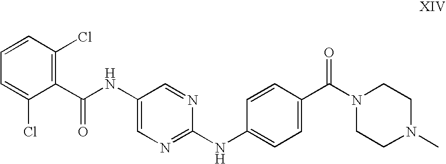

To intermediate 29 described in Example 43 (50 mg, 0.2 mmol) in THF (25 mL) was added 2,6-dichlorobenzoyl chloride (1.5 mL, 10.7 mmol) in a single portion, and the reaction was stirred for 15 min. at which point a solid had formed. The reaction was stirred for 18 h. and the crude mixture was purified by HPLC to afford the title compound as a yellow solid (14 mg, 15%).

¹H NMR (DMSO-d₆) δ 2.83 (s, 314), 3.08 (bs, 2H), 3.27 (bs, 2H), 4.23 (bs, 2H), 7.42 (d, J=8.8 Hz, 2H), 7.54 (dd, J=9.0, 7.1 Hz, 1H), 7.62 (d, J=8.8 Hz, 2H), 7.86 (d, J=8.7 Hz, 2H), 8.81 (s, 2H), 9.86 (bs, 1H), 10.05 (s, 1H), 10.94 (s, 1H). MS (ES+): m/z=485/487/489 (M+H)⁺. LC retention time: 1.89 min.

### Example 55

### 2,6-Dimethyl-N-{2-[4-(4-methyl-piperazine-1-carbonyl)-phenylamino]-pyrimidin-5-yl}-benzamide (XV)

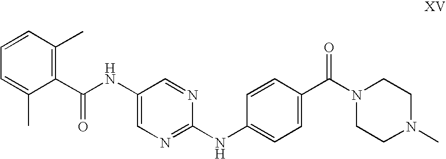

2,6-Dimethylbenzoic acid (29 mg, 0.19 mmol) was converted to the corresponding acid chloride employing a procedure similar to that for intermediate 35 described in Example 49, using DCM (1 mL), oxalyl chloride (0.026 mL, 0.3 mmol), and DMF (ca. 20 μL). When the reaction was complete (ca. 20 min.), the solution was carefully concentrated in vacuo. THF (1 mL) was added to dissolve the acid chloride followed by amine intermediate 29 described in Example 43 (52 mg, 0.17 mmol). The reaction formed a precipitate over 18 h. and was purified by HPLC to afford a pale-yellow solid (36 mg, 40%).

¹H NMR (DMSO-d₆) δ 2.30 (s, 6H), 2.83 (s, 3H), 3.08 (bs, 2H), 3.27 (bs, 2H), 3.42-3.48 (m, 2H), 4.22 (bs, 2H), 7.13 (d, J=7.6 Hz, 2H), 7.26 (t, J=7.6 Hz, 1H), 7.42 (d, J=8.8 Hz, 2H), 7.86 (dd, J=6.9, 1.9 Hz, 2H), 8.84 (s, 2H), 9.85 (bs, 1H), 9.99 (s, 1H), 10.50 (s, 1H). MS (ES+): m/z=446 (M+H)⁺. LC retention time: 1.86 min.

### Example 56

### 2-Chloro-5-methoxy-N-{2-[4-(4-methyl-piperazine-1-carbonyl)-phenylamino]-pyrimidin-5-yl}-benzamide (40)

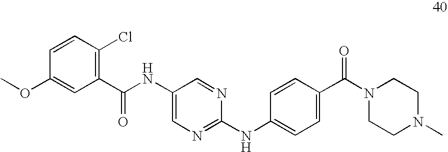

Acid intermediate 39 described in Example 53 (74 mg, 0.4 mmol) was converted to the corresponding acid chloride employing a procedure similar to that for intermediate 35 described in Example 49, using DCM (3 mL), oxalyl chloride (0.053 mL, 0.6 mmol), and DMF (ca. 0.02 mL). When the reaction was complete (ca. 45 min.), the solution was carefully concentrated in vacuo. THF (3 mL) was added to dissolve the acid chloride followed by amine intermediate 29 described in Example 43 (104 mg, 0.3 mmol). The reaction formed an immediate precipitate and was allowed to stir for 18 h. The solid was collected by vacuum filtration, rinsed with ether and residual solvent was removed in vacuo to afford a white solid (117 mg, 68%). MS (ES+): m/z=481/483 (M+H)⁺. LC retention time: 1.89 min.

### Example 57

### 2-Chloro-5-hydroxy-N-{2-[4-(4-methyl-piperazine-1-carbonyl)-phenylamino]-pyrimidin-5-yl}-benzamide (XVI)

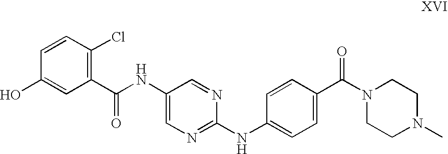

To a suspension of the HCl salt of compound 40 described in Example 56 (117 mg, 0.2 mmol) in DCM (20 mL) was added 1.5 mL of 1M BBr₃ in DCM (1.5 mmol). After 1 hour, an additional 0.138 mL of neat BBr₃ (1.5 mmol) was added. The reaction was stirred for 18 h., and an additional 0.138 mL of neat BBr₃ (1.5 mmol) was added and stirred for another 24 h. The reaction was quenched with NaHCO₃ and concentrated in vacuo. The crude residue was purified by HPLC to afford a pale-yellow solid (13 mg, 10%).

¹H NMR (DMSO-d₆) δ 2.83 (s, 3H), 3.05-3.12 (m, 2H), 3.26 (bs, 2H), 3.41-3.47 (m, 2H), 4.15 (bs, 2H), 6.91 (dd, J=8.8, 2.9 Hz, 1H), 6.96 (d, J=2.9 Hz, 1H), 7.35 (d, J=8.8 Hz, 1H), 7.42 (d, J=8.7 Hz, 2H), 7.86 (d, J=8.8 Hz, 2H), 8.82 (s, 2H), 9.78 (bs, 1H), 9.99 (s, 1H), 10.07 (bs, 1H), 10.57 (s, 1H). MS (ES+): m/z=467/469 (M+H)⁺. LC retention time: 1.68 min.

### Example 58

### 2-Methyl-3-acetoxy-N-{2-[4-(4-methyl-piperazine-1-carbonyl)-phenylamino]-pyrimidin-5-yl}-benzamide (41)

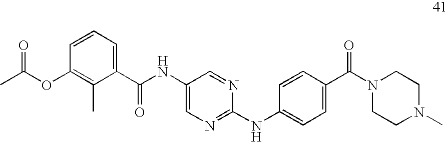

The title compound was synthesized employing a procedure similar to that described for compound XIV described in Example 54 using 3-(chlorocarbonyl)-2-methylphenyl acetate (75 mg, 0.35 mmol) and amine intermediate 29 described in Example 43 (104 mg, 0.33 mmol) in THF (3 mL). The product was isolated to afford a white solid (131 mg, 75%). MS (ES+): m/z=490 (M+H)⁺. LC retention time: 1.80 min.

### Example 59

### 2-Methyl-3-hydroxy-N-{2-[4-(4-methyl-piperazine-1-carbonyl)-phenylamino]-pyrimidin-5-yl}-benzamide (XVII)

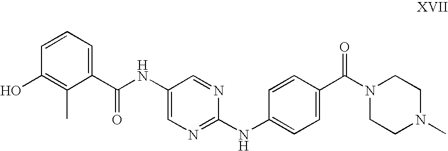

To a suspension of the HCl salt of compound 41 described in Example 58 (131 mg, 0.27 mmol) in methanol (1 mL) was added 0.5 M sodium methoxide in methanol (1 mL, 0.5 mmol). An immediate precipitate formed, and after 10 min., HCl was added to quench the reaction. The mixture was purified by HPLC to remove excess salts and the title compound was isolated as a yellow solid (88 mg, 58%).

¹H NMR (DMSO-d₆) δ 2.18 (s, 3H), 2.83 (s, 3H), 3.03-3.13 (m, 2H), 3.27 (bs, 2H), 3.40-3.49 (m, 2H), 4.24 (bs, 2H), 6.92 (d, J=7.4 Hz, 1H), 6.93 (d, J=8.1 Hz, 1H), 7.12 (t, J=7.8 Hz, 1H), 7.42 (d, J=8.7 Hz, 2H), 7.86 (d, J=8.8 Hz, 2H), 8.84 (s, 2H), 9.65 (bs, 1H), 9.80 (bs, 1H), 9.95 (s, 1H), 10.34 (s, 1H). MS (ES+): m/z=447 (M+H)⁺. LC retention time: 1.56 min.

### Example 60

### 2-[5-(2-Chloro-5-methoxy-benzoylamino)-pyrimidin-2-ylamino]-thiazole-4-carboxylic acid (2-pyrrolidin-1-yl-ethyl)-amide (42)

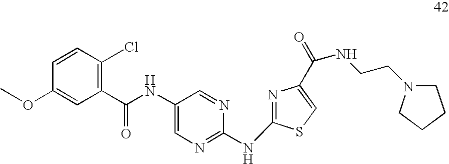

Employing a procedure similar to that used for compound 40 described in Example 56 with acid intermediate 39 described in Example 53 (74 mg, 0.40 mmol), oxalyl chloride (0.053 mL, 0.62 mmol), and amine 31 described in Example 45 (115 mg, 0.35 mmol), the title compound was isolated after HPLC purification, as a yellow solid (89 mg, 41%). MS (ES+): m/z=502 (M+H)⁺. LC retention time: 2.01 min.

### Example 61

### 2-[5-(2-Chloro-5-hydroxy-benzoylamino)-pyrimidin-2-ylamino]-thiazole-4-carboxylic acid (2-pyrrolidin-1-yl-ethyl)-amide (XVIII)

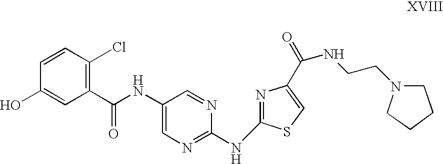

Employing a procedure similar to that described for compound XVI described in Example 57, using the HCl salt of compound 42 described in Example 60 (89 mg, 0.17 mmol), and a single addition of BBr₃ (0.156 mL, 1.7 mmol) in 15 mL DCM over 150 min. afforded the TFA salt of the title compound as a white solid (19.1 mg, 19%).

¹H NMR (DMSO-d₆) δ 1.82-1.92 (m, 2H), 1.97-2.06 (m, 2H), 3.01-3.09 (m, 2H), 3.33 (q, J=5.8 Hz, 2H), 3.60-3.64 (m, 2H), 6.92 (dd, J=8.8, 2.9 Hz, 1H), 6.98 (d, J=2.9 Hz, 1H), 7.36 (d, J=8.8 Hz, 1H), 7.75 (s, 1H), 8.15 (t, J=6.1 Hz, 1H), 8.95 (s, 2H), 9.40 (bs, 1H), 10.09 (s, 1H), 10.71 (s, 1H), 11.83 (s, 1H). MS (ES+): m/z=488 (M+H)⁺. LC retention time: 1.80 min.

### Example 62

### 2,6-Dichloro-N-(2-{3-[(2-hydroxy-ethyl)-isopropyl-carbamoyl]-phenylamino}-pyrimidin-5-yl)-benzamide (XIX)

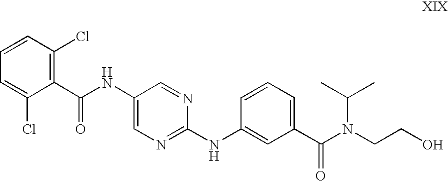

Employing a procedure similar to that described for compound XIV described in Example 54, using amine intermediate 34 described in Example 48 (45 mg, 0.14 mmol), 2,6-dichlorobenzoylchloride (0.022 mL, 0.16 mmol), and TEA (0.040 mL, 0.28 mmol) in THF (2 mL) afforded the title compound as a pale-yellow solid (45 mg, 64%).

¹H NMR (DMSO-d₆) δ 1.11 (bs, 6H), 3.29-3.37 (m, 2H), 3.53-3.54 (m, 2H), 3.88-3.95 (m, 2H), 6.89 (d, J=7.4 Hz, 1H), 7.33 (t, J=7.9 Hz, 1H), 7.53 (dd, J=9.0, 7.2 Hz, 1H), 7.58-7.63 (m, 2H), 7.74-7.78 (m, 2H), 8.77 (s, 2H), 10.89 (s, 1H). MS (ES+): m/z=488/490/492 (M+H)⁺. LC retention time: 2.38 min.

### Example 63

### 2-Chloro-5-methoxy-N-(2-{3-[(2-hydroxy-ethyl)-isopropyl-carbamoyl]-phenylamino}-pyrimidin-5-yl)-benzamide (43)

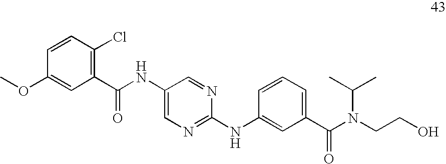

Employing a procedure similar to that used for compound 40 described in Example 56 with acid intermediate 39 described in Example 53 (30 mg, 0.16 mmol), oxalyl chloride (0.022 mL, 0.25 mmol), and amine intermediate 34 described in Example 48 (45 mg, 0.14 mmol) afforded the title compound as a pale-yellow solid (7.5 mg, 11%). MS (ES+): m/z=484/486 (M+H)⁺. LC retention time: 2.38 min.

### Example 64

### 2-Chloro-5-hydroxy-N-(2-{3-[(2-hydroxy-ethyl)-isopropyl-carbamoyl]-phenylamino}-pyrimidin-5-yl)-benzamide (XX)

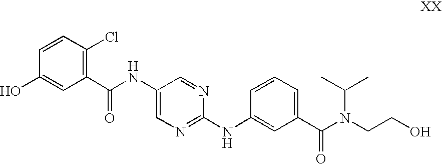

Employing a procedure similar to that described for compound XVI using compound 43 described in Example 63 (7.5 mg, 0.015 mmol) and two additions of BBr₃ (14.7 μL, 0.15 mmol and 29.4 μL, 0.31) in 0.5 mL DCM over 2 hours afforded the title compound as a pale-yellow solid (6 mg, 88%).

¹H NMR (DMSO-d₆) δ 1.11 (bs, 3H), 3.92 (bs, 2H), 6.88 (d, J=8.4 Hz, 1H), 6.90 (dd, J=8.6, 3.0 Hz, 1H), 6.96 (d, J=3.0 Hz, 1H), 7.30-7.36 (m, 2H), 7.75 (s, 1H), 7.77 (s, 1H), 8.79 (s, 2H), 9.80 (s, 1H), 10.05 (bs, 1H), 10.53 (s, 1H). MS (ES+): m/z=471/473 (M+H)⁺. LC retention time: 2.11 min

### Example 65

### 2,6-Dichloro-N-(2-{4-[(2-hydroxy-ethyl)-isopropyl-carbamoyl]-phenylamino}-pyrimidin-5-yl)-benzamide (XXI)

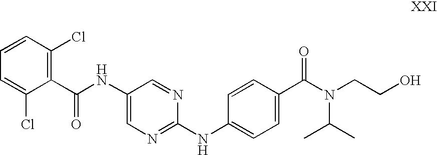

Employing a procedure similar to that described for compound XIV described in Example 54, using amine intermediate 38 described in Example 52 (57 mg, 0.18 mmol), 2,6-dichlorobenzoylchloride (0.026 mL, 0.18 mmol), and TEA (0.063 mL, 0.45 mmol) in THF (1.5 mL) afforded the title compound as an off-white solid (46 mg, 51%).

¹H NMR (DMSO-d₆) δ 1.12 (bs, 6H), 3.51 (bs, 2H), 4.73 (t, J=5.5 Hz, 1H), 7.28 (d, J=8.6 Hz, 2H), 7.53 (dd, J=9.1, 7.1 Hz, 1H), 7.61 (d, J=7.7 Hz, 2H), 7.80 (d, J=8.7 Hz, 2H), 8.79 (s, 2H), 9.93 (s, 1H), 10.91 (s, 1H). MS (ES+): m/z=488/490/492 (M+H)⁺. LC retention time: 2.34 min.

### Example 66

### 2-Chloro-5-hydroxy-N-(2-{4-[(2-hydroxy-ethyl)-isopropyl-carbamoyl]-phenylamino}-pyrimidin-5-yl)-benzamide (XXII)

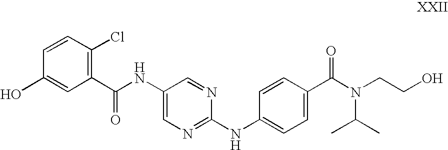

Employing a procedure similar to that used for compound 40 described in Example 56, using acid intermediate 39 described in Example 53 (68 mg, 0.36 mmol), oxalyl chloride (49.9 μL, 0.58 mmol), and amine intermediate 38 described in Example 52 (115 mg, 0.37 mmol) afforded 2-chloro-5-methoxy-N-(2-{4-[(2-hydroxy-ethyl)-isopropyl-carbamoyl]-phenylamino}-pyrimidin-5-yl)-benzamide as a pale-yellow solid. A procedure similar to that employed for compound XVI described in Example 57, using the crude solid, and BBr₃ (0.345 mL, 3.6 mmol), in DCM (20 mL) was used to achieve the title compound. The reaction was quenched with NaHCO₃ and the organic layer was separated and concentrated in vacuo. The crude mixture was purified by HPLC to afford the title compound as a pale-yellow solid (15.5 mg, 9%).

¹H NMR (DMSO-d₆) δ 1.12 (bs, 6H), 3.28-3.33 (m, 2H), 6.90 (dd, J=8.7, 3.0 Hz, 1H), 6.96 (d, J=2.9 Hz, 1H), 7.27 (d, J=8.6 Hz, 2H), 7.35 (d, J=8.8 Hz, 1H), 7.79 (d, J=8.7 Hz, 2H), 8.80 (s, 2H), 9.87 (s, 1H), 10.04 (s, 1H), 10.54 (s, 1H). MS (ES+): m/z 470/472 (M+H)⁺. LC retention time: 2.07 min.

### Example 67

### 4-Bromo-N-(2-pyrrolidin-1-yl-ethyl)-benzamide (44)

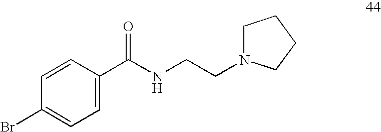

To 4-bromobenzoic acid (5 g, 24.8 mmol) in dichloromethane (125 mL) was added thionyl chloride (18.15 mL, 248.7 mmol) followed by DMF (1 mL). The reaction mixture was heated under reflux for 5 h till no gas evolution observed. The volatiles were evaporated under reduced pressure, and the residue was taken in hexane-ethyl acetate (200 mL, 3:1). The slurry was filtered through a small plug of silica gel and evaporated. The crude chloride was obtained as a yellow syrup, that eventually becomes solid (4.47 g, 82%). To the acid chloride (2.0 g, 9.11 mmol) in dichloromethane (50 mL) was added triethylamine (6.35 mL, 45.55 mmol) and pyrrolidine ethyl amine (1.15 mL, 9.11 mmol) at 0° C. and warmed to room temperature. After stirring at room temperature for 16 h, the reaction mixture was quenched with saturated aqueous sodium bicarbonate (30 mL). The organic layer was separated, and the aqueous layer was extracted again with dichloromethane (100 mL). The combined organic phase was separated, dried (MgSO₄), filtered through a silica plug, and the volatiles were removed under reduced pressure, to give a white solid (2.4 g, 89%).

### Example 68

### 4-(5-Amino-pyrimidin-2-ylamino)-N-(2-pyrrolidin-1-yl-ethyl)-benzamide (45)

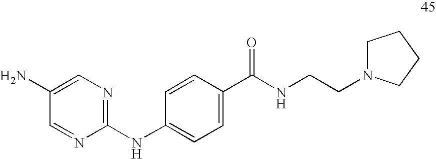

A mixture of 2-amino-5-nitropyrimidine (140 mg, 1.0 mmol), compound 44 described in Example 67 (297 mg, 1.0 mmol), Pd₂(dba)₃ (9.0 mg, 0.01 mmol), Xantphos (12 mg, 0.02 mmol) and cesium carbonate (650 mg, 2.0 mmol) were suspended in dioxane (15 mL) and heated at reflux under the argon atmosphere for 15 h. The solvent evaporated and the residue triturated with chloroform-water-brine (50 mL, 1:1:1). The chloroform layer was separated, dried, and evaporated. The residue (400 mg) was taken in methanol (50 mL) and was hydrogenated over Pd/C (10%, 120 mg) for 3 hr. The catalyst was removed by filtration, and the solvent evaporated. The residue was crystallized using chloroform-methanol mixture to give the title compound (344 mg, quant) as yellow solid.

### Example 69

### 2-Bromo-5-methoxy-N-{2-[4-(2-pyrrolidin-1-yl-ethylcarbamoyl)-phenylamino]-pyrimidin-5-yl}-benzamide (46)

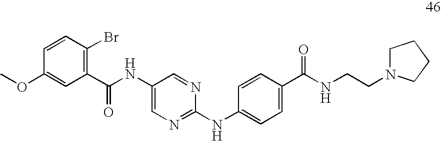

To intermediate 45 described in Example 68 (50 mg, 0.15 mmol) in THF (1 mL) was added 2-bromo-5-methoxybenzoyl chloride (45 mg, 0.18 mmol) in 1 mL THF in a single portion, and the reaction was stirred for 2 h at which point ether was added to complete precipitation of product. The reaction was filtered to afford the HCl salt of the title compound as a light-yellow solid (58 mg, 65%). MS (ES+): m/z=539/541 (M+H)⁺. LC retention time: 2.03 min.

### Example 70

### 2-Bromo-5-hydroxy-N-{2-[4-(2-pyrrolidin-1-yl-ethylcarbamoyl)-phenylamino]-pyrimidin-5-yl}-benzamide (XXIII)

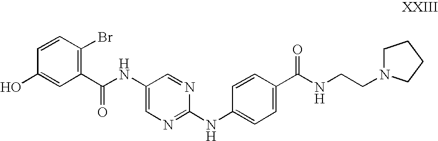

To a suspension of the HCl salt of compound 46 described in Example 69 (58 mg, 0.1 mmol) in DCM (2 mL) was added 57 μL of BBr₃ (0.6 mmol). After 20 min, the reaction was quenched with MeOH and water and concentrated in vacuo. The crude residue was purified by HPLC to afford the TFA salt of the title compound as an off-white solid (23 mg, 36%).

¹H NMR (500 MHz, DMSO-d₆) δ 1.82-1.92 (m, 2H), 1.97-2.07 (m, 2H), 3.02-3.11 (m, 2H), 3.29-3.35 (m, 2H), 3.54-3.59 (m, 2H), 3.60-3.68 (m, 2H), 6.85 (dd, J=8.7, 2.9 Hz, 1H), 6.95 (d, J=2.9, Hz, 1H), 7.49 (d, J=8.8 Hz, 1H), 7.81 (d, J=9.0 Hz, 2H), 7.86 (d, J=8.9 Hz, 2H), 8.53 (t, J=5.5 Hz 1H), 8.83 (s, 2H), 9.42 (bs, 1H), 10.02 (s, 1H), 10.09 (s, 1H), 10.57 (s, 1H). MS (ES+): m/z=525/527 (M+H)⁺. LC retention time: 1.79 min.

### Example 71

### 4-[5-(3-Hydroxymethyl-phenylamino)-pyrimidin-2-ylamino]-N-(2-pyrrolidin-1-yl-ethyl)-benzamide (XXIV)

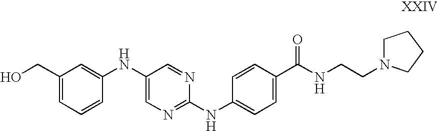

A suspension of amine intermediate 45 described in Example 68 (18.4 μL, 0.15 mmol), 3-bromobenzyl alcohol (48.5 mg, 0.15 mmol), KOtBu (40.6 mg, 0.36 mmol), Xantphos (23.4 mg, 0.04 mmol), and Pd(OAc)₂ (4.5 mg, 0.02 mmol) in dioxane (3 mL) was purged with argon for 5 min, and was heated to reflux for 2.5 hours under argon. Dioxane was removed in vacuo, and the crude mixture was purified by HPLC to afford the TFA salt of the title compound as a glassy solid (11.3 mg, 14%).

¹H NMR (DMSO-d₆) δ 1.82-1.92 (m, 2H), 1.97-2.07 (m, 2H), 3.02-3.11 (m, 2H), 3.50-3.62 (m, 4H), 3.63-3.67 (m, 2H), 4.42 (s, 2H), 6.72 (d, J=7.4 Hz, 1H), 6.79 (dd, J=8.0 Hz, J=1.9 Hz, 1H), 6.92 (s, 1H), 7.15 (t, J=7.8 Hz, 1H), 7.80 (d, J=9.0 Hz, 2H), 7.85 (d, J=9.0 Hz, 2H), 7.98 (s, 1H), 8.42 (s, 2H), 8.51 (t, J=5.7 Hz, 1H), 9.45 (bs, 1H), 9.81 (s, 1H). MS (ES+): m/z=433 (M+H)⁺. LC retention time: 1.72 min.

### Example 72

### 2-Chloro-5-hydroxy-N-{2-[4-(2-pyrrolidin-1-yl-ethylcarbamoyl)-phenylamino]-pyrimidin-5-yl}-benzamide (47)

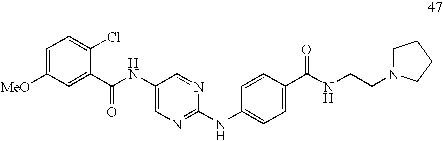

A mixture of compound 45 described in Example 68 (344 mg, 0.95 mmol), 2-chloro-5-methoxybenzoic acid (176 mg, 0.95 mmol) and DIPEA (827 μL, 4.75 mmol) were dissolved in DMF (5 mL) and treated with HATU (433 mg, 1.14 mmol) at room temperature for 16 h. The reaction mixture was extracted was triturated with ethyl acetate-water-brine (30 mL, 1:1:1). The organic layer was separated, dried (Na₂SO₄) and evaporated. The residue was purified by HPLC to give title compound as a brown solid (317 mg, 63%).

### Example 73

### 2-Chloro-5-hydroxy-N-{2-[4-(2-pyrrolidin-1-yl-ethylcarbamoyl)-phenylamino]-pyrimidin-5-yl}-benzamide (XXV)

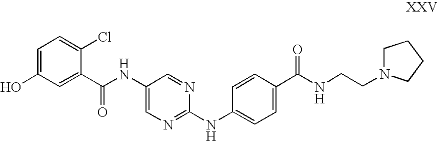

To compound 47 described in Example 72 (27 mg, 0.05 mmol) in 10 mL dichloromethane was added 1M dichloromethane solution of boron tribromide (0.5 mmol, 0.5 mL). The reaction mixture was stirred for 2 h. One more batch of boron tribromide (0.5 mL of 1M solution in dichloromethane) was added and stirring continued for another 2 h at room temperature. The reaction mixture was evaporated, the residue was dissolved in DMSO. The product was separated by prep HPLC to give the title compound as a brown syrup (10 mg, 37%).

¹H NMR (MeOH-d₄): δ 2.03-2.12 (m, 2H), 2.16-2.27 (m, 2H), 3.15-3.24 (m, 2H), 3.44 (t, J=6.0 Hz, 2H), 3.75 (t, J=5.8 Hz, 2H), 3.77-3.86 (m, 2H), 6.91 (dd, J=8.7 Hz, J=3.0 Hz, 1H), 7.00 (d, J=2.9 Hz, 1H), 7.32 (d, J=8.8 Hz, 1H), 7.85-7.92 (m, 4H), 8.81 (s, 2H). MS (ES+): m/z=481 (M+H)⁺.

### Example 74

### 2-Chloro-5-hydroxy-N-{2-[4-(2-pyrrolidin-1-yl-ethylcarbamoyl)-phenylamino]-pyrimidin-5-yl}-benzamide (XXVI)

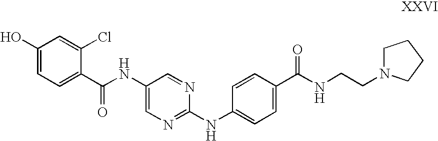

To the amine 45 described in Example 68 (35 mg, 0.1 mmol) in 2 mL of DMF, was added 2-chloro-4-hydroxybenzoic acid (19 mg, 0.1 mmol) and diisopropyl ethyl amine (41 μL, 0.3 mmol). The mixture was cooled in ice bath, and HATU (49 mg, 0.13 mmol) was added. The reaction was stirred overnight at room temperature. The crude product was separated using prep HPLC to give a cream colored solid (2.0 mg, 12%).

¹H NMR (MeOH-d₄): δ 1.27-1.41 (m, 6H), 2.06-2.09 (m, 2H), 2.29 (s, 3H), 3.18-3.28 (m, 2H), 3.65-3.73 (m, 2H), 6.83 (dd, J=8.5 Hz, J=2.3 Hz, 1H), 6.92 (d, J=2.3 Hz, 1H), 7.49 (d, J=8.6 Hz, 1H), 7.82-7.9 (m, 4H), 8.80 (s, 2H). MS (ES+): m/z=481 (M+H)⁺.

### Example 75

### 3-Hydroxy-2-methyl-N-{2-[4-(2-pyrrolidin-1-yl-ethylcarbamoyl)-phenylamino]-pyrimidin-5-yl}-benzamide (XXVII)

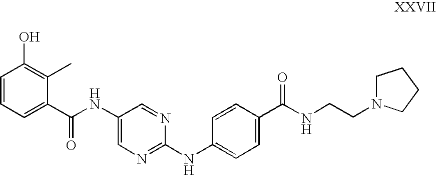

To the amine 45 described in Example 68 (50 mg, 0.15 mmol) in 10 mL THF was added 3-acetoxy-2-methylbenzoyl chloride (21 mg, 0.12 mmol) and diisopropyl ethyl amine (78 μL, 0.45 mmol). The reaction mixture was stirred at room temperature for 2 h followed by 6 h. reflux. The solvent evaporated and the residue was dissolved in 5 mol of anhydrous methanol. The methanol solution was treated with 1 mL of 25% methanolic solution of sodium methoxide for 15 minutes. The solvent evaporated and the residue was dissolved in 2 mL of DMSO, and separated using prep HPLC to give a cream colored solid (29 mg, 34%).

¹H NMR (MeOH-d₄): δ 2.02-2.12 (m, 2H), 2.16-2.27 (m, 2H), 2.29 (s, 314), 3.14-3.23 (m, 214), 3.44 (t, J=6.0 Hz, 214), 3.75 (t, J=5.6 Hz, 214), 3.77-3.86 (m, 214), 6.91 (dd, J=8.0 Hz, J=0.8 Hz, 1H), 6.98 (dd, J=7.7 Hz, J=0.9 Hz, 1H), 7.13 (t, J=7.7 Hz, 1H), 8.82 (s, 2H). MS (ES+): m/z=461 (M+H)⁺.

### Example 76

### 2,6-Dichloro-N-{2-[4-(2-pyrrolidin-1-yl-ethylcarbamoyl)-phenylamino]-pyrimidin-5-yl}-benzamide (XXVIII)

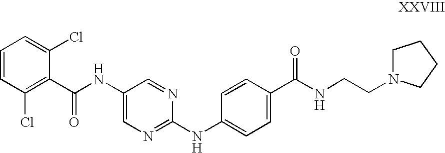

To the amine 45 described in Example 68 (32 mg, 0.1 mmol) in 10 mL THF was added 2,6-dichlorobenzoyl chloride (16 μL, 0.11 mmol) and diisopropyl ethyl amine (52 μL, 0.3 mmol). The reaction mixture was refluxed overnight. The solvent evaporated and the residue was dissolved in DMSO, and separated using prep HPLC to give a green/yellow solid (7 mg, 12%).

¹H NMR (MeOH-d₄): δ 2.02-2.12 (m, 2H), 2.16-2.27 (m, 2H), 3.14-3.23 (m, 2H), 3.44 (t, J=5.9 Hz, 2H), 3.75 (t, J=5.7 Hz, 2H), 3.77-3.86 (m, 2H), 7.47 (dd, J=7.7 Hz, J=6.8 Hz, 114), 7.85-7.92 (m 4H), 8.81 (s, 2H). MS (ES+): m/z=499 (M+H)⁺.

### Example 77

### 2,6-Dimethyl-N-{2-[4-(2-pyrrolidin-1-yl-ethylcarbamoyl)-phenylamino]-pyrimidin-5-yl}-benzamide (XXIX)

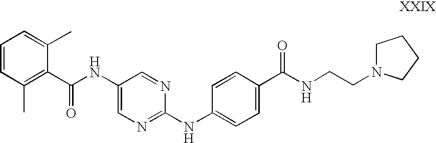

To the amine 45 described in Example 68 (35 mg, 0.1 mmol) in 10 mL THF was added 2,6-dimethylbenzoyl chloride (21 mg, 0.12 mmol) and diisopropyl ethyl amine (52 μL, 0.3 mmol). The reaction mixture was refluxed overnight. The solvent evaporated and the residue was dissolved in DMSO, and separated using prep HPLC to give a brown syrup (6 mg, 12%).

¹H NMR (MeOH-d₄): δ 2.01-2.12 (m, 2H), 2.15-2.25 (m, 2H), 2.39 (s, 6H), 3.14-3.22 (m, 2H), 3.44 (t, J=5.8 Hz, 2H), 3.75 (t, J=5.8 Hz, 2H), 3.76-3.85 (m, 2H), 7.13 (d, J=7.7 Hz, 2H), 7.25 (t, J=7.6 Hz, 1H), 7.87 (dd, J=8.1 Hz, J=6.6 Hz, 4H), 8.83 (s, 2H). MS (ES+): m/z=460 (M+H)⁺.

### Example 78

### 2-[4-(2-Pyrrolidin-1-yl-ethoxy)-phenylamino]-4-trifluoromethyl-pyrimidine-5-carboxylic acid ethyl ester (48)

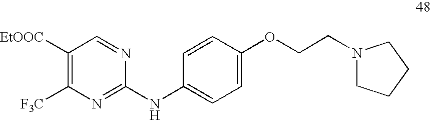

A mixture of 2-amino-4-trifluoromethyl-pyrimidine-5-carboxylic acid ethyl ester (280 mg, 1.2 mmol), 1-[2-(4-bromophenoxy)ethyl]pyrrolidine (640 mg, 2.4 mmol), cesium carbonate (1.16 g, 3.6 mmol), and Xantphos (140 mg, 0.24 mmol), Pd₂(dba)₃ (110 mg, 0.12 mmol) in 20 mL of anhydrous dioxane was degassed with argon for 5 minutes and was refluxed overnight under argon. After cooling down, the solvent was removed under reduced pressure. The crude product was purified silica gel column (3.5×16 cm) chromatography using 20% CH₃OH in CHCl₃ as an eluent to give a pale yellow solid (300 mg, 59%).

### Example 79

### 2-[4-(2-Pyrrolidin-1-yl-ethoxy)-phenylamino]-4-trifluoromethyl-pyrimidine-5-carbonyl chloride (49)

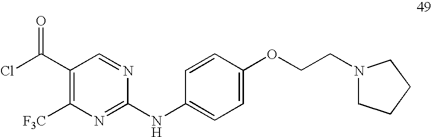

A solution of compound 48 described in Example 78(250 mg, 0.59 mmol) and KOH (330 mg, 5.9 mmol) in EtOH (20 mL) was refluxed for 5 h. TLC showed no starting material. The solvent was removed under reduced pressure. The crude material was dissolved in water (5 ml) and acidified with aqueous HBr to pH 2 to get yellow precipitate. The solid was collected by filtration, washed by water and dried in vacuo to give a pale yellow solid (180 mg, 77%). The crude carboxylic acid (80 mg, 0.20 mmol) was dissolved in 2.0 M thionyl chloride in dichloromethane (20 mL, 40 mmol). The reaction mixture was refluxed under argon for 4 h. The volatiles were removed under vacuum and the crude product was dried at high vacuum overnight.

### Example 80

### 2-[4-(2-Pyrrolidin-1-yl-ethoxy)-phenylamino]-4-trifluoromethyl-pyrimidine-5-carboxylic acid (2-chloro-5-hydroxy-phenyl)-amide (XXX)

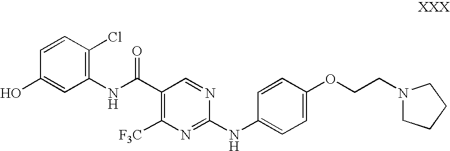

The crude acid chloride 49 described in Example 79 was dissolved in anhydrous toluene (10 mL) and was treated with 3-amino-4-chlorophenol (140 mg, 1 mmol) under reflux for 2 h under argon. The crude product was purified by silica gel column chromatography using 20% CH₃OH in CHCl₃ as an eluent. The similar fractions were combined and the solvent was removed under reduced pressure to give the title compound as a pale yellow solid (80 mg, 64%).

¹H NMR (DMSO-d₆): δ 1.69 (m, 4H), 2.54 (m, 4H), 2.80 (m, 2H), 4.05 (t, J=5.9 Hz, 2H), 6.66 (dd, J=8.7 Hz, J=2.8 Hz, 1H), 6.94 (d, J=9.0 Hz, 2H), 7.29 (d, J=8.8 Hz, 2H), 7.62 (d, J=9 Hz, 2H), 8.83 (s, 1H), 9.82 (s, 1H), 10.09 (s, 1H), 10.32 (s, 1H). MS (ES+): m/z=522 (M+H)⁺.

### Example 81

### 1-Bromo-4-(3-bromo-propane-1-sulfonyl)-benzene (50)

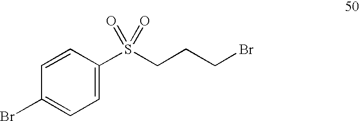

To a solution of 4-bromothiophenol (4.0 g, 21.2 mmol) in methanol (50 mL) was added NaOMe (2.28 g, 42 mmol). The mixture was stirred at room temperature until clear. The clear solution was added dropwise to 22 mL of 1,3-dibromopropane (42.5 g, 210 mmol) at room temperature. The reaction mixture was stirred at room temperature for 16 h and diluted with 20 mL of dichloromethane (CH₂Cl₂) and 50 mL of water. The combined organic phase were dried (MgSO₄) and the volatiles were removed under reduced pressure. To the crude product in 150 mL CH₂Cl₂ was added 3-chloroperoxybenzoic acid (4.9 g, 20 mmol) at 0° C. After stirring at the same temperature for 1 h, another batch of mCPBA (4.9 g, 20 mmol) was added. The stirring was continued for 30 min at 0° C. before the mixture was allowed to warm to room temperature. It was diluted with CH₂Cl₂ (40 mL) and washed twice with saturated aqueous NaHCO₃ solution. The organic phase was dried (MgSO₄) and the product was purified by silica gel column chromatography to give the title compound as a colorless solid (5.35 g, 76%). R_{f}=0.50 (EtOAc/hexanes=1/1).

### Example 82

### 1-[3-(4-Bromo-benzenesulfonyl)-propyl]-pyrrolidine (51)

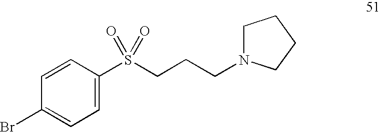

To intermediate 50 described in Example 81 (1.0 g, 3.27 mmol) in 20 mL anhydrous 1.4-dioxane were added Cs₂CO₃ (2.13 g, 6.54 mmol) and pyrrolidine (540 μL, 6.54 mmol). The reaction mixture was stirred for 16 h at room temperature. The reaction mixture was diluted with EtOAc (100 mL) and washed with saturated sodium bicarbonate solution The combined organic phase were dried (Na₂SO₄), and the solvent was evaporated. The product was dried in vacuo to give a brown oil 1 (994 mg, 91%), which was used without further purification.

¹H NMR (DMSO-d₆): δ 1.62 (m, 4H), 1.65-1.70 (m, 2H), 2.32 (m, 2H), 2.39 (t, J=7.0 Hz, 2H), 3.34 (m, 2H), 7.83 (d, J=9.0 Hz, 2H), 7.88 (d, J=9.0 Hz, 1H). MS (ES+): m/z=333 (M+H)⁺.

### Example 83

### N-[4-(3-Pyrrolidin-1-yl-propane-1-sulfonyl)-phenyl]-pyrimidine-2,5-diamine (52)

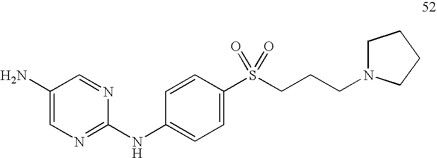

To a solution of 2-amino-5-nitropyrimidine (350 mg, 2.5 mmol) in 20 mL anhydrous 1,4-dioxane were added intermediate 51 described in Example 82 (1.25 g, 3.76 mmol) in 5 mL anhydrous 1,4-dioxane, Xantphos, (289 mg, 0.5 mmol), Pd₂(dba)₃ (229 mg, 0.25 mmol) and Cs₂CO₃ (1.63 g, 5 mmol). The reaction mixture was stirred at 100° C. for 5 h under argon. The reaction mixture was diluted with methanol and CH₂Cl₂ (5 mL each) then filtered. The filtrate was washed with brine. The organic phase was dried (Na₂SO₄) and the solvent was removed. The residue was dissolved in methanol and ethyl acetate (2 mL each) and diluted with 20 mL hexane. The precipitated yellow-brown solid, was isolated by filtration and dried in vacuo (800 mg). The crude product was hydrogenated in 20 mL methanol using Pd/C (10%, 500 mg) for 2 h. The palladium catalyst was removed by filtration, and the solvent was evaporated. The residue was dried in vacuo to yield the title compound (550 mg, 73%), which was used without further purification.

¹H NMR (DMSO-d₆): δ 1.60-1.68 (m, 6H), 2.32 (m, 4H), 2.39 (m, 2H), 3.19 (m, 2H), 5.02 (bs, 2H), 7.68 (d, J=9.0 Hz, 2H), 7.87 (d, J=9.0 Hz, 1H), 8.02 (s, 2H), 9.68 (s, 1H). MS (ES+): m/z=362 (M+H)⁺.

### Example 84

### 3-Cyano-N-{2-[4-(3-pyrrolidin-1-yl-propane-1-sulfonyl)-phenylamino]-pyrimidin-5-yl}-benzamide(XXXI)

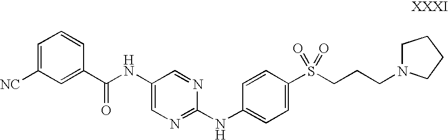

To a solution of intermediate 52 described in Example 83 (60 mg, 0.166 mmol) and 3-cyanobenzoic acid (49 mg, 0.332 mmol) in 15 mL acetonitrile was added ethylene carbodiimide (EDC) (64 mg, 0.332 mmol). The reaction mixture was stirred at room temperature for 16 h, and the solvent was removed. The residue was dissolved in 20 mL CH₂Cl₂ and washed with aqueous saturated NaHCO₃ solution (20 mL). The aqueous layer was extracted with CH₂Cl₂ (50 mL). The combined organic phase were dried (Na₂SO₄) and the solvent was removed. The crude product was purified by reverse phase preparative HPLC to give the title compound as a white solid (60 mg, 60%).

¹H NMR (DMSO-d₆): δ 1.83 (m, 2H), 1.92 (m, 2H), 1.99 (m, 2H), 2.97 (m, 2H), 3.21 (m, 2H), 3.34 (t, J=7.6 Hz, 2H), 3.53 (m, 2H), 7.80 (dd, J=8.0 Hz, J=7.8 Hz, 1H), 7.81 (d, J=8.9 Hz, 2H), 8.04 (d, J=8.9 Hz, 2H), 8.11 (ddd, J=7.8 Hz, J=1.5 Hz, J=1.5 Hz, 1H), 8.28 (ddd, J=8.0 Hz, J=1.5 Hz, J=1.5 Hz, 1H), 8.42 (dd, J=1.5 Hz, J=1.5 Hz, 1H), 8.92 (s, 2H), 10.33 (s, 1H), 10.65 (s, 1H). MS (ES+): m/z=491 (M+H)⁺.

### Example 85

### 2-Chloro-5-methoxy-N-{2-[4-(3-pyrrolidin-1-yl-propane-1-sulfonyl)-phenylamino]-pyrimidin-5-yl}-benzamide (53)

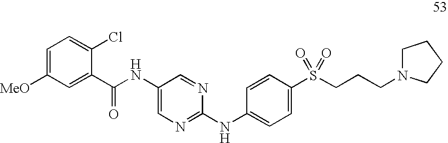

To a solution of intermediate 52 described in Example 83 (108 mg, 0.3 mmol) and 2-chloro-5-methoxybenzoic acid (112 mg, 0.6 mmol) in 20 mL acetonitrile was added EDC (115 mg, 0.6 mmol). The reaction mixture was stirred at room temperature for 16 h and the solvent was removed. The residue was dissolved in 20 mL CH₂Cl₂ and washed with aqueous saturated NaHCO₃ solution (20 mL). The aqueous layer was extracted with CH₂Cl₂ (20 mL). The combined organic phase was dried (Na₂SO₄) and the solvent was evaporated. The crude product was purified by reverse phase preparative HPLC to give the title compound as an off-white solid (50 mg, 26%).

¹H NMR (DMSO-d₆): δ 1.82 (m, 2H), 1.92 (m, 2H), 1.99 (m, 2H), 2.97 (m, 2H), 3.21 (m, 2H), 3.34 (t, J=7.6 Hz, 2H), 3.53 (m, 2H), 3.82 (s, 3H), 7.11 (dd, J=8.9 Hz, J=3.0 Hz, 1H), 7.21 (d, J=3.0 Hz, 1H), 7.49 (d, J=8.9 Hz, 1H), 7.80 (d, J=9.0 Hz, 2H), 8.03 (d, J=9.0 Hz, 2H), 8.89 (s, 2H), 10.33 (s, 1H), 10.69 (s, 1H). MS (ES+): m/z=531 (M+H)⁺.

### Example 86

### 2-Chloro-5-hydroxy-N-{2-[4-(3-pyrrolidin-1-yl-propane-1-sulfonyl)-phenylamino]-pyrimidin-5-yl}-benzamide (XXXII)

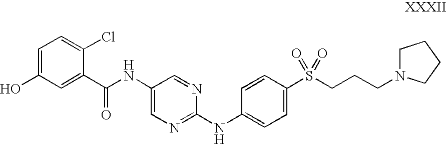

To a solution of compound 53 described in Example 85 (42 mg, 0.065 mmol) in 3 mL anhydrous CH₂Cl₂ was added BBr₃ (31 μl, 0.33 mmol). The mixture was stirred at room temperature for 1 h and was poured into saturated aqueous sodium thiosulfate. The product was extracted with 20 mL CH₂Cl₂/methanol (90:10). The combined organic phases was dried (Na₂SO₄), and the solvent was evaporated. The crude product was purified by reverse phase preparative HPLC to give the title compound as a white solid (29 mg, 71%).

¹H NMR (DMSO-d₆): δ 1.82 (m, 2H), 1.92 (m, 2H), 1.99 (m, 2H), 2.97 (m, 2H), 3.21 (m, 2H), 3.34 (t, J=7.7 Hz, 2H), 3.53 (m, 2H), 6.92 (dd, J=8.7 Hz, J=2.9 Hz, 1H), 6.96 (d, J=2.9 Hz, 1H), 7.36 (d, J=8.7 Hz, 1H), 7.80 (d, J=9.0 Hz, 2H), 8.03 (d, J=9.0 Hz, 2H), 8.88 (s, 2H), 10.09 (s, OH), 10.32 (s, 1H), 10.65 (s, 1H). MS (ES+): m/z=516 (M+H)⁺.

### Example 87

### 3-Hydroxy-2-methyl-N-{2-[4-(3-pyrrolidin-1-yl-propane-1-sulfonyl)-phenylamino]-pyrimidin-5-yl}-benzamide (XXXIII)

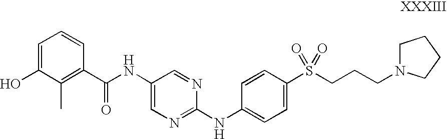

To a solution of intermediate 52 described in Example 83 (47 mg, 0.13 mmol) in 5 mL anhydrous THF was added a solution of 3-acetoxy-2-methylbenzoyl chloride (33.2 mg, 0.156 mmol) in 5 mL anhydrous THF. The mixture was stirred at room temperature for 40 h and the solvent was removed. The residue in 5 mL methanol was treated with 25% w/w NaOMe in methanol (250 mg, 1.16 mmol) for 2 h. The reaction mixture was quenched with brine (10 mL) and the crude product was extracted with CH₂Cl₂ (50 mL). The combined organic phase was dried (MgSO₄) and the solvent was removed. The crude product was purified by reverse phase preparative HPLC to give the title compound as an off-white solid (20 mg, 25%).

¹H NMR (DMSO-d₆): δ 1.82 (m, 2H), 1.92 (m, 2H), 1.99 (m, 2H), 2.97 (m, 2H), 3.21 (m, 2H), 3.34 (t, J=7.6 Hz, 2H), 3.53 (m, 2H), 6.92-6.95 (m, 2H), 7.12 (t, J=7.8 Hz, 1H), 7.80 (d, J=9.0 Hz, 2H), 8.03 (d, J=9.0 Hz, 2H), 8.90 (s, 2H), 9.66 (s, OH), 10.29 (s, 1H), 10.42 (s, 1H). MS (ES+): m/z=496 (M+H)⁺.

### Example 88

### 2,6-Dichloro-N-{2-[4-(3-pyrrolidin-1-yl-pronane-1-sulfonyl)-phenylamino]-pyrimidin-5-yl}-benzamide (XXXIV)

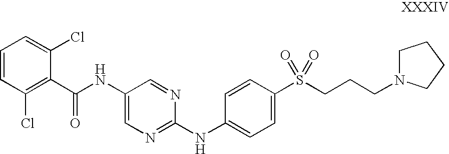

A solution of 2,6-dichlorobenzoylchloride (46 mg, 0.22 mmol) in 2 mL anhydrous THF was added dropwise to a solution of intermediate 52 described in Example 83 (65 mg, 0.18 mmol) in 3 mL anhydrous THF. The mixture was stirred at room temperature for 40 h. The solvent was removed and the crude product was purified by reverse phase preparative HPLC to give the title compound as an off-white solid (14 mg, 12%).

¹H NMR (DMSO-d₆): δ 1.82 (m, 2H), 1.92 (m, 2H), 1.99 (m, 2H), 2.97 (m, 2H), 3.21 (m, 2H), 3.34 (t, J=7.7 Hz, 2H), 3.53 (m, 2H), 7.54 (dd, J=8.7 Hz, J=7.2 Hz, 1H), 7.62 (d, J=8.7 Hz, 2H), 7.80 (d, J=8.9 Hz, 2H), 8.03 (d, J=8.9 Hz, 2H), 8.87 (s, 2H), 10.38 (s, 1H), 11.02 (s, 1H). MS (ES+): m/z=534 (M+H)⁺.

### Example 89

### 2-Chloro-4-hydroxy-N-{2-[4-(3-pyrrolidin-1-yl-propane-1-sulfonyl)-phenylamino]-pyrimidin-5-yl}-benzamide (XXXV)

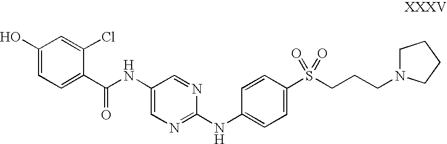

To a solution of 4-benzyloxy-2-chloro-benzoic acid (58 mg, 0.22 mmol) in 2 mL anhydrous CH₂Cl₂ were added CDMT (44 mg, 0.25 mmol) and NMM (66 μL, 0.6 mmol). After stirring for 1 h at room temperature, intermediate 52 described in Example 83 (72 mg, 0.2 mmol) was added and stirring was continued for 16 h. The solvent was removed and the crude product was purified by reverse phase preparative HPLC. The purified precursor was dissolved in 1 mL anhydrous CH₂Cl₂ and was treated with BBr₃ (15.1 μL, 0.16 mmol) for 1 h at 0° C. The reaction mixture was diluted with 10 mL CH₂Cl₂ and washed twice with saturated aqueous sodium thiosulfate solution (10 mL). The organic phase was dried (Na₂SO₄), and the solvent was evaporated. The crude product was purified by reverse phase preparative HPLC to afford the title compound as a white solid (5 mg, 4%).

¹H NMR (MeOH-d₄): δ 2.02 (m, 2H), 2.11-2.18 (m, 4H), 3.09 (m, 2H), 3.27-3.37 (m, 4H), 3.66 (m, 2H), 6.84 (dd, J=8.5 Hz, J=2.2 Hz, 1H), 6.93 (d, J=2.2 Hz, 1H), 7.49 (d, J=8.5 Hz, 1H), 7.86 (d, J=9.0 Hz, 2H), 8.05 (d, J=9.0 Hz, 2H), 8.85 (s, 2H). MS (ES+): m/z=516 (M+H)⁺.

### Example 90

### 3-Hydroxy-N-{2-[4-(3-pyrrolidin-1-yl-propane-1-sulfonyl)-phenylamino]-pyrimidin-5-yl}-benzamide (XXXVI)

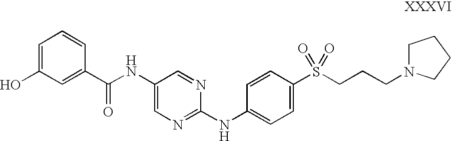

To a solution of 3-hydroxybenzoic acid (28 mg, 0.2 mmol) in 2 mL anhydrous CH₂Cl₂ were added CDMT (39 mg, 0.22 mmol) and N-methylmorpholine (44 μL, 0.4 mmol). After stirring for 1 h at room temperature, intermediate 52 described in Example 83 (65 mg, 0.18 mmol) was added in CH₂Cl₂ and DMF (1 mL each) and stirring was continued for 16 h. The solvent was removed and the crude product was purified by preparative TLC using CHCl₃/MeOH/NH₄OH (90:10:1) as the mobile phase to give the title compound as an off-white solid (8 mg, 9%).

¹H NMR (MeOH-d₄): δ 1.81 (m, 4H), 1.90 (m, 2H), 2.54 (m, 4H), 2.58 (t, J=7.5 Hz, 2H), 3.26 (m, 2H), 7.02 (d, J=8.0 Hz, 1H), 7.35 (dd, J=8.0 Hz, J=8 Hz, 1H), 7.38 (bs, 1H), 7.42 (d, J=8.0 Hz, 1H), 7.83 (d, J=9.0 Hz, 2H), 8.04 (d, J=9 Hz, 2H), 8.86 (s, 2H). MS (ES+): m/z=482 (M+H)⁺.

### Example 91

### 2,5-Dichloro-N-{2-[4-(3-pyrrolidin-1-yl-propane-1-sulfonyl)-phenylamino]-pyrimidin-5-yl}-benzamide (XXXVII)

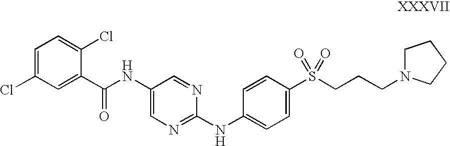

A solution of 2,5-dichlorobenzoylchloride (48 mg, 0.23 mmol) in 2 mL anhydrous THF was added dropwise to a solution of intermediate 52 described in Example 83 (65 mg, 0.18 mmol) in 3 mL anhydrous THF. The mixture was stirred at room temperature for 5 h under argon. The solvent was removed and the crude product was purified by reverse phase preparative HPLC to give the title compound as an off-white solid (12 mg, 11%).

¹H NMR (MeOH-d₄): δ 2.02 (m, 2H), 2.12-2.18 (m, 4H), 3.08 (m, 2H), 3.31-3.37 (m, 4H), 3.66 (m, 2H), 7.52-7.56 (m, 2H), 7.68 (bs, 1H), 7.86 (d, J=9.0 Hz, 2H), 8.05 (d, J=9.0 Hz, 2H), 8.86 (s, 2H). MS (ES+): m/z=534 (M+H)⁺.

### Example 92

### N²-(4-(2-(Pyrrolidin-1-yl)ethoxy)phenyl)pyrimidine-2,5-diamine (54)

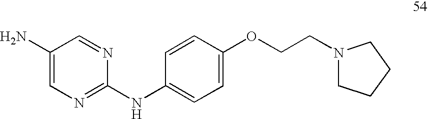

To a solution of the 2-amino-5-nitropyrimidine (0.54 g, 4 mmol) in anhydrous 1,4-dioxane (20 mL) was added 1-[2-(4-bromophenoxy)ethyl]pyrrolidine (1.62 g, 6 mmol), Cs₂CO₃ (5.2 g, 16 mmol), Pd₂(dba)₃ (0.36 g, 0.4 mmol), and Xantphos (0.7 g, 1.2 mmol). The suspension was heated under reflux for 2 h under argon. The solid was filtered off and washed with EtOAc. The filtrate was washed with brine (1×100 mL) and the aqueous was extracted with EtOAc (3×50 mL). Combined organic solution was dried (Na₂SO₄) and concentrated until 10 mL remain solution before adding hexane (100 mL). The mixture was sonicated for 2 min. The solid was collected by filtration and washed with hexane. The crude material was further purified by flash column (CH₂Cl₂:MeOH:NH₃.H₂O=100:10:1). The obtained yellow solid was dissolved in MeOH (200 mL) and bubbled with Ar for 2 min. before adding 10% Pd—C. The mixture was hydrogenated for 1 h at room temperature. The catalyst was filtered off and washed with MeOH. The filtrate was concentrated in vacuo. The desired product was obtained as a yellow solid (0.48 g, 40%).

### Example 93

### N-(2-(4-(2-(Pyrrolidin-1-yl)ethoxy)phenylamino)pyrimidin-5-yl)-2-chloro-5-hydroxybenzamide (XXXVIII)

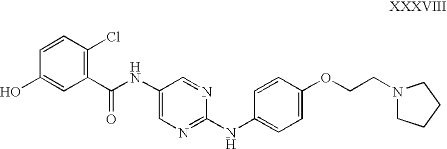

To a solution of 2-chloro-5-methoxybenzoic acid (97 mg, 0.52 mmol) in anhydrous CH₂Cl₂ (10 mL) was added 2-chloro-4,6-diimethyoxy-1,3,5-triazine (CDMT, 92 mg, 0.52 mmol), and 4-methylmopholine (NMM, 0.2 mL, 1.73 mmol). The mixture was stirred for 0.5 h at room temperature followed by adding compound 54 described in Example 92 (130 mg, 0.43 mmol). The mixture was stirred for another 2 h at room temperature. The saturated NaHCO₃ (20 mL) was added and the mixture was stirred for 5 min. The organic layer was separated and aqueous was extracted with CH₂Cl₂ (3×10 mL). The combined organic solution was dried (Na₂SO₄). The solvent was removed in vacuo and the residue was dissolved in anhydrous CH₂Cl₂ (10 mL) and 1.0 M BBr₃ in CH₂Cl₂ (3.5 mL, 3.5 mmol) was added. The reaction mixture was stirred for 4 h at room temperature. The saturated NaHCO₃ (20 ml) was added and sonicated. The product was precipitated and collected by filtration, washed with H₂O and CH₂Cl₂, to yield the final product (95 mg, 45%) as a yellow solid.

¹H NMR (DMSO-d₆): δ 1.88-191 (m, 2H), 1.99-2.01 (m, 2H), 3.08-3.11 (m, 2H), 3.54-3.58 (m, 4H), 4.32 (t, J=4.8 Hz, 2H), 6.92 (dd, J=8.7 Hz, J=2.8 Hz, 1H), 6.93 (d, J=9.0 Hz, 2H), 6.98 (d, J=2.9 Hz, 1H), 7.32 (d, J=8.7 Hz, 1H), 7.65 (d, J=9.0 Hz, 2H), 8.73 (s, 2H), 9.52 (s, 1H), 10.49 (s, 1H), 11.16 (s, 1H). MS (ES+): m/z=454 (M+H)⁺.

### Example 94

### N-(2-(4-(2-(Pyrrolidin-1-yl)ethoxy)phenylamino)pyrimidin-5-yl)-2,6-dimethyl-benzamide (XXXIX)

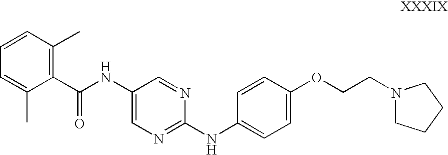

To a solution of compound 54 described in Example 92 (109 mg, 0.36 mmol) in anhydrous PhMe (6 mL) was added 2,6-dimethylbenzoyl chloride (74 mg, 0.44 mmol). The mixture was heated under reflux for 18 h. The saturated NaHCO₃ (30 mL) and CH₂Cl₂ (30 ml) were added. Organic phase was separated and aqueous was extracted with CH₂Cl₂ (3×10 mL). The combined organic solution was dried (Na₂SO₄). The product was purified by preparative HPLC; fractions containing the products were combined. EtOAc (20 ml) and saturated NaHCO₃ (20 mL) were added and organic phase was separated. The aqueous phase was extracted with EtOAc (2×10 mL). Combined organic solution was dried (Na₂SO₄) to yield the final product (33 mg, 16%) as a yellow solid.

¹H NMR (DMSO-d₆): δ 1.88-191 (m, 2H), 1.98-2.03 (m, 2H), 2.29 (s, 6H), 3.08-3.12 (m, 2H), 3.54-3.59 (m, 4H), 4.30 (t, J=4.9 Hz, 2H), 6.96 (d, J=7.0 Hz, 2H), 7.12 (d, J=7.7 Hz, 2H), 7.24 (t, J=7.6, 1H), 7.66 (d, J=7.0 Hz, 2H), 8.74 (s, 2H), 9.52 (s, 1H), 10.40 (s, 1H), 10.57 (br, 1H). MS (ES+): m/z=433 (M+H)⁺.

### Example 95

### N-(2-(4-(2-(Pyrrolidin-1-yl)ethoxy)phenylamino)pyrimidin-5-yl)-2-chloro-6-methylbenzenesulfonyl amide (XL)

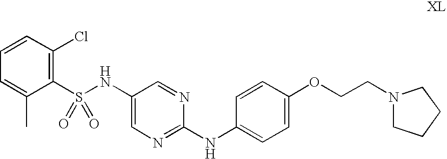

The title product was prepared by an method analogous to that described for compound XXXIX described in Example 94, except 2-chloro-6-methylbenzenesulfonyl chloride (81.2 mg. 0.36 mmol) and compound 54 described in Example 92 (90 mg, 0.3 mmol) were used to yield the HCl salt of the final product (25 mg, 13%) as a yellow solid.

¹H NMR (DMSO-d₆): 61.87-190 (m, 2H), 1.98-2.02 (m, 2H), 2.49 (s, 3H), 3.05-3.10 (m, 2H), 3.51-3.56 (m, 4H), 4.29 (t, J=4.9 Hz, 2H), 6.92 (d, J=9.2 Hz, 2H), 7.34 (d, J=7.2 Hz, 2H), 7.47 (t, J=7.6 Hz, 1H), 7.51 (d, J=7.2, 1H), 7.56 (d, J=9.2, 2H), 8.07 (s, 2H), 9.58 (s, 1H), 10.15 (s, 1H), 10.88 (br, 1H). MS (ES+): m/z=490 (M+H)⁺.

### Example 96

### 3-((2-(4-(2-(Pyrrolidin-1-yl)ethoxy)phenylamino)pyrimidin-5-ylamino)methyl)-4-chlorophenol (XLI)

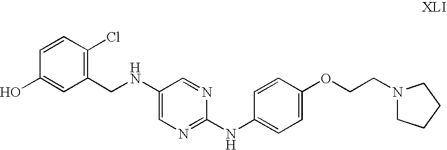

To a solution of compound 54 described in Example 92 (46 mg, 0.15 mmol) in anhydrous 1,4-dioxane (10 mL) was added Cs₂CO₃ (100 mg, 0.31 mmol), 2-chloro-5-methoxybenzyl bromide (37 mg, 0.15 mmol). The mixture was heated at 60° C. for 18 h. The solid was filtered off. The product was purified by preparative HPLC; fractions containing the products were combined. EtOAc (20 ml) and saturated NaHCO₃ (20 mL) were added and organic phase was separated. The aqueous was extracted with EtOAc (2×10 mL). Combined organic solution was dried (Na₂SO₄). The combined organic solution was dried (Na₂SO₄). The solvent was removed in vacuo and the residue was dissolved in anhydrous CH₂Cl₂ (10 mL) and 1.0 M BBr₃ in CH₂Cl₂ (3.5 mL, 3.5 mmol) was added. The reaction mixture was stirred for 4 h at room temperature. The saturated NaHCO₃ (20 ml) was added and sonicated. The organic layer was separated and aqueous was extracted with CH₂Cl₂ (3×10 mL). Combined organic solution was dried (Na₂SO₄) to yield the final product (16 mg, 21%) as a yellow solid.

¹H NMR (DMSO-d₆): δ 1.87-190 (m, 2H), 1.98-2.03 (m, 2H), 3.08-3.12 (m, 2H), 3.54-3.59 (m, 4H), 4.26 (t, J=4.8 Hz, 2H), 6.68 (dd, J=8.7 Hz, J=2.9 Hz, 1H), 6.85 (d, J=2.9 Hz, 1H), 6.89 (d, J=9.0 Hz, 2H), 7.22 (d, J=8.6 Hz, 1H), 7.58 (d, J=9.0 Hz, 2H), 7.90 (s, 2H), 8.97 (s, 1H), 9.20 (s, 1H). MS (ES+): m/z=440 (M+H)⁺.

### Example 97

### 1-[2-(3-Bromo-phenoxy)-ethyl]-pyrrolidine (55)

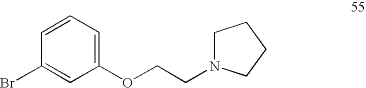

3-Bromophenol (5.34 g, 30.9 mmol) and 1-(2-chloro-ethyl)-pyrrolidine hydrochloride (5.24 g, 30.9 mmol) were combined and diluted with DMF (100 mL). Potassium carbonate (34 g, 247 mmol) was then added and the resulting mixture was allowed to stir at ambient temperature for 72 h. Reaction was then poured onto water and extracted with ethyl acetate. Organic phase was washed with brine, dried over sodium sulfate, filtered and evaporated to colorless oil. Crude product was then chromatographed to remove any unreacted bromide. Pure fractions were combined and evaporated to clear yellowish oil (3.6 g, 43%).

### Example 98

### N²-(3-(2-(Pyrrolidin-1-yl)ethoxy)phenyl)pyrimidine-2,5-diamine (56)

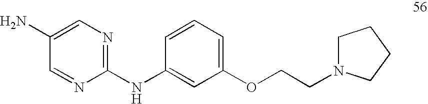

To a solution of the 2-amino-5-nitropyrimidine (200 mg, 1.4 mmol) in anhydrous 1,4-dioxane (20 mL) was added compound 55 described in Example 97 (380 mg, 1.4 mmol), Cs₂CO₃ (1.82 g, 5.6 mmol), Pd₂(dba)₃ (128 mg, 0.14 mmol), and Xantphos (243 mg, 0.42 mmol). The suspension was heated under reflux for 2 h under Ar. The solid was filtered off and washed with EtOAc. The filtrate was washed with brine (1×100 mL) and the aqueous was extracted with EtOAc (3×50 mL). Combined organic solution was dried (Na₂SO₄) and concentrated until 10 ml remain solution before adding hexane (100 mL). The mixture was sonicated for 2 min. The solid was collected by filtration and washed with hexane. The crude material was further purified by flash column (SiO₂/CH₂Cl₂, then CH₂Cl₂:MEOH:NH₃.H₂O=100:10:1). The obtained yellow solid was dissolved in MeOH (200 mL) and bubbled with Ar for 2 min before adding 10% Pd—C. The mixture was hydrogenated for 1 h at room temperature. The catalyst was filtered off and washed with MeOH. The filtrate was concentrated in vacuo. The desired product was obtained as a yellow solid (350 mg, 83%).

### Example 99

### N-(2-(3-(2-(Pyrrolidin-1-yl)ethoxy)phenylamino)pyrimidin-5-yl)-2-chloro-5-hydroxybenzamide (XLII)

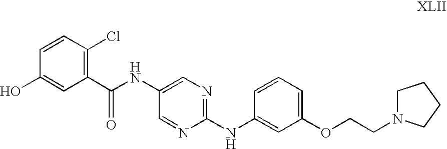

The title compound was prepared by a method analogous to that of compound XXXIX described in Example 94, except using compound 56 described in Example 98 (174 mg, 0.58 mmol) to give the title compound (80 mg, 68%) as a yellow solid. ¹H NMR (DMSO-d₆): δ 1.88-191 (m, 2H), 1.99-2.04 (m, 2H), 3.08-3.14 (m, 2H), 3.57-3.60 (m, 4H), 4.32 (t, J=4.8 Hz, 2H), 6.59 (dd, J=8.5 Hz, J=2.4 Hz, 1H), 6.92 (dd, J=8.9 Hz, J=2.9 1H), 6.97 (d, J=2.9 Hz, 1H), 7.21 (t, J=8.3 Hz, 1H), 7.31 (dd, J=8.4 Hz, J=1.7 Hz, 1H), 7.34 (d, J=8.8 Hz, 1H), 7.58 (t, J=2.3 Hz, 1H), 8.79 (s, 2H), 9.71 (s, 1H), 10.55 (s, 1H). MS (ES+): m/z=455 (M+H)⁺.

### Example 100

### N-(2-(3-(2-(Pyrrolidin-1-yl)ethoxy)phenylamino)pyrimidin-5-yl)-2,6-dimethylbenzamide (XLIII)

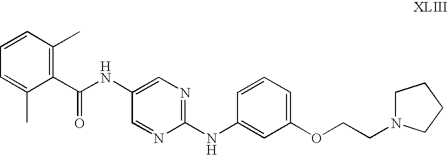

The title product was prepared by an method analogous to that described for compound XXXIX described in Example 94, except using compound 56 described in Example 98 (132 mg, 0.44 mmol) to give the title compound (16 mg, 8%) as a yellow solid. ¹H NMR (DMSO-d₆): δ 1.88-191 (m, 2H), 1.99-2.03 (m, 2H), 2.29 (s, 6H), 3.08-3.13 (m, 2H), 3.56-3.59 (m, 4H), 4.33 (t, J=4.8 Hz, 2H), 6.58 (dd, J=8.3, Hz, J=2.4 Hz, 1H), 7.12 (d, J=7.7 Hz, 2H), 7.20 (t, J=8.1, 1H), 7.25 (t, J=7.6, 11H), 7.31 (dd, J=8.0 Hz, J=1.6 Hz, 1H), 7.59 (t, J=2.2, 1H), 8.81 (s, 2H), 9.71 (s, 1H), 10.48 (s, 1H), 10.80 (br, 1H). MS (ES+): m/z=433 (M+H)⁺.

### Example 101

### N-(2-(3-(2-(Pyrrolidin-1-yl)ethoxy)phenylamino)pyrimidin-5-yl)-2,6-dichlorobenzamide (XLIV)

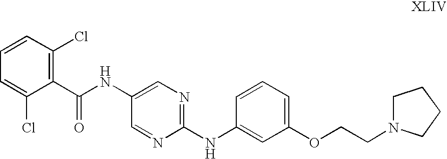

The title product was prepared by an method analogous to that of compound XXXIX described in Example 94, except compound 56 described in Example 98 (126 mg, 0.42 mmol) and 2,6-dichlorobenzoyl chloride were used to give the title compound (30 mg, 14%) as a yellow solid. ¹H NMR (DMSO-d₆): δ 1.88-191 (m, 2H), 1.99-2.03 (m, 2H), 3.08-3.13 (m, 2H), 3.56-3.59 (m, 4H), 4.33 (t, J=4.9 Hz, 2H), 6.60 (dd, J=8.0, Hz, J=2.4 Hz, 1H), 7.21 (t, J=8.1, 1H), 7.32 (dd, J=7.6 Hz, J=1.0 Hz, 1H), 7.53 (t, J=7.5, 11H), 7.58 (t, J=2.1 Hz, 1H), 7.61 (d, J=8.2 Hz, 2H), 8.78 (s, 2H), 9.77 (s, 1H), 10.94 (s, 1H), 10.68 (br, 1H). MS (ES+): m/z=474 (M+H)⁺.

### Example 102

### 3-(2-(3-(2-(Pyrrolidin-1-yl)ethoxy)phenylamino)pyrimidin-5-ylamino)-4-chlorophenol (XLV)

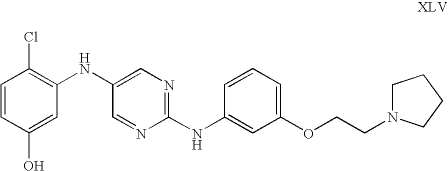

To a solution of compound 56 described in Example 98 (100 mg, 0.33 mmol) in anhydrous 1,4-dioxane (30 mL) was added 2-bromo-1-chloro-4-methoxybenzene (82 mg, 0.36 mmol), Cs₂CO₃ (436 mg, 1.33 mmol), Pd₂(dba)₃ (31 mg, 0.03 mmol), and Xantphos (58 mg, 0.10 mmol). The suspension was heated under reflux for 4 h under Ar. The solid was filtered off and washed with EtOAc. The filtrate was washed with brine (1×100 mL) and the aqueous was extracted with EtOAc (3×50 mL). Combined organic solution was dried (Na₂SO₄) and concentrated until 10 ml remain solution before adding hexane (100 mL). The mixture was sonicated for 2 min. The solid was collected by filtration and washed with hexane. The solvent was removed in vacuo and the crude material was further purified by flash column (SiO₂/CH₂Cl₂, then CH₂Cl₂:MeOH:NH₃.H₂O=100:10:1). The obtained yellow solid was dissolved in anhydrous CH₂Cl₂ (10 mL). The 1.0 M BBr₃ in CH₂Cl₂ (1.0 mL, 1.0 mmol) was added. The reaction mixture was stirred for 4 h at room temperature. The saturated NaHCO₃ (20 ml) was added and sonicated. The organic layer was separated and aqueous was extracted with CH₂Cl₂ (3×20 mL). The combined organic solution was dried (Na₂SO₄). The solvent was removed in vacuo. The crude product was purified by using HPLC. The HPLC fractions containing product were combined and neutralized with saturated NaHCO₃ (50 mL). The free base was extracted with EtOAc (2×50 mL). The combined organic layer was dried (Na₂SO₄). The solvent was removed in vacuo. The free base was dissolved in MeOH (2 mL) and the 2.0 M solution of HCl (0.2 mL, 0.4 mmol) in Et₂O was added. The solution was stirred for 5 min at room temperature before removing solvent. The residue was dissolved in MeOH (1 mL) and anhydrous Et₂O (20 mL) was added. The solid was collected by centrifuging and the HCl salt of the title compound (28 mg, 18%) was afforded as a yellow solid.

¹H NMR (500 MHz, DMSO-d₆): δ 1.88-191 (m, 2H), 1.99-2.04 (m, 2H), 3.08-3.13 (m, 2H), 3.54-3.58 (m, 4H), 4.31 (t, J=4.8 Hz, 2H), 6.20 (dd, J=8.5 Hz, J=2.6 Hz, 1H), 6.26 (d, J=2.8 Hz, 2H), 6.59 (dd, J=7.9 Hz, J=2.4 Hz, 1H), 7.13 (d, J=8.6 Hz, 1H), 7.21 (t, J=8.2 Hz, 1H), 7.34 (m, 2H), 7.57 (t, J=2.2 Hz, 1H), 8.38 (s, 2H), 9.65 (s, 1H). MS (ES+): m/z=426 (M+H)⁺.

### Example 103

### tert-butyl 4-(4-(5-aminopyrimidin-2-ylamino)phenylsulfonyl)piperidine-1-carboxylate (57)

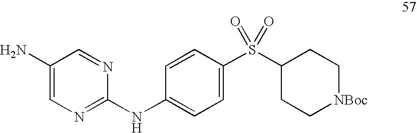

To a solution of the 2-amino-5-nitropyrimidine (200 mg, 1.4 mmol) in anhydrous 1,4-dioxane (20 mL) was added tert-butyl 4-(4-bromophenylsulfonyl)piperidine-1-carboxylate (404 mg, 1.0 mmol), Cs₂CO₃ (1.30 g, 4.0 mmol), Pd₂(dba)₃ (92 mg, 0.10 mmol), and Xantphos (173 mg, 0.30 mmol). The suspension was heated under reflux for 2 h under Ar. The solid was filtered off and washed with EtOAc. The filtrate was washed with brine (1×100 mL) and the aqueous was extracted with EtOAc (3×50 mL). Combined organic solution was dried (Na₂SO₄) and concentrated until 10 ml remain solution before adding hexane (100 mL). The mixture was sonicated for 2 min. The solid was collected by filtration and washed with hexane. The crude material was further purified by flash column (SiO₂/CH₂Cl₂, then CH₂Cl₂:MeOH:NH₃.H₂O=100:10:1). The obtained yellow solid was dissolved in MeOH (200 mL) and bubbled with Ar for 2 min before adding Ranney Ni. The mixture was hydrogenated for 1 h at room temperature. The catalyst was filtered off and washed with MeOH. The filtrate was concentrated in vacuo. The desired product was obtained as a yellow solid.

### Example 104

### N-(2-(4-(Piperidin-4-ylsulfonyl)phenylamino)pyrimidin-5-yl)-3-hydroxybenzamide (XLVI)

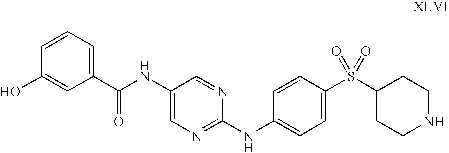

To a solution of 3-methoxybenzoic acid (183 mg, 1.20 mmol) in anhydrous CH₂Cl₂ (20 mL) was added 2-chloro-4,6-diimethyoxy-1,3,5-triazine (CDMT, 211 mg, 1.20 mmol), and 4-methylmopholine (NMM, 0.44 mL, 4.0 mmol). The mixture was stirred for 0.5 h at room temperature followed by adding compound 57 described in Example 103 (1.0 mmol). The mixture was stirred overnight at room temperature. The saturated NaHCO₃ (40 mL) was added and the mixture was stirred for 5 min. The organic layer was separated and aqueous was extracted with CH₂Cl₂ (3×20 mL). The combined organic solution was dried (Na₂SO₄). The solvent was removed in vacuo. The residue was dissolved in anhydrous CH₂Cl₂ (10 mL) and the 1.0 M BBr₃ in CH₂Cl₂ (3.0 mL, 3.0 mmol) was added. The reaction mixture was stirred for 4 h at room temperature. The saturated NaHCO₃ (20 ml) was added and sonicated. The organic layer was separated and aqueous was extracted with CH₂Cl₂ (3×20 mL). The combined organic solution was dried (Na₂SO₄). The solvent was removed in vacuo. The crude product was purified by using HPLC. The HPLC fractions containing product were combined and neutralized with saturated NaHCO₃ (50 mL). The free base was extracted with EtOAc (2×50 mL). The combined organic layer was dried (Na₂SO₄). The solvent was removed in vacuo. The free base was dissolved in MeOH (2 mL) and the 2.0 M solution of HCl (0.5 mL, 1.0 mmol) in Et₂O was added. The solution was stirred for 5 min at room temperature before removing solvent. The residue was dissolved in MeOH (1 mL) and anhydrous Et₂O (20 mL) was added. The solid was collected by centrifuging and the HCl salt of the title compound (70 mg, 14%) was afforded as a yellow solid.

¹H NMR (500 MHz, DMSO-d₆): δ 1.64-173 (m, 2H), 2.02 (d, J=13.1 Hz, 2H), 2.82-2.89 (m, 2H), 3.44-3.50 (m, 4H), 7.01 (dd, J=8.0 Hz, J=2.4 Hz, 1H), 7.33-7.37 (m, 2H), 7.42 (d, J=7.7 Hz, 1H), 7.73 (d, J=9.0 Hz, 2H), 8.05 (d, J=9.0 Hz, 2H), 8.40-8.44 (m, 1H), 8.93 (s, 2H), 9.84 (s, 1H), 10.31 (s, 1H), 10.40 (s, 1H). MS (ES+): m/z=454 (M+H)⁺.

### Example 105

### General Procedure for the Reduction of Nitro-Compound (Method A)

A solution of nitro-compound (1.0 mol equiv) in MeOH (0.05-1.0 M) can be flushed with argon and then Pd/C (10% by wt) added. The mixture can be evacuated and then refilled with hydrogen and stirred at room temperature for 2-4 h. The heterogeneous reaction mixture can be filtered through a pad of Celite, washed with MeOH and concentrated in vacuo to furnish the corresponding amino-compound. The crude amino-compound can be used in the next step without purification.

### Example 106

### General Procedure for the Amide Bond Formation (Method B)

To a solution of an amino-compound (1.0 mol equiv) and a carboxylic acid (1.2 mol equiv) in dry DMF (0.05-0.2 M) can be added HBTU (1.5 mol equiv) and HOBt (1.3 mol equiv) followed by DIPEA (3.0 mol equiv). The reaction mixture can be stirred at room temperature for 16 h and then diluted with EtOAc. The organic layer can be washed with water, brine, dried over MgSO₄ and filtered. The filtrate can be concentrated in vacuo and the crude product purified as described below.

### Example 107

### General Procedure for the Deprotection of Methoxy Precursor with BBr₃ (Method C)

To a solution or suspension of methoxy precursor (1.0 mol equiv) in DCM (0.01-0.03 M) at room temperature can be added BBr₃ (5-10 mol equiv) and the mixture stirred at room temperature for 4-12 h. The reaction can be quenched with saturated NaHCO₃ solution until the pH is about 7 and the resulting solid filtered. The filtered solid can be washed with large amount of water and ether. The solid obtained can be tested for enzyme activity directly or further purified if required.

### Example 108

### 4-(4-Bromo-benzenesulfonyl)-piperazine-1-carboxylic acid tert-butyl ester (58)

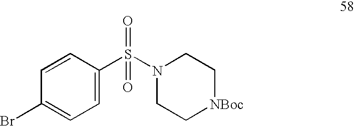

To a solution of 4-bromo-benzenesulfonyl chloride (1.0 g, 3.9 mmol) and piperazine-1-carboxylic acid tert-butyl ester (1.0 g, 5.4 mmol) in dry DCM (15 mL) was added triethylamine (1.6 mL, 11 mmol). The reaction mixture was stirred at room temperature for 2.5 h and then diluted with EtOAc. The organic layer was washed with saturated NaHCO₃, brine, dried over MgSO₄ and filtered. The filtrate was concentrated to afford the title compound, which was used in the next step without purification.

### Example 109

### 4-[4-(5-Nitro-pyrimidin-2-ylamino)-benzenesulfonyl]-piperazine-1-carboxylic acid tert-butyl ester (59)

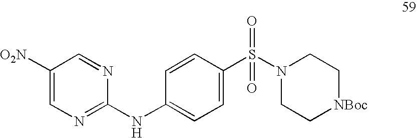

A mixture of 5-nitro-pyrimidin-2-ylamine (0.25 g, 1.8 mmol), compound 58 described in Example 108 (1.0 g, 2.5 mmol), Pd(OAc)₂ (20 mg, 0.09 mmol), Xantphos (0.1 g, 0.17 mmol) and potassium tert-butoxide (0.40 g, 3.6 mmol) were suspended in dioxane (15 mL) and heated at reflux under the argon atmosphere for 16 h. The mixture was allowed to cool to room temperature, filtered and washed with DCM. The filtrate was concentrated and the residue triturated in DCM-Et₂O (1:5 v/v) and the solid filtered. The solid was washed with Et₂O to afford the title compound (0.25 g, 30%) as an orange solid. The crude title compound was used in the next step without purification. MS (ES+): m/z 365 (M+H-Boc)⁺.

### Example 110

### 4-[4-(5-Amino-pyrimidin-2-ylamino)-benzenesulfonyl]-piperazine-1-carboxylic acid tert-butyl ester (60)

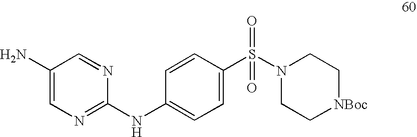

The title compound was prepared from compound 59 described in Example 109 (0.25 g, 0.54 mmol) according to method A described in Example 105 and used in the next step without purification. MS (ES+): m/z 335 (M+H-Boc)⁺.

### Example 111

### 4-{4-[5-(2-Chloro-5-methoxy-benzoylamino)-pyrimidin-2-ylamino]-benzenesulfonyl}-piperazine-1-carboxylic acid tert-butyl ester (61)

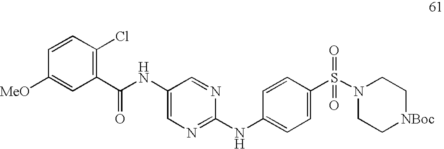

The title compound was prepared from compound 60 described in Example 110 (0.20 g, 0.45 mmol) and 2-chloro-5-methoxy-benzoic acid according to method B described in Example 106 and the crude product purified by flash chromatography on silica gel (40% EtOAc/hexane) to afford 4 (0.1 g, 33%) as a white solid. MS (ES+): m/z 503 (M+H-Boc)⁺.

### Example 112

### 2-Chloro-5-hydroxy-N-{2-[4-(piperazine-1-sulfonyl)-phenylamino]-Pyrimidin-5-yl}-benzamide (XLVII)

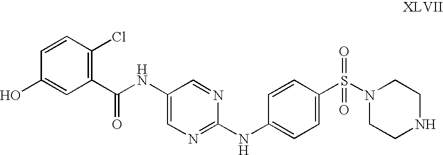

The title compound was prepared from compound 61 described in Example 111 by method C described in Example 107 and the Boc-protecting group simultaneously removed. Pale yellow solid (50 mg, 67% yield). ¹H NMR (DMSO-d₆): δ 2.65-2.85 (m, 8H), 6.91 (dd, J=8.8 Hz, J=2.9 Hz, 1H), 6.97 (d, J=2.9 Hz, 1H), 7.35 (d, J=8.8 Hz, 2H), 7.63 (d, J=8.9 Hz, 2H), 7.99 (d, J=8.9 Hz, 2H), 8.87 (s, 2H), 10.06 (bs, 1H), 10.25 (s, 1H), 10.63 (s, 1H). MS (ES+): m/z 489 (M+H)⁺.

### Example 113

### 2-Chloro-N-(2-[6-{4-(2-hydroxy-ethyl)-piperazine-1-carbonyl]-pyridin-3-ylamino}-pyrimidin-5-yl)-5-methoxy-benzamide (62)

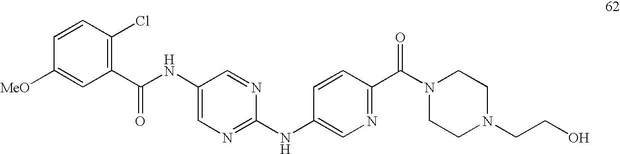

The title compound was prepared from compound 5 described in Example 7 (0.25 g, 0.73 mmol) and 2-chloro-5-methoxy-benzoic acid according to method B described in Example 106 and the crude product (0.25 g) used in the next step without purification. MS (ES+): m/z 512 (M+H)⁺.

### Example 114

### 2-Chloro-5-hydroxy-N-(2-{6-[4-(2-hydroxy-ethyl)-piperazine-1-carbonyl]-pyridin-3-ylamino}-pyrimidin-5-yl)-benzamide (XLVIII)

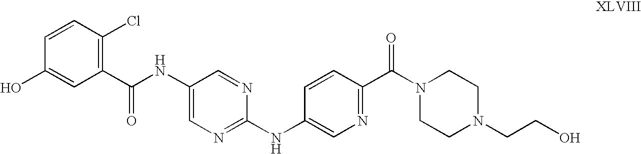

The title compound was prepared from compound 62 described in Example 113 (0.10 g, 0.20 mmol) by method C described in Example 107 and the crude product purified by HPLC to afford the title compound as an off white solid (13 mg, 11% yield). ¹H NMR (DMSO-d₆): δ 3.05-3.30 (m, 4H), 3.40-3.65 (m, 4H), 3.65-3.75 (m, 2H), 4.40-4.65 (m, 2H), 5.41 (bs, 1H), 6.91 (dd, J=8.8 Hz, J=2.9 Hz, 1H), 6.97 (d, J=2.9 Hz, 1H), 7.35 (d, J=8.7 Hz, 1H), 7.70 (d, J=8.7 Hz, 1H), 8.38 (dd, J=8.8 Hz, J=2.6 Hz, 1H), 8.87 (s, 2H), 8.91 (d, J=2.5 Hz, 1H), 9.75 (bs, 1H), 10.08 (s, 1H), 10.23 (s, 1H), 10.63 (s, 1H) MS (ES+): m/z 498 (M+H)⁺.

### Example 115

### 2-[4-(5-Bromo-pyridin-2-yl)-piperazin-1-yl]-ethanol (63)

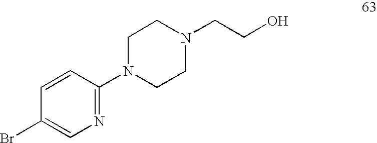

A mixture of 5-bromo-2-iodo-pyridine (5.0 g, 18 mmol) and 2-piperazin-1-yl-ethanol (5.0 g, 39 mmol) in acetonitrile (40 mL) was heated at reflux for 1 d. The mixture was allowed to cooled to room temperature, poured into water and extracted with EtOAc. The organic layer was washed with water, brine, and dried over MgSO₄ and filtered. The filtrate was concentrated and the residue purified by flash chromatography on silica gel (5% MeOH/DCM to 10% MeOH/DCM) to afford the title compound (1.3 g, 26%) as a white solid.

### Example 116

### 2-{4-[5-(5-Nitro-pyrimidin-2-ylamino)-pyridin-2-yl]-piperazin-1-yl}-ethanol (64)

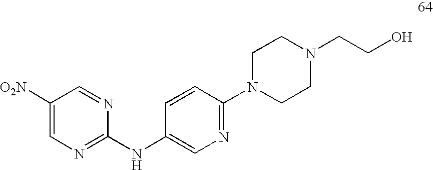

A mixture of 5-nitro-pyrimidin-2-ylamine (0.30 g, 2.1 mmol), compound 63 described in Example 115 (2.5 g, 2.8 mmol), Pd₂(dba)₃ (0.10 g, 0.11 mmol), Xantphos (0.13 g, 0.22 mmol) and cesium carbonate (1.4 g, 4.3 mmol) were suspended in dioxane (30 mL) and heated at reflux under the argon atmosphere for 18 h. The mixture was allowed to cool to room temperature, filtered and washed with DCM. The filtrate was concentrated and the crude product purified by flash chromatography on silica gel (10% MeOH/DCM to 15% MeOH/DCM) to afford the final title product (0.30 g, 41%) as an off-white solid. MS (ES+): m/z 346 (M+H)⁺.

### Example 117

### 2-{4-[5-(5-Amino-pyrimidin-2-ylamino)-pyridin-2-yl]-piperazin-1-yl}-ethanol (65)

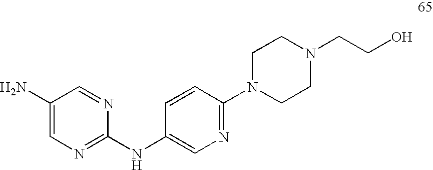

The title compound was prepared from compound 64 described in Example 116 (0.30 g, 0.87 mmol) according to method A described in Example 105 and used in the next step without purification. MS (ES+): m/z 316 (M+H)⁺.

### Example 118

### 2,6-Dichloro-N-(2-{6-[4-(2-hydroxy-ethyl)-piperazin-1-yl]-pyridin-3-ylamino}-pyrimidin-5-yl)-benzamide (XLIX)

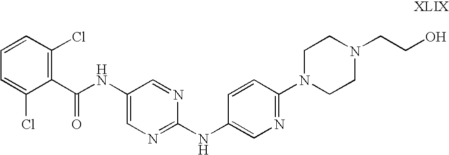

To a solution of compound 65 described in Example 117 (0.25 g, 0.80 mmol) and 2,6-dichloro-benzoyl chloride (0.40 g, 1.9 mmol) in THF (20 mL) was added triethylamine (0.30 mL, 2.2 mmol). The mixture was heated at reflux for 17 h. The mixture was allowed to cool to room temperature and most of the THF removed. The resulting residue was redissolved in EtOAc, washed with saturated NaHCO₃, brine, dried over MgSO₄ and filtered. The filtrate was concentrated and the residue purified by flash chromatography on silica gel (5% MeOH/DCM to 20% MeOH/DCM) to afford the free base compound and furnished final title product (30 mg, 8% overall) which changed to a pale yellow gel on exposure to air.

¹H NMR (DMSO-d₆): δ 3.15-3.30 (m, 4H), 3.50-3.70 (m, 4H), 3.83 (t, J=5.2 Hz, 2H), 4.35-4.45 (m, 2H), 7.30-7.40 (m, 1H), 7.52 (d, J=7.3 Hz, 1H), 7.54 (d, J=7.3 Hz, 1H), 7.60 (d, J=8.7 Hz, 1H), 8.19 (dd, J=9.4 Hz, J=2.3 Hz, 1H), 8.66 (d, J=2.6 Hz, 1H), 8.80 (s, 2H), 9.98 (bs, 1H), 10.96 (bs, 1H), 11.00 (s, 1H). MS (ES+): m/z 448 (M+H)⁺.

### Example 119

### 4-(4-Bromo-benzoyl)-piperazine-1-carboxylic acid tert-butyl ester (66)

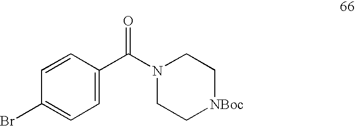

To a solution of 4-bromo-benzoyl chloride (1.0 g, 4.5 mmol) and piperazine-1-carboxylic acid tert-butyl ester (1.1 g, 5.9 mmol) in dry DCM (15 mL) was added triethylamine (1.5 mL, 11 mmol). The reaction mixture was stirred at room temperature for 12 h and then diluted with EtOAc. The organic layer was washed with saturated NaHCO₃, brine, dried over MgSO₄ and filtered. The filtrate was concentrated and the resulting solid was triturated in hexane-Et₂O (10:1 v/v) and the solid filtered. The solid was washed with Et₂O to afford the title compound (1.6 g, 95%) as a white solid.

### Example 120

### 4-[4-(5-Nitro-pyrimidin-2-ylamino)-benzoyl]-piperazine-1-carboxylic acid tert-butyl ester (67)

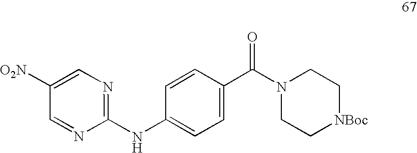

A mixture of 5-nitro-pyrimidin-2-ylamine (0.90 g, 6.4 mmol), compound 66 described in Example 119 (3.1 g, 8.4 mmol), Pd(OAc)₂ (0.10 g, 0.44 mmol), Xantphos (0.52 g, 0.89 mmol) and potassium tert-butoxide (1.5 g, 13 mmol) were suspended in dioxane (15 mL) and heated at reflux under the argon atmosphere for 5 h. The mixture was allowed to cool to room temperature, filtered and washed with DCM. The filtrate was concentrated and the residue triturated in Et₂O and the title compound obtained as a yellow solid after filtration (0.70 g). The filtrate was concentrated again and the residue purified by flash chromatography on silica gel (40% EtOAc/hexane) to afford the additional product (0.70 g, 51% overall). ¹H NMR (DMSO-d₆): 62.35-2.45 (m, 4H), 3.45-3.55 (m, 4H), 3.60-3.70 (m, 2H), 4.43 (t, J=5.4 Hz, 2H), 7.64 (d, J=8.5 Hz, 1H), 8.31 (dd, J=8.7 Hz, J=2.5 Hz, 1H), 8.93 (d, J=2.2 Hz, 1H), 9.30 (s, 2H), 11.17 (s, 1H). MS (ES+): m/z 329 (M+H-Boc)⁺.

### Example 121

### 4-[4-(5-Amino-pyrimidin-2-lamino)-benzoyl]-piperazine-1-carboxylic acid tert-butyl ester (68)

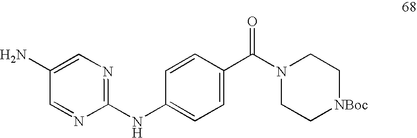

The title compound was prepared from compound 67 described in Example 120 (1.3 g, 3.0 mmol) according to method A described in Example 105 and the residue triturated in Et₂O and the title compound obtained as a yellow solid after filtration (0.50 g). The filtrate was concentrated and the residue purified by flash chromatography on silica gel (5% MeOH/DCM) to afford the additional product (0.15 g, 54% overall). MS (ES+): m/z 399 (M+H)⁺.

### Example 122

### 4-{4-[5-(2,6-Dimethyl-benzoylamino)-pyrimidin-2-ylamino]-benzoyl}-piperazine-1-carboxylic acid tert-butyl ester (69)

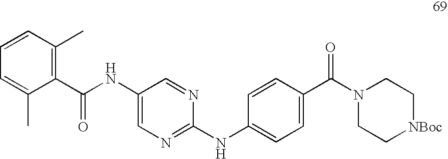

To a solution of compound 68 described in Example 121 (0.20 g, 0.50 mmol) and 2,6-dimethyl-benzoyl chloride (0.20 g, 1.2 mmol) in THF (15 mL) was added triethylamine (0.20 mL, 1.4 mmol). The mixture was heated at reflux for 19 h. The mixture was allowed to cool to room temperature and most of the THF removed. The resulting residue was redissolved in EtOAc, washed with saturated NaHCO₃, brine, dried over MgSO₄ and filtered. The filtrate was concentrated and the residue purified by flash chromatography on silica gel (5% MeOH/DCM) to afford the title compound as a pale yellow solid (60 mg, 23%).

### Example 123

### 2,6-Dimethyl-N-{2-[4-(piperazine-1-carbonyl)-phenylamino]-pyrimidin-5-yl}-benzamide (L)

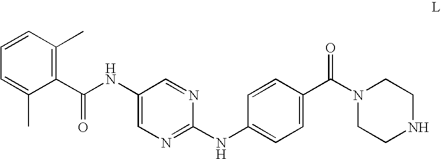

A solution of compound 69 described in Example 122 in 30% TFA/DCM (6 mL) was stirred at room temperature for 30 min. The solvent was removed and the residue purified by HPLC. The combined fractions were poured into saturated NaHCO₃ solution and extracted with EtOAc. The organic layers were combined and washed with brine, dried over MgSO₄ and filtered. The filtrate was concentrated and the residue was triturated in DCM-Et₂O (1:5, v/v) and the title compound obtained as a white solid after filtration (10 mg, 22%).

¹H NMR (DMSO-d₆): δ 2.30 (s, 6H), 2.65-2.75 (m, 4H), 3.45-3.50 (m, 4H), 7.13 (d, J=7.6 Hz, 2H), 7.25 (t, J=7.7 Hz, 1H), 7.32 (d, J=7.0 Hz, 2H), 7.81 (d, J=7.0 Hz, 2H), 8.83 (s, 2H), 9.90 (s, 1H), 10.48 (s, 1H). MS (ES+): m/z 431 (M+H)⁺.

### Example 124

### 4-{4-[5-(2-Chloro-5-methoxy-benzoylamino)-pyrimidin-2-ylamino]-benzoyl}-piperazine-1-carboxylic acid tert-butyl ester (70)

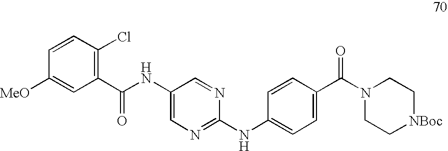

The title compound was prepared from compound 68 described in Example 121 (0.20 g, 0.50 mmol) and 2-chloro-5-methoxy-benzoic acid according to method B described in Example 106 and the crude product purified by flash chromatography on silica gel (60% EtOAc/hexane) to afford the title compound (0.15 g, 53%) as a pale yellow solid. MS (ES+): m/z 567 (M+H)⁺.

### Example 125

### 2-Chloro-5-hydroxy-N-{2-[4-(piperazine-1-carbonyl)-phenylamino]-Pyrimidin-5-yl}-benzamide (LI)

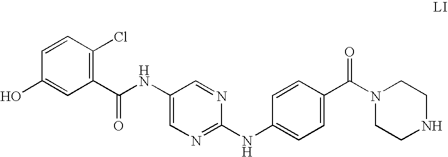

The title compound was prepared from compound 70 described in Example 124 by method C described in Example 107 and the Boc-protecting group simultaneously removed. The crude product was purified by HPLC and the combined fractions concentrated in high vacuum to afford the title compound as an off white solid (12 mg, 9%).

¹H NMR (DMSO-d₆): δ 3.10-3.20 (m, 4H), 3.60-3.75 (m, 4H), 6.91 (dd, J=8.8 Hz, J=3.0 Hz, 1H), 6.96 (d, J=2.9 Hz, 1H), 7.35 (d, J=8.7 Hz, 1H), 7.42 (d, J=8.8 Hz, 2H), 7.85 (d, J=8.8 Hz, 2H), 8.82 (s, 2H), 8.86 (bs, 1H), 9.97 (s, 1H), 10.08 (s, 1H), 10.57 (s, 1H). MS (ES+): m/z 453 (M+H)⁺.

### Example 126

### N′-(2,6-Dichloro-benzyl)-N-[3-(2-pyrrolidin-1-yl-ethoxy)-phenyl]-pyrimidine-2,5-diamine (LII)

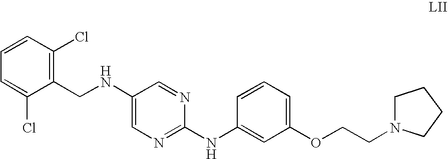

A solution of compound 56 described in Example 98 (0.10 g, 0.33 mmol), 2,6-dichlorobenzyl bromide (0.10 g, 0.42 mmol) and cesium carbonate (0.25 g, 0.77 mmol) in dioxane/DMF (18 mL, 5/1 v/v) was stirred at 105° C. for 1 d. The reaction mixture was cooled to room temperature and then poured into water. The aqueous layer was extracted with EtOAc and the combined organic layers washed with brine, dried over Na₂SO₄ and filtered. The filtrate was concentrated and the residue purified by HPLC. The corrected fractions were poured into saturated NaHCO₃ and extracted with EtOAc. The combined organic layers were washed with brine, dried over Na₂SO₄ and filtered. The filtrate was concentrated to afford the free base compound. The free base compound furnished the title compound as a yellow solid (20 mg, 12% overall).

¹H NMR (DMSO-d₆): δ 1.85-1.95 (m, 2H), 1.95-2.05 (m, 2H), 3.05-3.15 (m, 2H), 3.50-3.60 (m, 4H), 4.29 (t, J=4.9 Hz, 2H), 4.41 (s, 2H), 6.49 (dd, J=7.7 Hz, J=2.4 Hz, 1H), 7.15 (t, J=8.1 Hz, 1H), 7.25 (dd, J=8.3 Hz, J=1.6 Hz, 1H), 7.40 (t, J=7.8 Hz, 1H), 7.50 (t, J=2.2 Hz, 1H), 7.53 (d, J=8.1 Hz, 2H), 8.12 (s, 2H), 9.18 (bs, 1H), 10.41 (bs, 1H). MS (ES+): m/z 458 (M+H)⁺.

### Example 127

### 2-[4-(6-Chloro-2-methyl-pyrimidin-4-yl)-piperazin-1-yl]-ethanol (71)

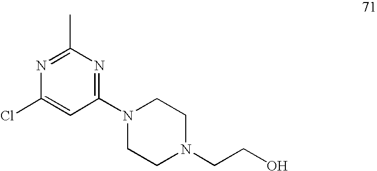

To a solution of 4,6-dichloro-2-methyl-pyrimidine (5.0 g, 31 mmol) and 2-piperazin-1-yl-ethanol (2.7 g, 21 mmol) in dioxane (25 mL) was added DIPEA (3.0 mL, 17 mmol). The mixture was heated at reflux for 16 h. The mixture was allowed to cool to room temperature and poured into water. The resulting aqueous layer was extracted with EtOAc and the combined organic layers washed with brine, dried over Na₂SO₄ and filtered. The filtrate was concentrated and the residue purified by flash chromatography on silica gel (5-10% MeOH/DCM) to afford the title compound as a brown liquid (2.1 g, 39%). MS (ES+): m/z 257 (M+H)⁺.

### Example 128

### 2-{4-[2-Methyl-6-(5-nitro-pyrimidin-2-ylamino)-pyrimidin-4-yl]-piperazin-1-yl}-ethanol (72)

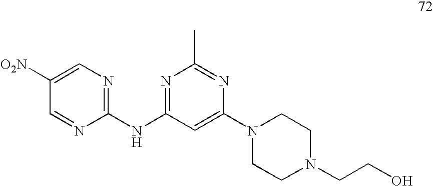

A mixture of 5-nitro-pyrimidin-2-ylamine (0.45 g, 3.2 mmol), compound 71 described in Example 127 (1.0 g, 3.9 mmol), Pd(OAc)₂ (50 mg, 0.22 mmol), Xantphos (0.26 g, 0.45 mmol) and potassium tert-butoxide (0.72 g, 6.4 mmol) were suspended in dioxane (15 mL) and heated at reflux under the argon atmosphere for 15 h. The mixture was allowed to cool to room temperature, filtered and washed with DCM. The filtered solid was washed with water and DCM to afford the title compound (0.60 g, 52%), which was used in the next step without further purification. MS (ES+): m/z 361 (M+H)⁺.

### Example 129

### 2-{4-[6-(5-Amino-pyrimidin-2-ylamino)-2-methyl-pyrimidin-4-yl]-piperazin-1-yl}-ethanol (73)

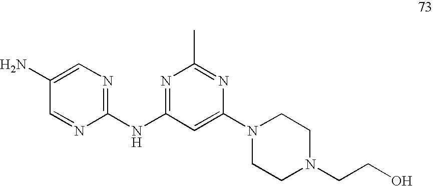

The title compound was prepared from compound 72 described in Example 128 (0.60 g, 1.7 mmol) according to method A described in Example 105 and the residue triturated in Et₂O and the title compound obtained as a pale yellow solid after filtration (0.47 g, 85%). MS (ES+): m/z 331 (M+H)⁺.

### Example 130

### 2,6-Dichloro-N-(2-{6-[4-(2-hydroxy-ethyl)-piperazin-1-yl]-2-methyl-pyrimidin-4-ylamino}-pyrimidin-5-yl)-benzamide (LIII)

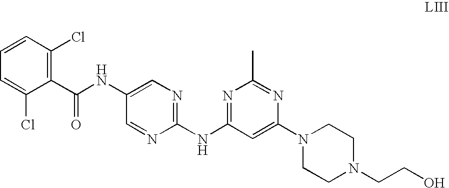

A solution of compound 73 described in Example 129 (0.10 g, 0.30 mmol), 2,6-dichloro-benzoyl chloride (90 mg, 0.43 mmol) and cesium carbonate (0.20 g, 0.61 mmol) in dioxane/DMF (11 mL, 10/1, v/v) was heated at 105° C. for 16 h. The mixture was allowed to cool to room temperature and poured into water. The aqueous layer was extracted with EtOAc and the combined organic layers washed with brine, dried over Na₂SO₄ and filtered. The filtrate was concentrated and the residue purified by flash HPLC. The corrected fractions were poured into saturated NaHCO₃ and extracted with EtOAc. The combined organic layers were washed with brine, dried over Na₂SO₄ and filtered. The filtrate was concentrated to afford the free base compound, which furnished the title compound as a white solid (30 mg, 19% overall).

¹H NMR (DMSO-d₆): δ 2.55 (s, 3H), 3.15-3.25 (m, 4H), 3.55-3.70 (m, 6H), 3.81 (t, J=4.9 Hz, 2H), 7.28 (bs, 1H), 7.50-7.65 (m, 3H), 9.05 (s, 2H), 10.90 (bs, 1H), 11.15 (bs, 1H), 11.34 (s, 1H). MS (ES+): m/z 504 (M+H)⁺.

### Example 131

### 6-Chloro-2-fluoro-3-hydroxy-benzoic acid (74)

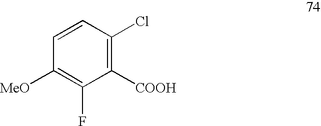

To a solution of 4-chloro-2-fluoro-1-methoxy-benzene (2.0 g, 12.5 mmol) in THF (20 mL) at −78° C. under the argon atmosphere was added n-butyl lithium (2.5 M in hexane; 7.5 mL, 19 mmol) slowly. The mixture was stirred at the same temperataure for 30 min and couple pieces of dry-ice pellets added. The temperature was raised to room temperature over 4 h. The reaction was quenched with water carefully and then diluted with NaOH solution until the pH ˜10. The mixture was extracted with ethyl acetate and the organic separated. The organic layer was acidified with conc. HCl until pH ˜2 and the resulting white solid filtered. The solid (2.7 g, 98%) was washed with water and used in the next step without further purification. ¹H NMR (500 MHz, DMSO-d₆): δ 7.27 (t, J=9.0 Hz, 1H), 7.32 (dd, J=8.9 Hz, J=1.4 Hz, 1H), 3.70 (s, 3H).

### Example 132

### 6-Chloro-2-fluoro-3-methoxy-N-{2-[4-(3-pyrrolidin-1-yl-propane-1-sulfonyl)-phenylamino]-pyrimidin-5-yl}-benzamide (75)

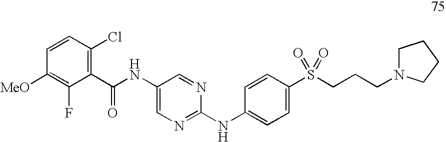

The title compound was prepared from compound 52 described in Example 83 (0.30 g, 0.84 mmol) and 74 (1.9 g, 0.93 mmol) according to method B described in Example 106 and the crude product purified by flash chromatography on silica gel (20% MeOH/DCM to 18% MeOH and 2% TEA/DCM) to afford the title compound (0.37 g, 81%) as a yellow foam. MS (ES+): m/z 548 (M+H)⁺.

### Example 133

### 6-Chloro-2-fluoro-3-hydroxy-N-{2-[4-(3-pyrrolidin-1-yl-propane-1-sulfonyl)-phenylamino]-pyrimidin-5-yl}-benzamide (LIV)

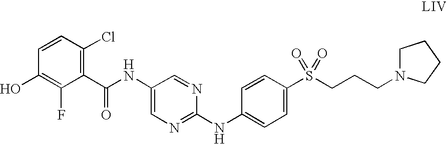

The title compound was prepared from compound 75 described in Example 132 (0.16 g, 0.33 mmol) by method C described in Example 107 and the crude product purified by HPLC to afford the title compound as an orange solid (TFA salt; 30 mg, 16%).

¹H NMR (500 MHz, DMSO-d₆): δ 1.65-1.75 (m, 6H), 2.40-2.60 (m, 4H), 3.20-3.30 (m, 2H), 3.30-3.40 (m, 2H), 7.09 (t, J=9.1 Hz, 1H), 7.23 (dd, J=8.9 Hz, J=1.4 Hz, 1H), 7.78 (d, J=9.0 Hz, 2H), 8.01 (d, J=9.0 Hz, 2H), 8.87 (s, 2H), 10.32 (s, 1H), 10.99 (s, 1H). MS (ES+): m/z 534 (M+H)⁺.

### Example 134

### (3-Bromo-4-methyl-phenyl)-methanol (76)

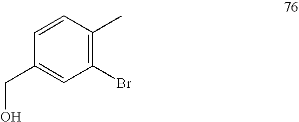

To a solution of 3-bromo-4-methyl-benzoic acid (2.0 g, 9.3 mmol) in THF (10 mL) at 0° C. under the argon atmosphere was added LiAlH₄ (1.0 M in THF; 10 mL, 10 mmol). After the addition, the temperature was raised to room temperature and the mixture stirred for 2 h. The mixture was then refluxed for additional 2 h and allowed to cool to room temperature. The reaction was quenched with 1 M HCl until the pH ˜4 and the resulting solid filtered and washed with ethyl acetate. The organic layer was separated and washed with brine. The organic layer was dried over Na₂SO₄ and filtered. The filtrate was concentrated and the crude product used in the next step without further purification.

¹H NMR (500 MHz, DMSO-d₆): δ 2.32 (s, 3H), 4.45 (d, J=5.8 Hz, 2H), 7.20 (dd, J=7.8 Hz, J=1.2 Hz, 1H), 7.29 (d, J=7.8 Hz, 1H), 7.51 (s, 1H).

### Example 135

### 5-Hydroxymethyl-2-methyl-benzoic acid (77)

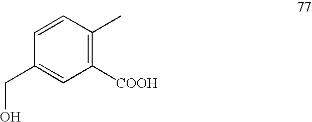

To a solution of compound 76 described in Example 134 (1.8 g, 9.0 mmol) in THF (20 mL) at −78° C. under the argon atmosphere was added slowly n-butyl lithium (2.5 M in hexane; 7.0 mL, 15 mmol). The mixture was stirred at the same temperataure for 30 min and couple pieces of dry-ice pellet added. The temperature was raised to room temperature over 4 h and the reaction quenched with 1M HCl carefully and then extracted with ethyl acetate. The mixture was extracted with ethyl acetate and the organic separated. The organic layer was washed with brine, dried over Na₂SO₄ and filtered. The filtrate was concentrated and the crude product used in the next step without further purification.

### Example 136

### 5-Hydroxymethyl-2-methyl-N-{2-[4-(3-pyrrolidin-1-yl-propane-1-sulfonyl)-phenylamino]-pyrimidin-5-yl}-benzamide (LV)

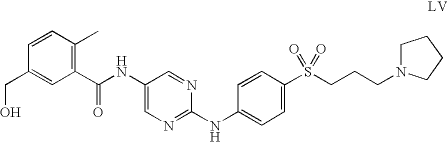

The title compound was prepared from compound 77 described in Example 135 (0.50 g, 3.0 mmol) and compound 52 described in Example 83 (0.10 g, 0.30 mmol) according to method B described in Example 106 and the crude product purified by HPLC to afford the title compound (TFA salt; 30 mg, 16%) as a brown solid.

¹H NMR (500 MHz, DMSO-d₆): δ 1.75-2.05 (m, 6H), 2.39 (s, 3H), 2.90-3.00 (m, 2H), 3.15-3.25 (m, 2H), 3.30-3.40 (m, 2H), 3.50-3.60 (m, 2H), 4.54 (s, 2H), 7.28 (d, J=7.8 Hz, 1H), 7.36 (d, J=7.9 Hz, 1H), 7.46 (s, 1H), 7.80 (d, J=8.9 Hz, 2H), 8.03 (d, J=8.9 Hz, 2H), 8.91 (s, 2H), 10.29 (s, 1H), 10.48 (s, 1H). MS (ES+): m/z 510 (M+H)⁺.

### Example 137

### 3-(4-Bromo-phenyl)-propan-1-ol (78)

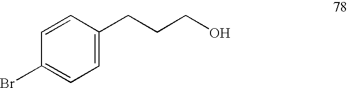

To a solution of 3-(4-bromo-phenyl)-propionic acid (4.0 g, 18 mmol) in THF (30 mL) at 0° C. under the argon atmosphere was added LiAlH₄ (1.0 M in THF; 14 mL, 14 mmol). After the addition, the ice-bath was removed and the mixture refluxed for 18 h. After cooling to room temperature, the reaction was quenched with 1 M HCl and the mixture extracted with ethyl acetate. The organic layer was separated, washed with brine, dried over Na₂SO₄ and filtered. The filtrate was concentrated and the crude product used in the next step without further purification.

### Example 138

### 1-Bromo-4-(3-bromo-propyl)-benzene (79)

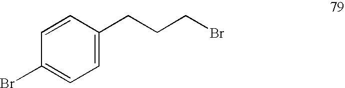

To a solution of compound 78 described in Example 137 (4.0 g, 19 mmol) in THF (30 mL) at 0° C. under the argon atmosphere was added PPh₃ (6.3 g, 24 mmol) followed by CBr₄ (8.0 g, 24 mmol). The mixture was stirred at the same temperature for 15 min and then stirred at room temperature for additional 15 h. Most of the solvent was removed and the residue purified by flash chromatography on silica gel (hexane) to afford the title compound (3.5 g, 66%) as a colorless oil.

¹H NMR (500 MHz, DMSO-d₆): δ 2.03-2.12 (m, 2H), 2.68 (t, J=7.5 Hz, 2H), 3.49 (t, J=6.5 Hz, 2H), 7.19 (d, J=8.3 Hz, 2H), 7.47 (d, J=8.3 Hz, 2H).

### Example 139

### 1-[3-(4-Bromo-phenyl)-propyl]-pyrrolidine (80)

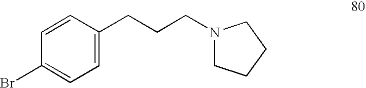

To a solution of compound 79 described in Example 138 (3.5 g, 13 mmol) in dioxane (40 mL) was added pyrrolidine (2.1 mL, 25 mmol) followed by cesium carbonate (8.2 g, 25 mmol). The mixture was stirred at room temperature for 15 h and poured into water. The mixture was extracted with ethyl acetate and the organic layer separated, washed with brine, dried over Na₂SO₄ and filtered. The filtrate was concentrated and the residue purified by flash chromatography on silica gel (10% MeOH/DCM to 25% MeOH and 2% TEA/DCM) to afford the title compound (1.8 g, 53%) as a pale orange oil.

¹H NMR (500 MHz, DMSO-d₆): δ 1.60-1.65 (m, 6H), 2.35 (t, J=7.3 Hz, 2H), 2.35-2.43 (m, 4H), 2.57 (t, J=7.7 Hz, 2H), 7.16 (d, J=8.3 Hz, 2H), 7.44 (d, J=8.4 Hz, 2H).

### Example 140

### (5-Nitro-pyrimidin-2-yl)-[4-(3-pyrrolidin-1-yl-propyl)-phenyl]-amine

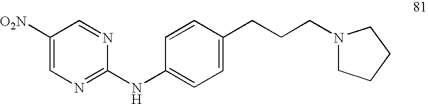

A mixture of 5-nitro-pyrimidin-2-ylamine (0.15 g, 1.1 mmol), compound 80 described in Example 139 (0.30 g, 1.1 mmol), Pd₂(dba)₂ (75 mg, 0.082 mmol), Xantphos (96 mg, 0.17 mmol) and cesium carbonate (0.69 g, 2.1 mmol) were suspended in dioxane (15 mL) and heated at reflux under the argon atmosphere for 15 h. The mixture was allowed to cool to room temperature, filtered and washed with DCM. The filtrate was concentrated and the residue purified by flash chromatography on silica gel (10% MeOH/DCM to 20% MeOH and 2% TEA/DCM) to afford the title compound as a yellow solid (0.20 g, 56%).

### Example 141

### N-[4-(3-Pyrrolidin-1-yl-propyl)-phenyl]-pyrimidine-2,5-diamine (82)

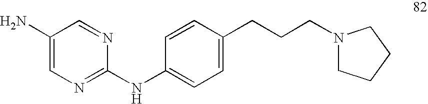

The title compound was prepared from compound 81 described in Example 140 (0.20 g, 0.61 mmol) according to method A described in Example 105 and used in the next step without further purification.

### Example 142

### 2,6-Dichloro-N-[2-[4-(3-pyrrolidin-1-yl-propyl)-phenylamino]-pyrimidin-5-yl]-benzamide (LVI)

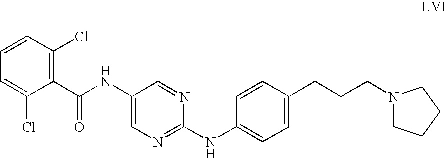

To a solution of compound 82 described in Example 141 (0.10 g, 0.33 mmol) in THF (10 mL) was added 2,6-dichloro-benzoyl chloride (0.11 g, 0.53 mmol) followed by triethylamine (0.15 mL, 1.1 mmol). The mixture was stirred at RT for 15 h and then poured into the saturated NaHCO₃ solution. The mixture was extracted with EtOAc and the combined organic layers washed with brine, dried over Na₂SO₄ and filtered. The filtrate was concentrated and purified by HPLC to afford the title compound (TFA salt; 25 mg, 13%) as a brown solid.

¹H NMR (500 MHz, DMSO-d₆): δ 1.80-2.05 (m, 6H), 2.59 (t, J=7.6 Hz, 2H), 2.95-3.05 (m, 2H), 3.05-3.15 (m, 2H), 3.50-3.60 (m, 2H), 7.14 (d, J=8.6 Hz, 2H), 7.53 (dd, J=9.0, J=7.1 Hz, 1H), 7.60 (d, J=7.3 Hz, 1H), 7.61 (d, J=8.6 Hz, 1H), 7.67 (d, J=8.5 Hz, 2H), 8.73 (s, 2H), 9.50 (bs, 1H), 9.67 (s, 1H), 10.84 (s, 1H). MS (ES+): m/z 470 (M+H)⁺.

### Example 143

### N′-(2,6-Dichloro-benzyl)-N-[4-(3-pyrrolidin-1-yl-propyl)-phenyl]-pyrimidine-2,5-diamine (LVII)

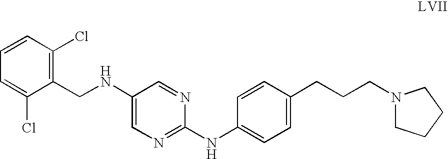

A solution of compound 82 described in Example 141 (0.30 g, 1.0 mmol), 2,6-dichlorobenzyl bromide (0.35 g, 1.5 mmol) and cesium carbonate (0.90 g, 2.8 mmol) in DMF (15 mL) was stirred at 100° C. for 8 h. The reaction mixture was cooled to room temperature and then poured into water. The aqueous layer was extracted with EtOAc and the combined organic layers washed with brine, dried over Na₂SO₄ and filtered. The filtrate was concentrated and the residue purified by HPLC to afford the title compound (TFA salt; 0.14 g, 25%) as a pale yellow solid.

¹H NMR (500 MHz, DMSO-d₆): δ 1.75-2.05 (m, 6H), 2.55 (t, J=7.6 Hz, 2H), 2.90-3.05 (m, 2H), 3.05-3.15 (m, 2H), 3.50-3.60 (m, 2H), 4.40 (s, 2H), 5.52 (bs, 1H), 7.07 (d, J=8.6 Hz, 2H), 7.39 (t, J=8.1 Hz, 1H), 7.52 (d, J=8.0 Hz, 2H), 7.62 (d, J=8.5 Hz, 2H), 8.09 (s, 2H), 9.03 (s, 1H), 9.59 (bs, 1H). MS (ES+): m/z 456 (M+H)⁺.

### Example 144

### Testing of Kinase Inhibition

The ability of compounds of the present invention to inhibit the activity of three groups of kinases was tested. Kinases tested included the src family (primarily src and yes), the angiogenic growth factors receptors (FGFR1, PDGFRb and VEGFR2) and the ephrin, EphB4. All kinase reactions were conducted in 96-well plates with a final reaction volume of 50 ul.

Src Family

Recombinant human c-Src or Yes (28 ng/well, Panvera/Invitrogen, Madison Wis.), ATP (3 μM), a tyrosine kinase substrate (PTK2, 250 μM, Promega Corp., Madison Wis.), and test agents (at concentrations ranging from about 1 nM/l to about 100 μM/1), in the presence of Src kinase reaction buffer (Upstate USA, Lake Placid N.Y.). After reacting for about 90 minutes at room temperature, residual ATP was determined using a luciferase-based assay (KinaseGlo, Promega Corp.) as a measure of kinase activity. Data from four wells were then averaged and used to determine IC₅₀ values for the test compounds (Prism software package, GraphPad Software, San Diego Calif.).

Growth Factor Receptors

PDGFRb (0.16 ug/well, Panvera/Invitrogen) 500 nM ATP and the PTK2 peptide (700 uM) were combined with compound and reaction buffer as noted above for src. The reaction was incubated for 60 minutes at 37 C, and the residual ATP concentration was determined using the luciferase-based technique also noted above.

FGFR1 and VEGFR2 kinase assays were similarly performed. FGFR1 (76 ng/well, Panvera/Invitrogen) was combined with 12.5 mg/ml poly(glu4tyr) (Sigma) and 2.5 uM ATP. VEGFR2 (14.1 U/well, Cell Signaling/ProQinase) was used with 0.3 mg/ml poly(glu4tyr) and 1.5 uM ATP. Both were incubated for 60 minutes at 37 C, and the residual ATP was measured via luminescence, per the procedure described above.

EphB4

EphB4 kinase activity was similarly measured, using the luciferase-based technique described above. 28.9 mU/well EphB4 (Upstate) was reacted with 1 mg/ml poly(glu4tyr), 6 uM ATP and test reagents. The reaction was incubated for 60 minutes at 37 C and the residual ATP concentration was measured.

The test results for inhibition of Src kinase are presented in Table 1, and the test results for inhibition of some other kinases (i.e., Yes, Vegfr, EphB4, Pdgfrβ, and Fgfr1) are presented in Table 2. The abbreviation “IC₅₀” means that a particular compound of the invention, when present at the specified concentration, inhibited the kinase by 50%.

TABLE 1
Tests Results of Inhibition of Src Kinase by Some Compounds of the Invention

| | | Src IC₅₀ |
|---|---|---|
| Structure | Name | (nM) |
| 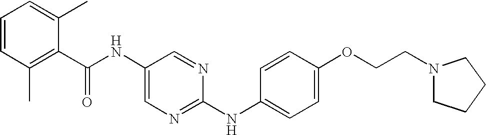 | 2,6-dimethyl-N-{2-[4-(2- pyrrolidin-1-yl-ethoxy)- phenylamino]-pyrimidin-5- yl}-benzamide | 104 |
| 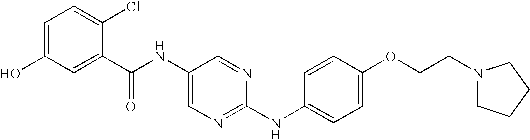 | 2-chloro-5-hydroxy-N-{2- [4-(2-pyrrolidin-1-yl- ethoxy)-phenylamino]- pyrimidin-5-yl}-benzamide | 27 |
| 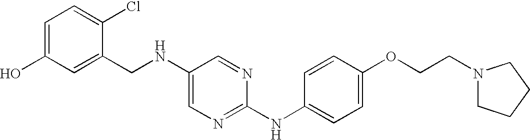 | 4-chloro-3-({2-[4-(2- pyrrolidin-1-yl-ethoxy)- phenylamino[-pyrimidin-5- ylamino}-methyl)-phenol | 55 |
| 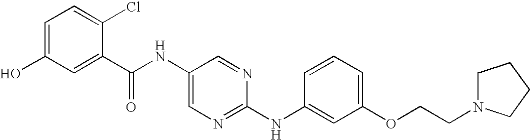 | 2-chloro-5-hydroxy-N-{2- [3-(2-pyrrolidin-1-yl- ethoxy)-phenylamino[- pyrimidin-5-yl}-benzamide | 21 |
| 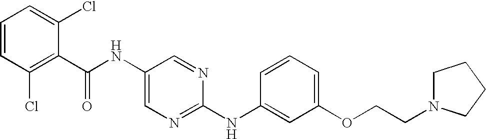 | 2,6-dichloro-N-{2-[3-(2- pyrrolidin-1-yl-ethoxy)- phenylamino[-pyrimidin-5- yl}-benzamide | 25 |
| 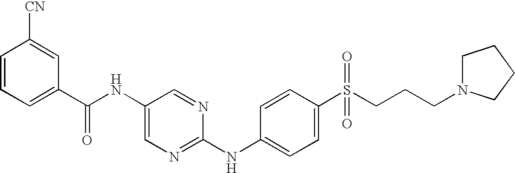 | 3-cyano-N-{2-[4-(3- pyrrolidin-1-yl-propane-1- sulfonyl)-phenylamino[- pyrimidin-5-yl}-benzamide | >10,000 |
| 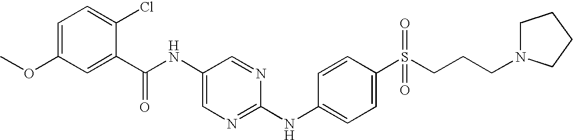 | 2-chloro-5-methoxy-N-{2- [4-(3-pyrrolidin-1-yl- propane-1-sulfonyl)- phenylamino[-pyrimidin-5- yl}-benzamide | 8000 |
| 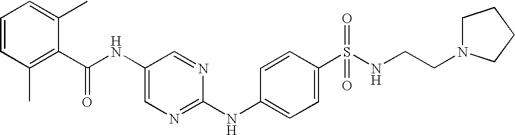 | 2,6-dimethyl-N-{2-[4-(2- pyrrolidin-1-yl- ethylsulfamoyl)- phenylamino]-pyrimidin-5- yl}-benzamide | 90 |
| 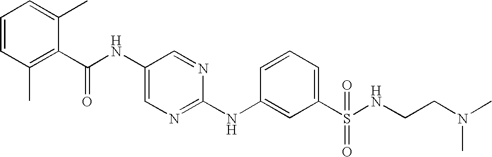 | N-{2-[3-(2-dimethylamino- ethylsulfamoyl)- phenylamino]-pyrimidin-5- yl}-2,6-dimethyl- benzamide | 288 |
| 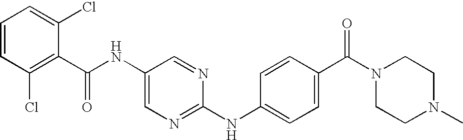 | 2,6-dichloro-N-{2-[4-(4- methyl-piperazine-1- carbonyl)-phenylamino]- pyrimidin-5-yl}-benzamide | 94 |
| 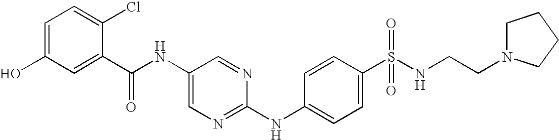 | 2-chloro-5-hydroxy-N-{2- [4-(2-pyrrolidin-1-yl- ethylsulfamoyl)- phenylamino]-pyrimidin-5- yl}-benzamide | 13 |
| 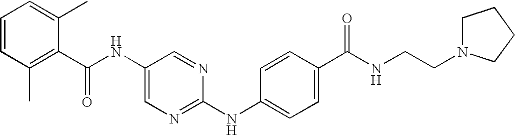 | 2,6-dimethyl-N-{2-[4-(2- pyrrolidin-1-yl- ethylcarbamoyl)- phenylamino]-pyrimidin-5- yl}-benzamide; compound with trifluoro-acetic acid | 117 |
| 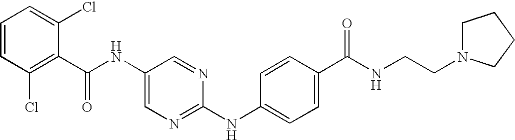 | 2,6-dichloro-N-{2-[4-(2- pyrrolidin-1-yl- ethylcarbamoyl)- phenylamino]-pyrimidin-5- yl}-benzamide; compound with trifluoro-acetic acid | 60 |
| 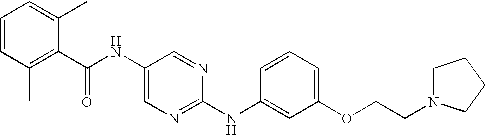 | 2,6-dimethyl-N-{2-[3-(2- pyrrolidin-1-yl-ethoxy)- phenylamino[-pyrimidin-5- yl}-benzamide | 99 |
| 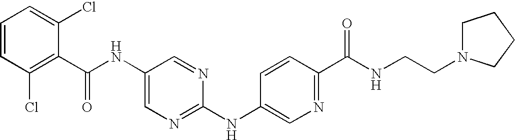 | 5-[5-(2,6-dichloro- benzoylamino)-pyrimidin- 2-ylamino[-pyridine-2- carboxylic acid (2- pyrrolidin-1-yl-ethyl)- amide | 129 |
| 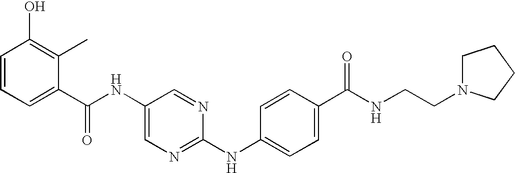 | 3-hydroxy-2-methyl-N-{2- [4-(2-pyrrolidin-1-yl- ethylcarbamoyl)- phenylamino[-pyrimidin-5- yl}-benzamide; compound with trifluoro-acetic acid | 216 |
| 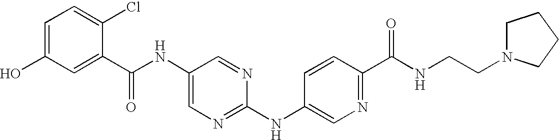 | 5-[5-(2-chloro-5-hydroxy- benzoylamino)-pyrimidin- 2-ylamino]-pyridine-2- carboxylic acid (2- pyrrolidin-1-yl-ethyl)- amide | 13 |
| 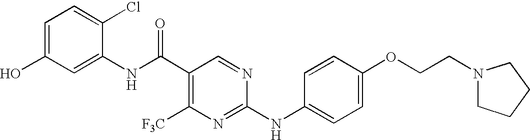 | 2-[4-(2-pyrrolidin-1-yl- ethoxy)-phenylamino[-4- trifluoromethyl- pyrimidine-5-carboxylic acid (2-chloro-5-hydroxy- phenyl)-amide | 1339 |
| 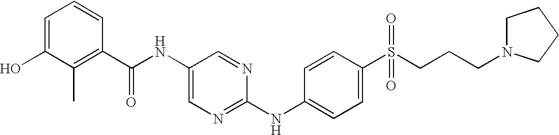 | 3-Hydroxy-2-methyl-N-{2- [4-(3-pyrrolidin-1-yl- propane-1-sulfonyl)- phenylamino]-pyrimidin-5- yl}-benzamide | 338 |
| 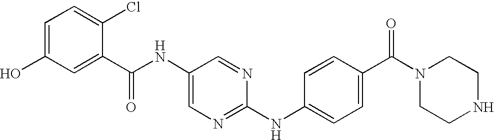 | 2-chloro-5-hydroxy-N-{2- [4-(piperazine-1-carbonyl)- phenylamino[-pyrimidin-5- yl}-benzamide hydrochloride | 12 |
| 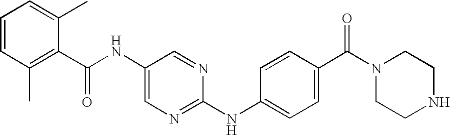 | 2,6-dimethyl-N-{2-[4- (piperazine-1-carbonyl)- phenylamino[-pyrimidin-5- yl}-benzamide | 183 |
| 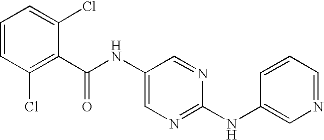 | 2,6-dichloro-N-[2-(pyridin- 3-ylamino)-pyrimidin-5- yl[-benzamide | 300 |
| 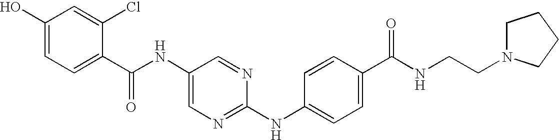 | 2-chloro-4-hydroxy-N-{2- [4-(2-pyrrolidin-1-yl- ethylcarbamoyl)- phenylamino]-pyrimidin-5- yl}-benzamide; compound with trifluoro-acetic acid | 85 |
| 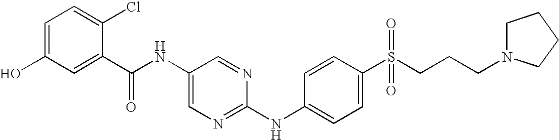 | 2-chloro-5-hydroxy-N-{2- [4-(3-pyrrolidin-1-yl- propane-1-sulfonyl)- phenylamino]-pyrimidin-5- yl}-benzamide | 9.8 |
| 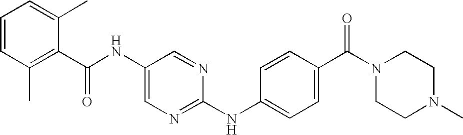 | 2,6-dimethyl-N-{2-[4-(4- methyl-piperazine-1- carbonyl)-phenylamino]- pyrimidin-5-yl}-benzamide | 223 |
| 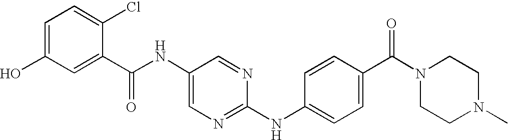 | 2-chloro-5-hydroxy-N-{2- [4-(4-methyl-piperazine-1- carbonyl)-phenylamino[- pyrimidin-5-yl}-benzamide | 21 |
| 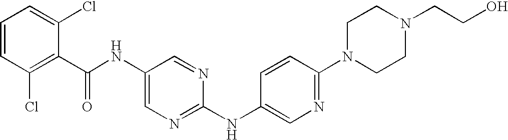 | 2,6-dichloro-N-(2-{6-[4- (2-hydroxy-ethyl)- piperazin- 1 -yl]-pyridin-3- ylamino}-pyrimidin-5-yl)- benzamide | 54 |
| 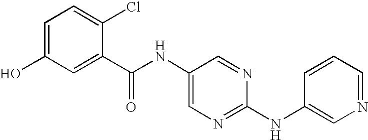 | 2-chloro-5-hydroxy-N-[2- (pyridin-3-ylamino)- pyrimidin-5-yl[-benzamide | 43 |
| 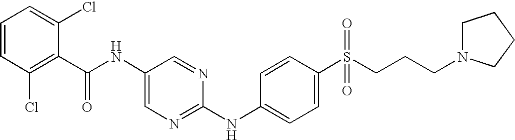 | 2,6-dichloro-N-{2-[4-(3- pyrrolidin-1-yl-propane-1- sulfonyl)-phenylamino]- pyrimidin-5-yl}-benzamide | 119 |
| 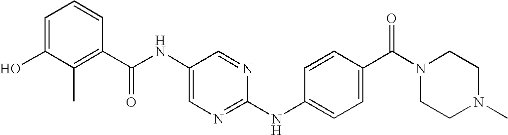 | 2-methyl-3-hydroxy-N-{2- [4-(4-methyl-piperazine-1- carbonyl)-phenylamimo]- pyrimidin-5-yl}-benzamide | 550 |
| 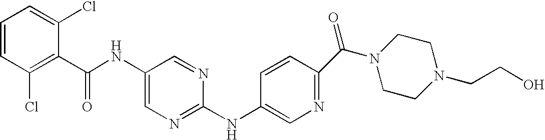 | 2,6-dichloro-N-(2-{6-[4- (2-hydroxy-ethyl)- piperazine-1-carbonyl]- pyridin-3-ylamino}- pyrimidin-5-yl)-benzamide | 400 |
| 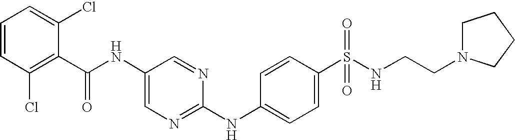 | 2,6-dichloro-N-{2-[4-(2- pyrrolidin-1-yl- ethylsulfamoyl)- phenylamino]-pyrimidin-5- yl}-benzamide | 73 |
| 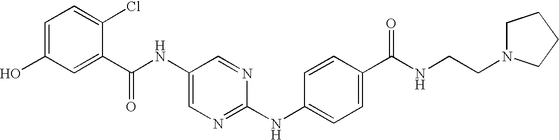 | 2-chloro-5-hydroxy-N-{2- [4-(2-pyrrolidin-1-yl- ethylcarbamoyl)- phenylamino]-pyrimidin-5- yl}-benzamide | 8.5 |
| 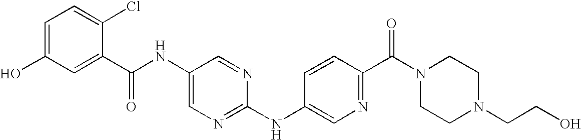 | 2-chloro-5-hydroxy-N-(2- {6-[4-(2-hydroxy-ethyl)- piperazine-1-carbonyl]- pyridin-3-ylamino}- pyrimidin-5-yl)-benzamide | 53 |
| 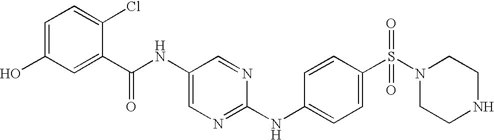 | 2-chloro-5-hydroxy-N-{2- [4-(piperazine-1-sulfonyl)- phenylamino]-pyrimidin-5- yl}-benzamide | 38 |
| 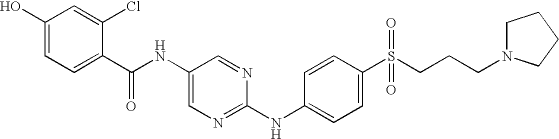 | 2-chloro-4-hydroxy-N-{2- [4-(3-pyrrolidin-1-yl- propane-1-sulfonyl)- phenylamino]-pyrimidin-5- yl}-benzamide | 462 |
| 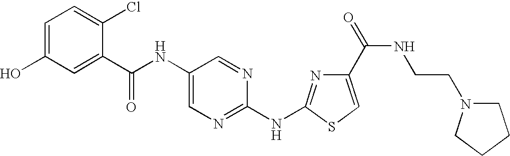 | 2-[5-(2-chloro-5-hydroxy- benzoylamino)-pyrimidin- 2-ylamino]-thiazole-4- carboxylic acid (2- pyrrolidin-1-yl-ethyl)- amide | 20 |
| 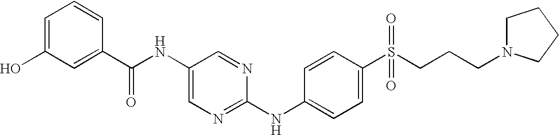 | 3-hydroxy-N-{2-[4-(3- pyrrolidin-1-yl-propane-1- sulfonyl)-phenylamino]- pyrimidin-5-yl}-benzamide | 1488 |
| 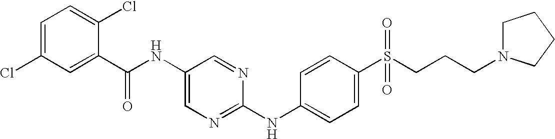 | 2,5-dichloro-N-{2-[4-(3- pyrrolidin-1-yl-propane-1- sulfonyl)-phenylamino]- pyrimidin-5-yl}-benzamide | 1748 |
| 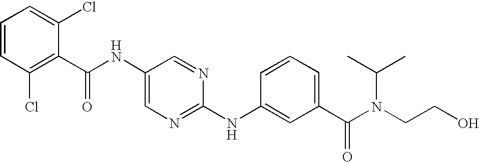 | 2,6-dichloro-N-(2-{3-[(2- hydroxy-ethyl)-isopropyl- carbamoyl[-phenylamino}- pyrimidin-5-yl)-benzamide | 25 |
| 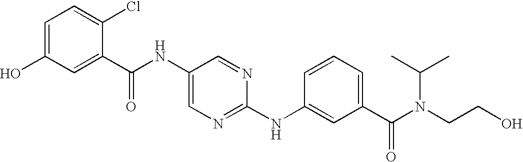 | 2-chloro-5-hydroxy-N-(2- {3-[(2-hydroxy-ethyl)- isopropyl-carbamoyl]- phenylamino}-pyrimidin- 5-yl)-benzamide | 19 |
| 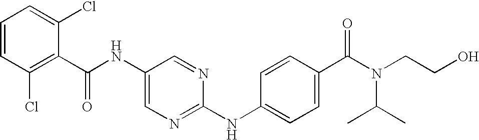 | 2,6-dichloro-N-(2-{4-[(2- hydroxy-ethyl)-isopropyl- carbamoyl]-phenylamino}- pyrimidin-5-yl)-benzamide | 286 |
| 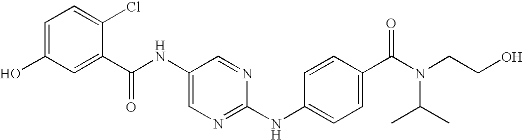 | 2-chloro-5-hydroxy-N-(2- 55 4-[(2-hydroxy-ethyl)- isopropyl-carbamoyl]- phenylamino}-pyrimidin- 5-yl)-benzamide | 43 |
| 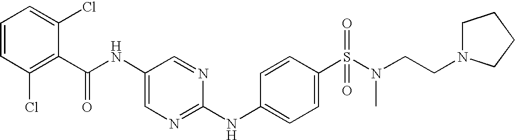 | 2,6-dichloro-N-(2-{4- [methyl-(2-pyrrolidin-1-yl- ethyl)-sulfamoyl]- phenylamino}-pyrimidin- 5-yl)-benzamide | 2100 |
| 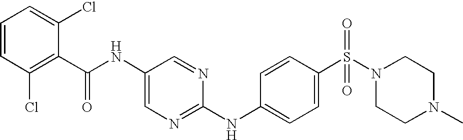 | 2,6-dichloro-N-{2-[4-(4- methyl-piperazine-1- sulfonyl)-phenylamino]- pyrimidin-5-yl}-benzamide | 667 |
| 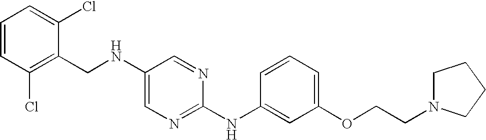 | N′-(2,6-dichloro-benzyl)- N-[3-(2-pyrrolidin-1-yl- ethoxy)-phenyl]- pyrimidine-2,5-diamine | 60 |
| 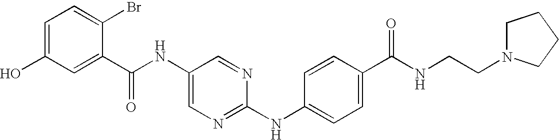 | 2-bromo-5-hydroxy-N-{2- [4-(2-pyrrolidin-1-yl- ethylcarbamoyl)- phenylamino]-pyrimidin-5- yl}-benzamide | 13 |
| 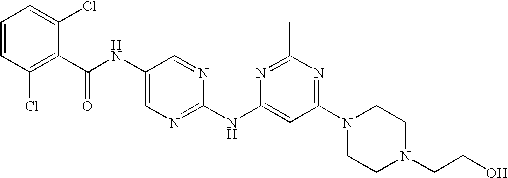 | 2,6-dichloro-N-(2-{6-[4- (2-hydroxy-ethyl)- piperazin-1-yl]-2-methyl- pyrimidin-4-ylamino}- pyrimidin-5-yl)-benzamide | 3000 |

TABLE 2
Tests Results of Inhibition of Selected Kinases by Some Compounds of the Invention
All data represent IC₅₀ in nM.

| Structure | Yes | Vegfr | EphB4 | Pdgfrβ | Fgfr1 |
|---|---|---|---|---|---|
| 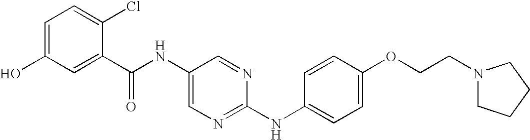 | 26 | 564 | 200 | 20 | 20000 |
| 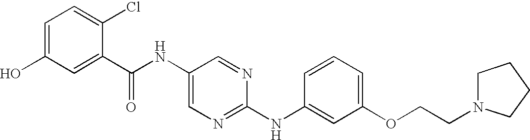 | 10 | 1264 | 109 | 13 | 7200 |
| 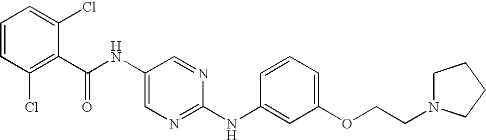 | 8 | >10,000 | 434 | 181 | >10000 |
| 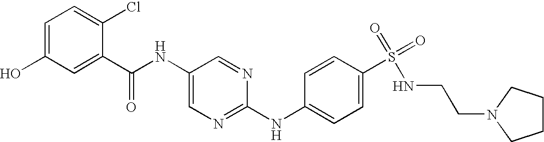 | 0.8 | 322 | | | 5200 |
| 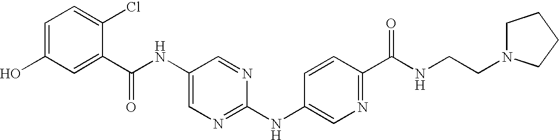 | 3.1 | 870 | 89 | 13 | 1000 |
| 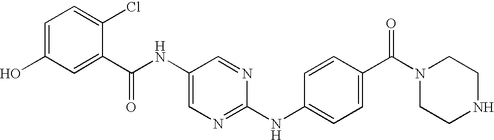 | 1.9 | 310 | | | 5400 |
| 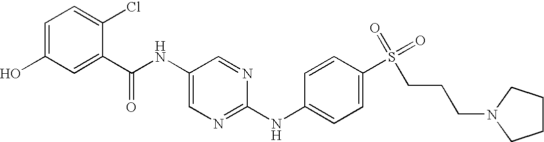 | 3.7 | 358 | | | 7500 |
| 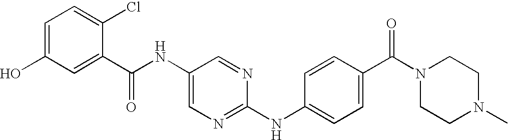 | 10 | 360 | | | 9400 |
| 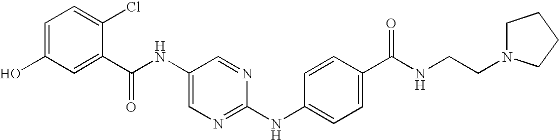 | 4.4 | 257 | 59 | 6.3 | 8200 |
| 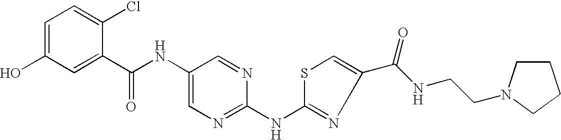 | 19 | 614 | | | 2100 |
| 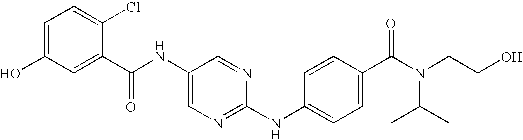 | 6.0 | >10,000 | 190 | 138 | >10000 |
| 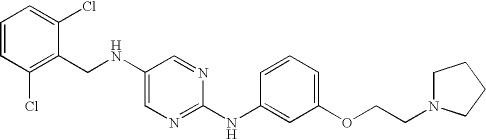 | 6.0 | 138 | 190 | | >10,000 |
| 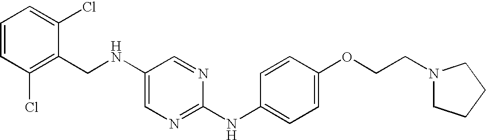 | 40 | 907 | | 51 | 5278 |
| 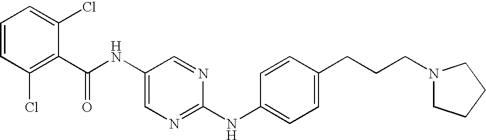 | 26 | >10,000 | | 206 | >10,000 |

Although the invention has been described with reference to the above examples, it will be understood that modifications and variations are encompassed within the spirit and scope of the invention. Accordingly, the invention is limited only by the following claims.

What is claimed is:

1. A compound of structure A: 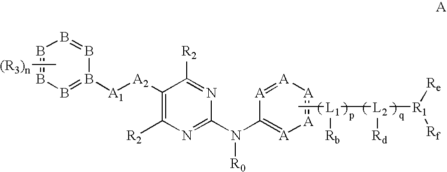
wherein
each of A is independently selected from a group consisting of CH, N, NH, O, and S, or A is a part of a ring fusion to form a second ring, wherein the second ring is selected from a group consisting of an aromatic, a heteroaromatic, a bicyclic aromatic, and a bicyclic aromatic heterocyclic ring;
each of B is, independently, CH, or a part of a ring fusion to form a second ring, wherein the second ring selected from a group consisting of an aromatic, a bicyclic aromatic, and a bicyclic, with the further proviso that when the second ring is present, only the first ring is aromatic;
A₁ is selected from a group consisting of NRₐ, C(O), S(O), S(O)₂, P(O)₂, O, S, and CRₐ, wherein R is selected from a group consisting of H, lower alkyl, branched alkyl, hydroxyalkyl, aminoalkyl, thioalkyl, alkylhydroxyl, alklythiol, and alkylamino, and wherein if A₁ is Nₐ, a=1, and if A₁ is CRₐ, a=2;
A₂ is selected from a group consisting of NR, C(O), S(O), S(O)₂, P(O)₂, O, and S; and the connectivity between A₁ and A₂ is chemically correct;
R₀ is selected from a group consisting of H, a lower alkyl, and a branched alkyl;
L₁ is selected from a group consisting of a bond, O, S, C(O), S(O), S(O)₂, NRₐ, and C₁-C₆ alkyl; L₂ is selected from a group consisting of a bond, O, S, C(O), S(O), S(O)₂, C₁-C₆, and NRₐ; or L₁ and L₂ taken together is a bond;
each of R_{b}, R_{d}, Rₑ, R_{f} either is absent or is selected from a group consisting of H, C₁-C₆ alkyl, cycloalkyl, branched alkyl, hydroxy alkyl, aminoalkyl, thioalkyl, alkylhydroxyl, alkklythiol, and alkylamino;
each of p, q, m, r is independently an integer having value from 0 to 6;
R_{b} and R_{d} taken together is a moiety selected from a group consisting of (CH₂)ₘ, (CH₂)ᵣ—S—(CH₂)ₘ, (CH₂)ᵣ—SO—(CH₂)ₘ, CH₂)ᵣ—SO₂—(CH₂)ₘ, (CH₂)ᵣ—NRₐ—(CH₂)ₘ, and (CH₂)ᵣ—O—(CH₂)ₘ; or
R_{b} and Rₑ taken together is a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ; or
R_{d} and R_{f} taken together is a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ; or
R_{b} and R_{f} taken together is a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ; or
R_{d} and Rₑ taken together is a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣNRₐ—(CH₂)ₘ, and (CH₂)ᵣ—O—(CH₂)ₘ;
R₁ is selected from a group consisting of (CRₐ)ₘ, O, N, S, C(O)(O)R′, C(O)N(R′)₂, SO₃R′, OSO₂R′, SO₂R′, SOR′, PO₄R′, OPO₂R′, PO₃R′, PO₂R′, and a 3-6 membered heterocycle with one or more heterocyclic atoms, wherein R′ is selected from a group consisting of hydrogen, lower alkyl, alkyl-hydroxyl, or forms a closed 3-6 membered heterocycle with one or more heterocyclic atoms, branched alkyl, branched alkyl hydroxyl, wherein each R′ is independent in case there is more than one R′;
R₂ is selected from a group consisting of hydrogen, alkyl, branched alkyl, phenyl, substituted phenyl, halogen, alkylamino, alkyloxo, CF₃, sulfonamido, substituted sulfonamido, alkyoxy, thioalkyl, sulfonate, sulfonate ester, phosphate, phosphate ester, phosphonate, phosphonate ester, carboxo, amido, ureido, substituted carboxo, substituted amido, substituted ureido, and 3-6 membered heterocycle with one or more hetrocyclic atoms, with the further proviso that either one or two substituents R₂ can be present in the ring, and if more than one substituent R₂ are present, each of the substituents R₂ can be the same or different;
R₃ is selected from a group consisting of hydrogen, alkyl, branched alkyl, alkoxy, halogen, CF₃, cyano, substituted alkyl, hydroxyl, alklylhydroxyl, thiol, alkylthiol, thioalkyl, amino, and aminoalkyl; and
n is an integer having value between 1 and 5, with the further proviso that if n≧2, then each group R₃ is independent of the other groups R₃.

2. The compound of claim 1, wherein the compound is selected from the group consisting of compounds XXXVIII and XXXIX:

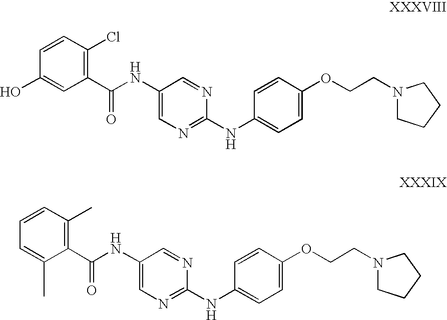

3. The compound of claim 1, wherein the compound is is selected from the group consisting of compound XII and XLI:

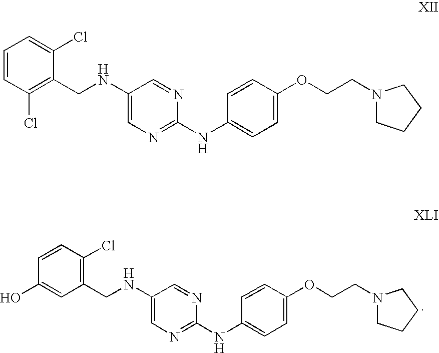

4. The compound of claim 1, wherein the compound is selected from the group consisting of compounds XLII, XLIII and XLIV:

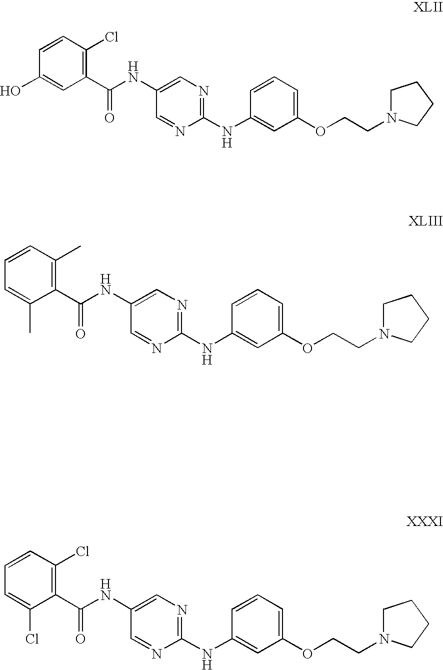

5. The compound of claim 1, wherein the compound is selected from the group consisting of compounds XXXI, XXXII, XXXIII, XXXIV, XXXV, XXXVI, XXXVII, LIV, LV, and LX:

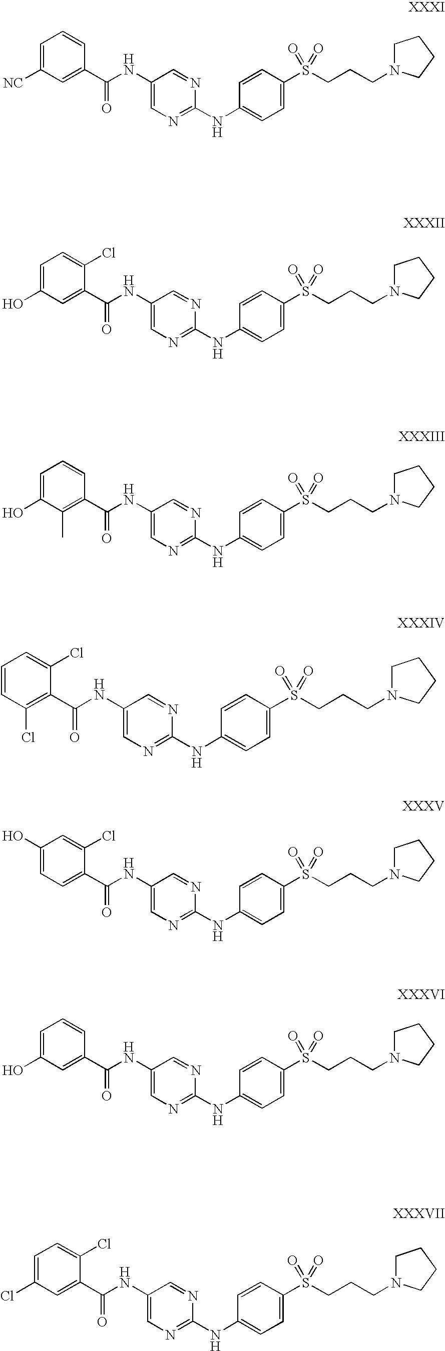

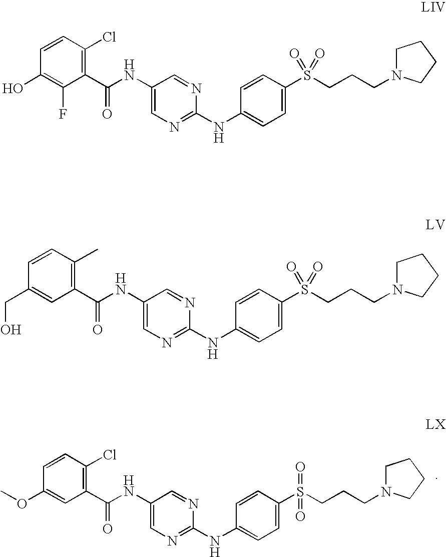

6. The compound of claim 1, wherein the compound is selected from the group consisting of compounds III, IV, VI, and VII:

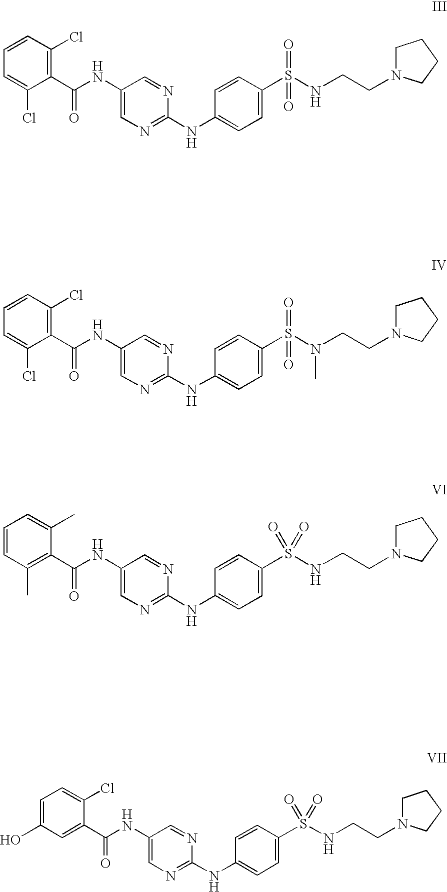

7. The compound of claim 1, wherein the compound is selected from the group consisting of compounds I and LXI:

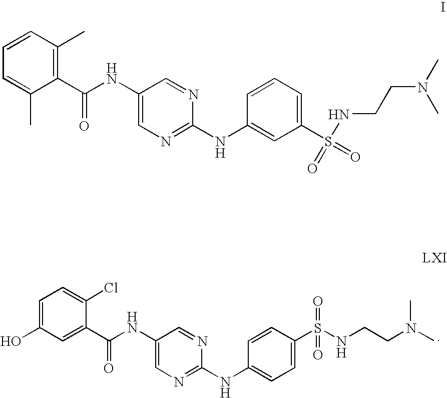

8. The compound of claim 1, wherein the compound is selected from the group consisting of compounds XIV, XV, XVI, XVII, L, and LI:

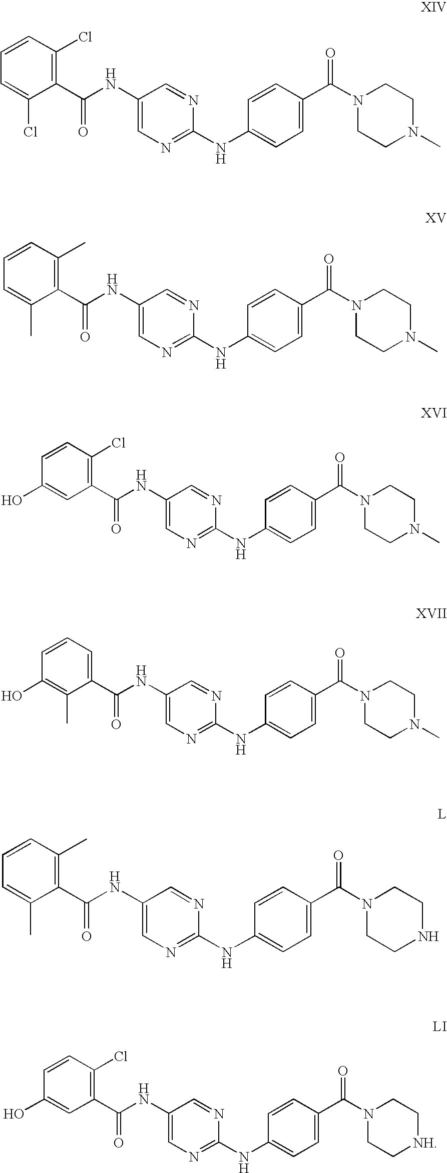

9. The compound of claim 1, wherein the compound is selected from the group consisting of compounds XXIII, XXV, XXVI, XXVII, XXVIII, and XXIX:

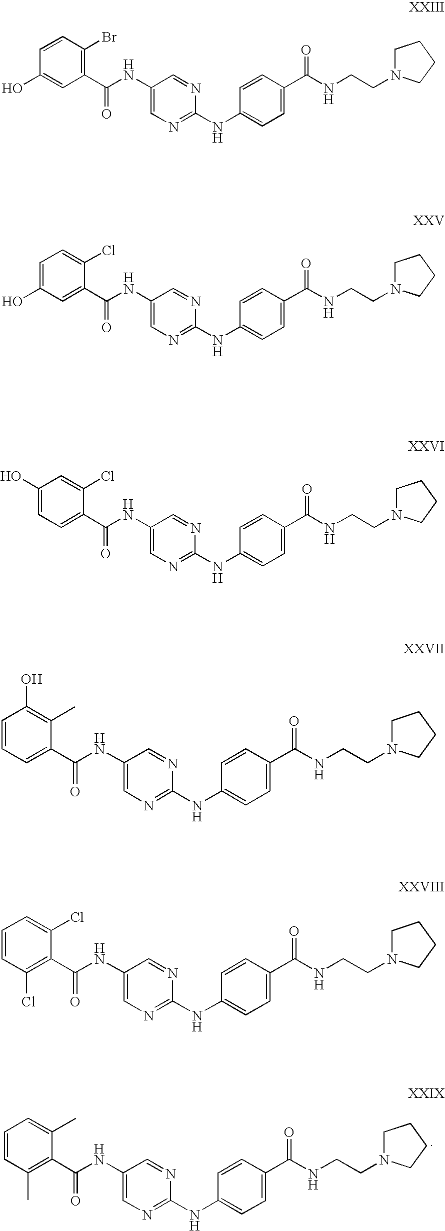

10. The compound of claim 1, wherein the compound is selected from the group consisting of compounds VIII and IX:

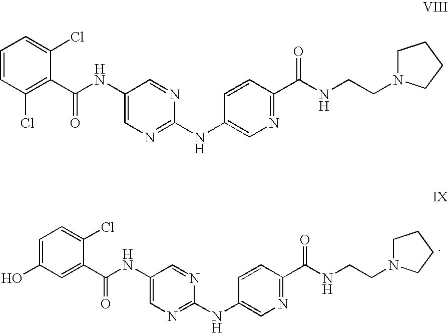

11. The compound of claim 1, wherein the compound is selected from the group consisting of compounds II and XLVIII:

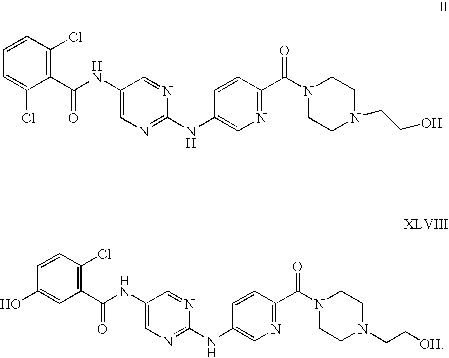

12. The compound of claim 1, wherein the compound is selected from the group consisting of compounds V and XLVII:

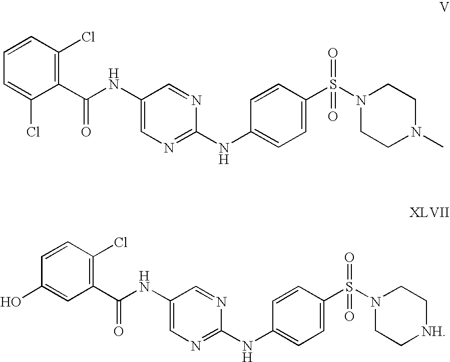

13. The compound of claim 1, wherein the compound is compound LXVII:

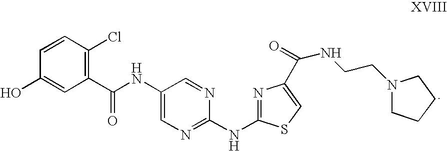

14. The compound of claim 1, wherein the compound is compound LXII:

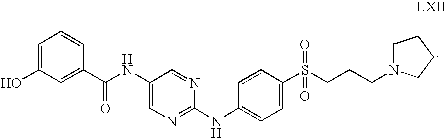

15. The compound of claim 1, wherein the compound is selected from the group consisting of compounds XXI and XXII:

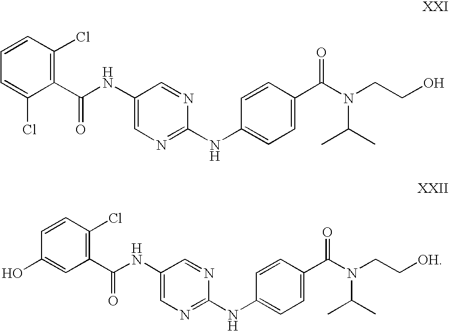

16. The compound of claim 1, wherein the compound is selected from the group consisting of compounds XIX and XX:

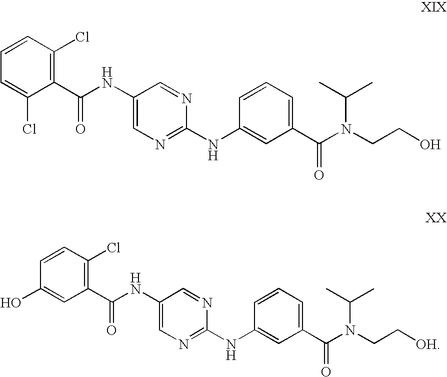

17. The compound of claim 1, wherein the compound is compound XXX:

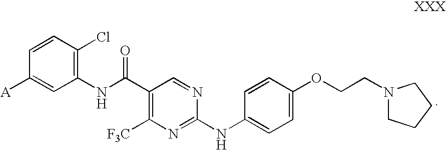

18. The compound of claim 1, wherein the compound is compound LII:

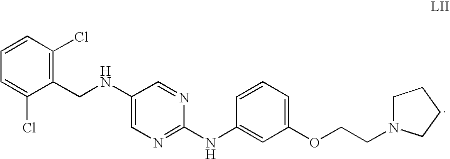

19. The compound of claim 1, wherein the compound is compound XLIX:

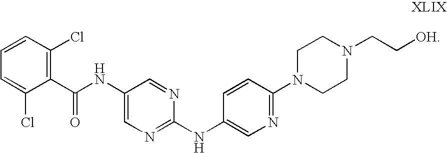

20. The compound of claim 1, wherein the compound is selected from the group consisting of compounds X and XI:

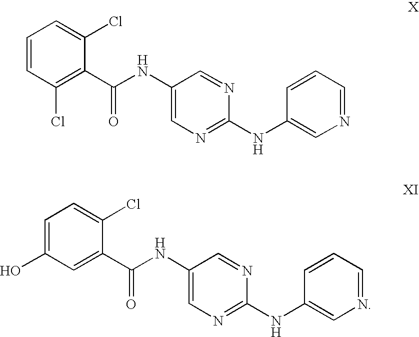

21. The compound of claim 1, wherein the compound is compound XIII:

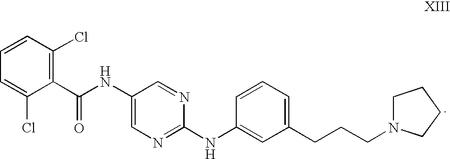

22. The compound of claim 1, wherein the compound is compound XLVI:

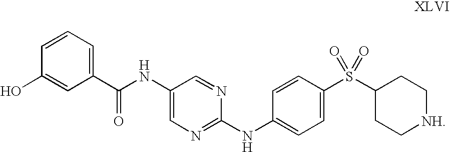

23. The compound of claim 1, wherein the compound is compound LIII:

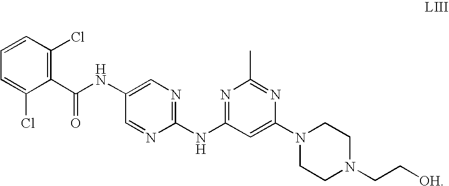

24. The compound of claim 1, wherein the compound is selected from the group consisting of compounds LVI and LVII:

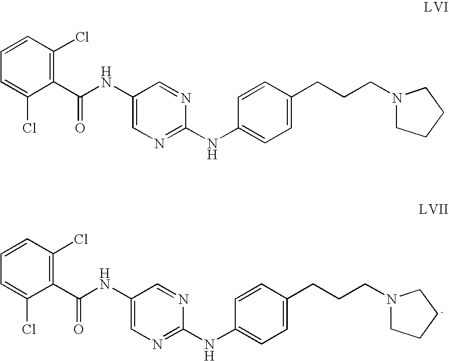

25. A method for treating a disorder associated with compromised vasculostasis, comprising administering to a subject in need thereof a therapeutically effective amount of at least one compound of claim 1.

26. The method of claim 25, wherein the disorder is myocardial infarction, stroke, congestive heart failure, an ischemia or reperfusion injury, cancer, arthritis or other arthropathy, retinopathy or vitreoretinal disease, macular degeneration, autoimmune disease, vascular leakage syndrome, inflammatory disease, edema, transplant rejection, burn, or acute or adult respiratory distress syndrome (ARDS).

27. The method of claim 25, wherein the disorder is vascular leakage syndrome (VLS).

28. The method of claim 25, wherein the disorder is cancer.

29. The method of claim 25, wherein the disorder is an ophthalmological disease.

30. The method of claim 29, wherein the ophthalmological disease is selected from a group consisting of age-related macular degeneration (AMD), diabetic retinopathy (DR), diabetic macular edema (DME), cancer, glaucoma, and other pathological conditions of the eye.

31. The method of claim 29, wherein the compound is administered to the back of the eye, intravitreally, or periocularly.

32. The method of claim 29, wherein the compound is in a formulation in the form of eye-drops.

33. The method of claim 29, wherein the compound is administered to a subject having dry AMD.

34. The method of claim 29, wherein the compound is adminstered to reduce the risk of progression of the ophthalmological disease.

35. The method of claim 25, wherein the disorder is ARDS.

36. The method of claim 25, wherein the disorder is autoimmune disease.

37. The method of claim 25, wherein the disorder is burn.

38. The method of claim 25, wherein the disorder is stroke.

39. The method of claim 25, wherein the disorder is myocardial infarction.

40. The method of claim 25, wherein the disorder is ischemia or reperfusion injury.

41. The method of claim 26, wherein the disorder is arthritis.

42. The method of claim 25, wherein the disorder is edema.

43. The method of claim 25, wherein the disorder is transplant rejection.

44. The method of claim 25, wherein the disorder is inflammatory disease.

45. The method of claim 25, wherein the disorder is congestive heart failure.

46. The method of claim 25, wherein the disorder is associated with a kinase.

47. The method of claim 46, wherein the kinase is a tyrosine kinase.

48. The method of claim 46, wherein the kinase is a serine kinase or a threonine kinase.

49. The method of claim 46, wherein the kinase is a Src family kinase.

50. A pharmaceutical composition comprising at least one compound of claim 1 and a pharmaceutically acceptable carrier therefore.

51. An article of manufacture comprising packaging material and a pharmaceutical composition contained within the packaging material, wherein the packaging material comprises a label which indicates that the pharmaceutical composition can be used for treatment of disorders associated with compromised vasculostasis, and wherein the pharmaceutical composition comprises at least one compound of claim 1.

52. An article of manufacture comprising packaging material and a pharmaceutical composition contained within the packaging material, wherein the packaging material comprises a label which indicates that the pharmaceutical composition can be used for treatment of disorders associated with vascular permeability leakage or compromised vasculostasis selected from myocardial infarction, stroke, congestive heart failure, an ischemia or reperfusion injury, cancer, arthritis or other arthropathy, retinopathy or another ophthalmological disease, macular degeneration, autoimmune disease, vascular leakage syndrome, inflammatory disease, edema, transplant rejection, burn, or acute or adult respiratory distress syndrome (ARDS), and wherein the pharmaceutical composition comprises at least one compound of claim 1.

53. The article of manufacture of claim 52, wherein the disorder is cancer.

54. A method of treating a disorder associated with compromised vasculostasis, comprising the administration of a therapeutically effective amount of at least one compound of claim 1 or pharmaceutically acceptable salts, hydrates, solvates, crystal forms and individual diastereomers thereof, to a subject in need of such treatment.

55. The method of claim 54, wherein the disorder is vascular leakage syndrome (VLS).

56. The method of claim 54, wherein the disorder is cancer.

57. The method of claim 54, wherein the disorder is an ophthalmological disease.

58. The method of claim 54, wherein the disorder is ARDS.

59. The method of claim 54, wherein the disorder is an autoimmune disease.

60. The method of claim 54, wherein the disorder is burn.

61. The method of claim 54, wherein the disorder is stroke.

62. The method of claim 54, wherein the disorder is myocardial infarction.

63. The method of claim 54, wherein the disorder is ischemia or reperfusion injury.

64. The method of claim 54, wherein the disorder is arthritis.

65. The method of claim 54, wherein the disorder is edema.

66. The method of claim 54, wherein the disorder is transplant rejection.

67. The method of claim 54, wherein the disorder is inflammatory disease.

68. A method of treating a disorder associated with compromised vasculostasis comprising the administration of a therapeutically effective amount of at least one compound of claim 1, or pharmaceutically acceptable salts, hydrates, solvates, crystal forms and individual diastereomers thereof, in combination with an anti-inflammatory agent, chemotherapeutic agent, immunomodulatory agent, therapeutic antibody, or a protein kinase inhibitor, to a subject in need of such treatment.

69. A method of treating a subject having or at risk of having myocardial infarction comprising administering to the subject a therapeutically effective amount of at least one compound of claim 1, thereby treating the subject.

70. A method of treating a subject having or at risk of having vascular leakage syndrome (VLS) comprising administering to the subject a therapeutically effective amount of at least one compound of claim 1, thereby treating the subject.

71. A method of treating a subject having or at risk of having cancer comprising administering to the subject a therapeutically effective amount of at least one compound of claim 1, thereby treating the subject.

72. A method of treating a subject having or at risk of having stroke comprising administering to the subject a therapeutically effective amount of at least one compound of claim 1, thereby treating the subject.

73. A method of treating a subject having or at risk of having ARDS comprising administering to the subject a therapeutically effective amount of at least one compound of claim 1, thereby treating the subject.

74. A method of treating a subject having or at risk of having burns comprising administering to the subject a therapeutically effective amount of at least one compound of claim 1, thereby treating the subject.

75. A method of treating a subject having or at risk of having arthritis comprising administering to the subject a therapeutically effective amount of at least one compound of claim 1, thereby treating the subject.

76. A method of treating a subject having or at risk of having edema comprising administering to the subject a therapeutically effective amount of at least one compound of claim 1, thereby treating the subject.

77. A method of treating a subject having or at risk of having vascular leakage syndrome (VLS) comprising administering to the subject a therapeutically effective amount of at least one compound of claim 1, thereby treating the subject.

78. A method of treating a subject having or at risk of having retinopathy or another ophthalmological disease comprising administering to the subject a therapeutically effective amount of at least one compound of claim 1, thereby treating the subject.

79. A method of treating a subject having or at risk of having ischemic or reperfusion related tissue injury or damage, comprising administering to the subject a therapeutically effective amount of at least one compound of claim 1, thereby treating the subject.

80. A method of treating a subject having or at risk of having an autoimmune disease, comprising administering to the subject a therapeutically effective amount of at least one compound of claim 1, thereby treating the subject.

81. A method of treating a subject having or at risk of having transplant rejection, comprising administering to the subject a therapeutically effective amount of at least one compound of claim 1, thereby treating the subject.

82. A method of treating a subject having or at risk of having inflammatory disease, comprising administering to the subject a therapeutically effective amount of at least one compound of claim 1, thereby treating the subject.

83. A process for making a pharmaceutical composition comprising combining a combination of at least one compound of claim 1 or its pharmaceutically acceptable salts, hydrates, solvates, crystal forms salts and individual diastereomers thereof, and a pharmaceutically acceptable carrier.
